SEQUENCE LISTING (122 sequences)

SEQ ID NO: 1 - protein, 1048 aa, Homo sapiens
MAFPPRRRLRLGPRGLPLLLSGLLLPLCRAFNLDVDSPAEYSGPEGSYFGFAVDFFVPSASSRMFLLVGAPKANTTQPGIVEGGQVLKCDWSSTRRCQPIEFDATGNRDYAKDDPLEFKSHQWFGASVRSKQDKILACAPLYHWRTEMKQEREPVGTCFLQDGTKTVEYAPCRSQDIDADGQGFCQGGFSIDFTKADRVLLGGPGSFYWQGQLISDQVAEIVSKYDPNVYSIKYNNQLATRTAQAIFDDSYLGYSVAVGDFNGDGIDDFVSGVPRAARTLGMVYIYDGKNMSSLYNFTGEQMAAYFGFSVAATDINGDDYADVFIGAPLFMDRGSDGKLQEVGQVSVSLQRASGDFQTTKLNGFEVFARFGSAIAPLGDLDQDGFNDIAIAAPYGGEDKKGIVYIFNGRSTGLNAVPSQILEGQWAARSMPPSFGYSMKGATDIDKNGYPDLIVGAFGVDRAILYRARPVITVNAGLEVYPSILNQDNKTCSLPGTALKVSCFNVRFCLKADGKGVLPRKLNFQVELLLDKLKQKGAIRRALFLYSRSPSHSKNMTISRGGLMQCEELIAYLRDESEFRDKLTPITIFMEYRLDYRTAADTTGLQPILNQFTPANISRQAHILLDCGEDNVCKPKLEVSVDSDQKKIYIGDDNPLTLIVKAQNQGEGAYEAELIVSIPLQADFIGVVRNNEALARLSCAFKTENQTRQVVCDLGNPMKAGTQLLAGLRFSVHQQSEMDTSVKFDLQIQSSNLFDKVSPVVSHKVDLAVLAAVEIRGVSSPDHVFLPIPNWEHKENPETEEDVGPVVQHIYELRNNGPSSFSKAMLHLQWPYKYNNNTLLYILHYDIDGPMNCTSDMEINPLRIKISSLQTTEKNDTVAGQGERDHLITKRDLALSEGDIHTLGCGVAQCLKIVCQVGRLDRGKSAILYVKSLLWTETFMNKENQNHSYSLKSSASFNVIEFPYKNLPIEDITNSTLVTTNVTWGIQPAPMPVPVWVIILAVLAGLLLLAVLVFVMYRMGFFKRVRPPQEEQEREQLQPHENGEGNSET

SEQ ID NO: 2 - protein, 798 aa, Homo sapiens
MNLQPIFWIGLISSVCCVFAQTDENRCLKANAKSCGECIQAGPNCGWCTNSTFLQEGMPTSARCDDLEALKKKGCPPDDIENPRGSKDIKKNKNVTNRSKGTAEKLKPEDITQIQPQQLVLRLRSGEPQTFTLKFKRAEDYPIDLYYLMDLSYSMKDDLENVKSLGTDLMNEMRRITSDFRIGFGSFVEKTVMPYISTTPAKLRNPCTSEQNCTSPFSYKNVLSLTNKGEVFNELVGKQRISGNLDSPEGGFDAIMQVAVCGSLIGWRNVTRLLVFSTDAGFHFAGDGKLGGIVLPNDGQCHLENNMYTMSHYYDYPSIAHLVQKLSENNIQTIFAVTEEFQPVYKELKNLIPKSAVGTLSANSSNVIQLIIDAYNSLSSEVILENGKLSEGVTISYKSYCKNGVNGTGENGRKCSNISIGDEVQFEISITSNKCPKKDSDSFKIRPLGFTEEVEVILQYICECECQSEGIPESPKCHEGNGTFECGACRCNEGRVGRHCECSTDEVNSEDMDAYCRKENSSEICSNNGECVCGQCVCRKRDNTNEIYSGKFCECDNFNCDRSNGLICGGNGVCKCRVCECNPNYTGSACDCSLDTSTCEASNGQICNGRGICECGVCKCTDPKFQGQTCEMCQTCLGVCAEHKECVQCRAFNKGEKKDTCTQECSYFNITKVESRDKLPQPVQPDPVSHCKEKDVDDCWFYFTYSVNGNNEVMVHVVENPECPTGPDIIPIVAGVVAGIVLIGLALLLIWKLLMIIHDRREFAKFEKEKMNAKWDTGENPIYKSAVTTVVNPKYEGK

SEQ ID NO: 3 - protein, 5 aa, Artificial Sequence
  source: /note=“Description of Artificial Sequence Synthetic peptide”
SYYMH

SEQ ID NO: 4 - protein, 9 aa, Artificial Sequence
  source: /note=“Description of Artificial Sequence Synthetic peptide”
YTFTSYYMH

SEQ ID NO: 5 - protein, 17 aa, Artificial Sequence
  source: /note=“Description of Artificial Sequence Synthetic peptide”
IINPSGGSTSYAQKFQG

SEQ ID NO: 6 - protein, 10 aa, Artificial Sequence
  source: /note=“Description of Artificial Sequence Synthetic peptide”
IINPSGGSTS

SEQ ID NO: 7 - protein, 16 aa, Artificial Sequence
  source: /note=“Description of Artificial Sequence Synthetic peptide”
QQRHRRDYDYYYGMDV

SEQ ID NO: 8 - protein, 12 aa, Artificial Sequence
  source: /note=“Description of Artificial Sequence Synthetic peptide”
RASQSVSSDYLA

SEQ ID NO: 9 - protein, 7 aa, Artificial Sequence
  source: /note=“Description of Artificial Sequence Synthetic peptide”
GASRRAT

SEQ ID NO: 10 - protein, 9 aa, Artificial Sequence
  source: /note=“Description of Artificial Sequence Synthetic peptide”
QQAYSLPPT

SEQ ID NO: 11 - protein, 125 aa, Artificial Sequence
  source: /note=“Description of Artificial Sequence Synthetic polypeptide”
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARQQRHRRDYDYYYGMDVWGQGTTVTVSS

SEQ ID NO: 12 - protein, 108 aa, Artificial Sequence
  source: /note=“Description of Artificial Sequence Synthetic polypeptide”
EIVLTQSPGTLSLSPGERATLSCRASQSVSSDYLAWYQQKPGQAPRLLIYGASRRATGIPDRFSGSGSGTDFTLTISRLEPEDFAVYYCQQAYSLPPTFGGGTKVEIK

SEQ ID NO: 13 - protein, 5 aa, Artificial Sequence
  source: /note=“Description of Artificial Sequence Synthetic peptide”
SYGMH

SEQ ID NO: 14 - protein, 9 aa, Artificial Sequence
  source: /note=“Description of Artificial Sequence Synthetic peptide”
FTFSSYGMH

SEQ ID NO: 15 - protein, 17 aa, Artificial Sequence
  source: /note=“Description of Artificial Sequence Synthetic peptide”
VISYDGSNKYYADSVKG

SEQ ID NO: 16 - protein, 10 aa, Artificial Sequence
  source: /note=“Description of Artificial Sequence Synthetic peptide”
VISYDGSNKY

SEQ ID NO: 17 - protein, 18 aa, Artificial Sequence
  source: /note=“Description of Artificial Sequence Synthetic peptide”
GGPTRGDGTRVYYYGMDV

SEQ ID NO: 18 - protein, 11 aa, Artificial Sequence
  source: /note=“Description of Artificial Sequence Synthetic peptide”
RASQSVSSNLA

SEQ ID NO: 19 - protein, 7 aa, Artificial Sequence
  source: /note=“Description of Artificial Sequence Synthetic peptide”
SASTRAT

SEQ ID NO: 20 - protein, 9 aa, Artificial Sequence
  source: /note=“Description of Artificial Sequence Synthetic peptide”
QQYYHHPFT

SEQ ID NO: 21 - protein, 127 aa, Artificial Sequence
  source: /note=“Description of Artificial Sequence Synthetic polypeptide”
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCARGGPTRGDGTRVYYYGMDVWGQGTTVTVSS

SEQ ID NO: 22 - protein, 107 aa, Artificial Sequence
  source: /note=“Description of Artificial Sequence Synthetic polypeptide”
EIVMTQSPATLSVSPGERATLSCRASQSVSSNLAWYQQKPGQAPRLLIYSASTRATGIPARFSGSGSGTEFTLTISSLQSEDFAVYYCQQYYHHPFTFGGGTKVEIK

SEQ ID NO: 23 - protein, 13 aa, Artificial Sequence
  source: /note=“Description of Artificial Sequence Synthetic peptide”
ETNYYRGGPAFDI

SEQ ID NO: 24 - protein, 16 aa, Artificial Sequence
  source: /note=“Description of Artificial Sequence Synthetic peptide”
RSSQSLLHSNGYNYLD

SEQ ID NO: 25 - protein, 7 aa, Artificial Sequence
  source: /note=“Description of Artificial Sequence Synthetic peptide”
LGSNRAS

SEQ ID NO: 26 - protein, 10 aa, Artificial Sequence
  source: /note=“Description of Artificial Sequence Synthetic peptide”
MQVLGTPPWT

SEQ ID NO: 27 - protein, 122 aa, Artificial Sequence
  source: /note=“Description of Artificial Sequence Synthetic polypeptide”
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARETNYYRGGPAFDIWGQGTMVTVSS

SEQ ID NO: 28 - protein, 113 aa, Artificial Sequence
  source: /note=“Description of Artificial Sequence Synthetic polypeptide”
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQVLGTPPWTFGGGTKVEIK

SEQ ID NO: 29 - protein, 9 aa, Artificial Sequence
  source: /note=“Description of Artificial Sequence Synthetic peptide”
FTFTSYYMH

SEQ ID NO: 30 - protein, 125 aa, Artificial Sequence
  source: /note=“Description of Artificial Sequence Synthetic polypeptide”
QVQLVESGGGLVQPGGSLRLSCAASGFTFTSYYMHWVRQAPGQGLEWMGIINPSGGSTSYAQKFQGRVTMTRDTSTSTVYMELSSLRSEDTAVYYCARQQRHRRDYDYYYGMDVWGQGTTVTVSS

SEQ ID NO: 31 - protein, 5 aa, Artificial Sequence
  source: /note=“Description of Artificial Sequence Synthetic peptide”
YYGMH

SEQ ID NO: 32 - protein, 9 aa, Artificial Sequence
  source: /note=“Description of Artificial Sequence Synthetic peptide”
FTFKYYGMH

SEQ ID NO: 33 - protein, 17 aa, Artificial Sequence
  source: /note=“Description of Artificial Sequence Synthetic peptide”
SIWYDGSNKKYADSVKG

SEQ ID NO: 34 - protein, 10 aa, Artificial Sequence
  source: /note=“Description of Artificial Sequence Synthetic peptide”
SIWYDGSNKK

SEQ ID NO: 35 - protein, 127 aa, Artificial Sequence
  source: /note=“Description of Artificial Sequence Synthetic polypeptide”
EVQLVESGGGVVQPGRSLRLSCAASGFTFKYYGMHWVRQAPGKGLEWVASIWYDGSNKKYADSVKGRFTISRDNSKNTLYLQMNSLRAEDTAVYYCARGGPTRGDGTRVYYYGMDVWGQGTTVTVSS

SEQ ID NO: 36 - protein, 5 aa, Artificial Sequence
  source: /note=“Description of Artificial Sequence Synthetic peptide”
DYYMS

SEQ ID NO: 37 - protein, 9 aa, Artificial Sequence
  source: /note=“Description of Artificial Sequence Synthetic peptide”
FTFSDYYMS

SEQ ID NO: 38 - protein, 17 aa, Artificial Sequence
  source: /note=“Description of Artificial Sequence Synthetic peptide”
YISSSGSTIYYADSVKG

SEQ ID NO: 39 - protein, 10 aa, Artificial Sequence
  source: /note=“Description of Artificial Sequence Synthetic peptide”
YISSSGSTIY

SEQ ID NO: 40 - protein, 15 aa, Artificial Sequence
  source: /note=“Description of Artificial Sequence Synthetic peptide”
GGRNRGDSSLSGIDV

SEQ ID NO: 41 - protein, 11 aa, Artificial Sequence
  source: /note=“Description of Artificial Sequence Synthetic peptide”
RASQSINSYLN

SEQ ID NO: 42 - protein, 7 aa, Artificial Sequence
  source: /note=“Description of Artificial Sequence Synthetic peptide”
AASSLQS

SEQ ID NO: 43 - protein, 8 aa, Artificial Sequence
  source: /note=“Description of Artificial Sequence Synthetic peptide”
QQQYSDIT

SEQ ID NO: 44 - protein, 124 aa, Artificial Sequence
  source: /note=“Description of Artificial Sequence Synthetic polypeptide”
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARGGRNRGDSSLSGIDVWGQGTTVTVSS

SEQ ID NO: 45 - protein, 106 aa, Artificial Sequence
  source: /note=“Description of Artificial Sequence Synthetic polypeptide”
DIQMTQSPSSLSASVGDRVTITCRASQSINSYLNWYQQKPGKAPKLLIYAASSLQSGVPSRFSGSGSGTDFTLTISSLQPEDFATYYCQQQYSDITFGGGTKVEIK

SEQ ID NO: 46 - protein, 15 aa, Artificial Sequence
  source: /note=“Description of Artificial Sequence Synthetic peptide”
GGPSRGDALAEYFQH

SEQ ID NO: 47 - protein, 7 aa, Artificial Sequence
  source: /note=“Description of Artificial Sequence Synthetic peptide”
GASTRAT

SEQ ID NO: 48 - protein, 10 aa, Artificial Sequence
  source: /note=“Description of Artificial Sequence Synthetic peptide”
QQLVNYPPIT

SEQ ID NO: 49 - protein, 124 aa, Artificial Sequence
  source: /note=“Description of Artificial Sequence Synthetic polypeptide”
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARGGPSRGDALAEYFQHWGQGTTVTVSS

SEQ ID NO: 50 - protein, 108 aa, Artificial Sequence
  source: /note=“Description of Artificial Sequence Synthetic polypeptide”
EIVMTQSPATLSVSPGERATLSCRASQSVSSNLAWYQQKPGQAPRLLIYGASTRATGIPARFSGSGSGTEFTLTISSLQSEDFAVYYCQQLVNYPPITFGGGTKVEIK

SEQ ID NO: 51 - protein, 5 aa, Artificial Sequence
  source: /note=“Description of Artificial Sequence Synthetic peptide”
DYSMN

SEQ ID NO: 52 - protein, 9 aa, Artificial Sequence
  source: /note=“Description of Artificial Sequence Synthetic peptide”
FTFYDYSMN

SEQ ID NO: 53 - protein, 17 aa, Artificial Sequence
  source: /note=“Description of Artificial Sequence Synthetic peptide”
YISSSSSTIYYADSVKG

SEQ ID NO: 54 - protein, 10 aa, Artificial Sequence
  source: /note=“Description of Artificial Sequence Synthetic peptide”
YISSSSSTIY

SEQ ID NO: 55 - protein, 14 aa, Artificial Sequence
  source: /note=“Description of Artificial Sequence Synthetic peptide”
GLWSTEVRYYYMDV

SEQ ID NO: 56 - protein, 9 aa, Artificial Sequence
  source: /note=“Description of Artificial Sequence Synthetic peptide”
QQSNAWPFT

SEQ ID NO: 57 - protein, 123 aa, Artificial Sequence
  source: /note=“Description of Artificial Sequence Synthetic polypeptide”
EVQLVESGGGLVQPGGSLRLSCAASGFTFYDYSMNWVRQAPGKGLEWVSYISSSSSTIYYADSVKGRFTISRDNAKNSLYLQMNSLRAEDTAVYYCARGLWSTEVRYYYMDVWGKGTTVTVSS

SEQ ID NO: 58 - protein, 107 aa, Artificial Sequence
  source: /note=“Description of Artificial Sequence Synthetic polypeptide”
EIVMTQSPATLSVSPGERATLSCRASQSVSSNLAWYQQKPGQAPRLLIYSASTRATGIPARFSGSGSGTEFTLTISSLQSEDFAVYYCQQSNAWPFTFGGGTKVEIK

SEQ ID NO: 59 - protein, 5 aa, Artificial Sequence
  source: /note=“Description of Artificial Sequence Synthetic peptide”
DYSMH

SEQ ID NO: 60 - protein, 9 aa, Artificial Sequence
  source: /note=“Description of Artificial Sequence Synthetic peptide”
FTFDDYSMH

SEQ ID NO: 61 - protein, 123 aa, Artificial Sequence
  source: /note=“Description of Artificial Sequence Synthetic polypeptide”
EVQLVESGGGLVQPGGSLRLSCAASGFTFDDYSMHWVRQAPGKGLEWVSYISSSGSTIYYADSVKGRFTISRDNAKNSLYLQMNSLRAEDTAVYYCARGLWSTEVRYYYMDVWGKGTTVTVSS

SEQ ID NO: 62 - protein, 5 aa, Artificial Sequence
  source: /note=“Description of Artificial Sequence Synthetic peptide”
EYSMF

SEQ ID NO: 63 - protein, 9 aa, Artificial Sequence
  source: /note=“Description of Artificial Sequence Synthetic peptide”
FTFGEYSMF

SEQ ID NO: 64 - protein, 123 aa, Artificial Sequence
  source: /note=“Description of Artificial Sequence Synthetic polypeptide”
EVQLVESGGGLVQPGGSLRLSCAASGFTFGEYSMFWVRQAPGKGLEWVSYISSSSSTIYYADSVKGRFTISRDNAKNSLYLQMNSLRAEDTAVYYCARGLWSTEVRYYYMDVWGKGTTVTVSS

SEQ ID NO: 65 - protein, 11 aa, Artificial Sequence
  source: /note=“Description of Artificial Sequence Synthetic peptide”
RASQSISSYLN

SEQ ID NO: 66 - protein, 7 aa, Artificial Sequence
  source: /note=“Description of Artificial Sequence Synthetic peptide”
GASSLQS

SEQ ID NO: 67 - protein, 8 aa, Artificial Sequence
  source: /note=“Description of Artificial Sequence Synthetic peptide”
QQQYDDIT

SEQ ID NO: 68 - protein, 106 aa, Artificial Sequence
  source: /note=“Description of Artificial Sequence Synthetic polypeptide”
DIQMTQSPSSLSASVGDRVTITCRASQSISSYLNWYQQKPGKAPKLLIYGASSLQSGVPSRFSGSGSGTDFTLTISSLQPEDFATYYCQQQYDDITFGGGTKVEIK

SEQ ID NO: 69 - protein, 8 aa, Artificial Sequence
  source: /note=“Description of Artificial Sequence Synthetic peptide”
QQQYIDIT

SEQ ID NO: 70 - protein, 106 aa, Artificial Sequence
  source: /note=“Description of Artificial Sequence Synthetic polypeptide”
DIQMTQSPSSLSASVGDRVTITCRASQSISSYLNWYQQKPGKAPKLLIYGASSLQSGVPSRFSGSGSGTDFTLTISSLQPEDFATYYCQQQYIDITFGGGTKVEIK

SEQ ID NO: 71 - protein, 10 aa, Artificial Sequence
  source: /note=“Description of Artificial Sequence Synthetic peptide”
QQLTNHPPIA

SEQ ID NO: 72 - protein, 108 aa, Artificial Sequence
  source: /note=“Description of Artificial Sequence Synthetic polypeptide”
EIVMTQSPATLSVSPGERATLSCRASQSVSSNLAWYQQKPGQAPRLLIYGASTRATGIPARFSGSGSGTEFTLTISSLQSEDFAVYYCQQLTNHPPIAFGGGTKVEIK

SEQ ID NO: 73 - protein, 16 aa, Artificial Sequence
  source: /note=“Description of Artificial Sequence Synthetic peptide”
ERGNRGDTPRYYYMDV

SEQ ID NO: 74 - protein, 11 aa, Artificial Sequence
  source: /note=“Description of Artificial Sequence Synthetic peptide”
RASQSISRYLN

SEQ ID NO: 75 - protein, 9 aa, Artificial Sequence
  source: /note=“Description of Artificial Sequence Synthetic peptide”
QQSLVTPFT

SEQ ID NO: 76 - protein, 125 aa, Artificial Sequence
  source: /note=“Description of Artificial Sequence Synthetic polypeptide”
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARERGNRGDTPRYYYMDVWGKGTTVTVSS

SEQ ID NO: 77 - protein, 107 aa, Artificial Sequence
  source: /note=“Description of Artificial Sequence Synthetic polypeptide”
DIQMTQSPSSLSASVGDRVTITCRASQSISRYLNWYQQKPGKAPKLLIYAASSLQSGVPSRFSGSGSGTDFTLTISSLQPEDFATYYCQQSLVTPFTFGGGTKVEIK

SEQ ID NO: 78 - protein, 18 aa, Artificial Sequence
  source: /note=“Description of Artificial Sequence Synthetic peptide”
DRSGIAGRRWVYYYGMDV

SEQ ID NO: 79 - protein, 11 aa, Artificial Sequence
  source: /note=“Description of Artificial Sequence Synthetic peptide”
RASQSVSSYLA

SEQ ID NO: 80 - protein, 7 aa, Artificial Sequence
  source: /note=“Description of Artificial Sequence Synthetic peptide”
DASNRAT

SEQ ID NO: 81 - protein, 9 aa, Artificial Sequence
  source: /note=“Description of Artificial Sequence Synthetic peptide”
QQRSNLPYT

SEQ ID NO: 82 - protein, 127 aa, Artificial Sequence
  source: /note=“Description of Artificial Sequence Synthetic polypeptide”
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARDRSGIAGRRWVYYYGMDVWGQGTTVTVSS

SEQ ID NO: 83 - protein, 107 aa, Artificial Sequence
  source: /note=“Description of Artificial Sequence Synthetic polypeptide”
EIVLTQSPATLSLSPGERATLSCRASQSVSSYLAWYQQKPGQAPRLLIYDASNRATGIPARFSGSGSGTDFTLTISSLEPEDFAVYYCQQRSNLPYTFGGGTKVEIK

SEQ ID NO: 84 - protein, 5 aa, Artificial Sequence
  source: /note=“Description of Artificial Sequence Synthetic peptide”
SYWIG

SEQ ID NO: 85 - protein, 9 aa, Artificial Sequence
  source: /note=“Description of Artificial Sequence Synthetic peptide”
YSFTSYWIG

SEQ ID NO: 86 - protein, 17 aa, Artificial Sequence
  source: /note=“Description of Artificial Sequence Synthetic peptide”
IIYPGDSDTRYSPSFQG

SEQ ID NO: 87 - protein, 10 aa, Artificial Sequence
  source: /note=“Description of Artificial Sequence Synthetic peptide”
IIYPGDSDTR

SEQ ID NO: 88 - protein, 17 aa, Artificial Sequence
  source: /note=“Description of Artificial Sequence Synthetic peptide”
GPRSRGDGPSNYYYMDV

SEQ ID NO: 89 - protein, 11 aa, Artificial Sequence
  source: /note=“Description of Artificial Sequence Synthetic peptide”
RASQSISSWLA

SEQ ID NO: 90 - protein, 7 aa, Artificial Sequence
  source: /note=“Description of Artificial Sequence Synthetic peptide”
KASSLES

SEQ ID NO: 91 - protein, 9 aa, Artificial Sequence
  source: /note=“Description of Artificial Sequence Synthetic peptide”
QQYHSFSFT

SEQ ID NO: 92 - protein, 126 aa, Artificial Sequence
  source: /note=“Description of Artificial Sequence Synthetic polypeptide”
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARGPRSRGDGPSNYYYMDVWGQGTLVTVSS

SEQ ID NO: 93 - protein, 107 aa, Artificial Sequence
  source: /note=“Description of Artificial Sequence Synthetic polypeptide”
DIQMTQSPSTLSASVGDRVTITCRASQSISSWLAWYQQKPGKAPKLLIYKASSLESGVPSRFSGSGSGTEFTLTISSLQPDDFATYYCQQYHSFSFTFGGGTKVEIK

SEQ ID NO: 94 - protein, 9 aa, Artificial Sequence
  source: /note=“Description of Artificial Sequence Synthetic peptide”
QQYRPLPPT

SEQ ID NO: 95 - protein, 107 aa, Artificial Sequence
  source: /note=“Description of Artificial Sequence Synthetic polypeptide”
DIQMTQSPSTLSASVGDRVTITCRASQSISSWLAWYQQKPGKAPKLLIYKASSLESGVPSRFSGSGSGTEFTLTISSLQPDDFATYYCQQYRPLPPTFGGGTKVEIK

SEQ ID NO: 96 - protein, 5 aa, Artificial Sequence
  source: /note=“Description of Artificial Sequence Synthetic peptide”
SFYMH

SEQ ID NO: 97 - protein, 9 aa, Artificial Sequence
  source: /note=“Description of Artificial Sequence Synthetic peptide”
YTFRSFYMH

SEQ ID NO: 98 - protein, 17 aa, Artificial Sequence
  source: /note=“Description of Artificial Sequence Synthetic peptide”
VINPSLGSTGYAQKFQG

SEQ ID NO: 99 - protein, 10 aa, Artificial Sequence
  source: /note=“Description of Artificial Sequence Synthetic peptide”
VINPSLGSTG

SEQ ID NO: 100 - protein, 122 aa, Artificial Sequence
  source: /note=“Description of Artificial Sequence Synthetic polypeptide”
QVQLVQSGAEVKKPGASVKVSCKASGYTFRSFYMHWVRQAPGQGLEWMGVINPSLGSTGYAQKFQGRVTMTRDTSTSTVYMELSSLRSEDTAVYYCARETNYYRGGPAFDIWGQGTMVTVSS

SEQ ID NO: 101 - protein, 17 aa, Artificial Sequence
  source: /note=“Description of Artificial Sequence Synthetic peptide”
KSSQSVLYSSNNKNYLA

SEQ ID NO: 102 - protein, 7 aa, Artificial Sequence
  source: /note=“Description of Artificial Sequence Synthetic peptide”
WASTRES

SEQ ID NO: 103 - protein, 9 aa, Artificial Sequence
  source: /note=“Description of Artificial Sequence Synthetic peptide”
QQYVAFPRT

SEQ ID NO: 104 - protein, 113 aa, Artificial Sequence
  source: /note=“Description of Artificial Sequence Synthetic polypeptide”
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYVAFPRTFGGGTKVEIK

SEQ ID NO: 105 - protein, 11 aa, Artificial Sequence
  source: /note=“Description of Artificial Sequence Synthetic peptide”
RASQSVSRYLA

SEQ ID NO: 106 - protein, 10 aa, Artificial Sequence
  source: /note=“Description of Artificial Sequence Synthetic peptide”
QQLSLHPPYT

SEQ ID NO: 107 - protein, 108 aa, Artificial Sequence
  source: /note=“Description of Artificial Sequence Synthetic polypeptide”
EIVLTQSPATLSLSPGERATLSCRASQSVSRYLAWYQQKPGQAPRLLIYDASNRATGIPARFSGSGSGTDFTLTISSLEPEDFAVYYCQQLSLHPPYTFGGGTKVEIK

SEQ ID NO: 108 - protein, 4 aa, Artificial Sequence
  source: /note=“Description of Artificial Sequence Synthetic peptide”
GGGS

SEQ ID NO: 109 - protein, 4 aa, Artificial Sequence
  source: /note=“Description of Artificial Sequence Synthetic peptide”
SGGG

SEQ ID NO: 110 - protein, 5 aa, Artificial Sequence
  source: /note=“Description of Artificial Sequence Synthetic peptide”
  source: /note=“See specification as filed for detailed description of substitutions and preferred embodiments”
GGGGS

SEQ ID NO: 111 - protein, 5 aa, Artificial Sequence
  source: /note=“Description of Artificial Sequence Synthetic peptide”
  source: /note=“See specification as filed for detailed description of substitutions and preferred embodiments”
SGGGG

SEQ ID NO: 112 - protein, 6 aa, Artificial Sequence
  source: /note=“Description of Artificial Sequence Synthetic peptide”
GGGGGS

SEQ ID NO: 113 - protein, 6 aa, Artificial Sequence
  source: /note=“Description of Artificial Sequence Synthetic peptide”
SGGGGG

SEQ ID NO: 114 - protein, 7 aa, Artificial Sequence
  source: /note=“Description of Artificial Sequence Synthetic peptide”
GGGGGGS

SEQ ID NO: 115 - protein, 7 aa, Artificial Sequence
  source: /note=“Description of Artificial Sequence Synthetic peptide”
SGGGGGG

SEQ ID NO: 116 - protein, 788 aa, Homo sapiens
MGIELLCLFFLFLGRNDHVQGGCALGGAETCEDCLLIGPQCAWCAQENFTHPSGVGERCDTPANLLAKGCQLNFIENPVSQVEILKNKPLSVGRQKNSSDIVQIAPQSLILKLRPGGAQTLQVHVRQTEDYPVDLYYLMDLSASMDDDLNTIKELGSRLSKEMSKLTSNFRLGFGSFVEKPVSPFVKTTPEEIANPCSSIPYFCLPTFGFKHILPLTNDAERFNEIVKNQKISANIDTPEGGFDAIMQAAVCKEKIGWRNDSLHLLVFVSDADSHFGMDSKLAGIVIPNDGLCHLDSKNEYSMSTVLEYPTIGQLIDKLVQNNVLLIFAVTQEQVHLYENYAKLIPGATVGLLQKDSGNILQLIISAYEELRSEVELEVLGDTEGLNLSFTAICNNGTLFQHQKKCSHMKVGDTASFSVTVNIPHCERRSRHIIIKPVGLGDALELLVSPECNCDCQKEVEVNSSKCHHGNGSFQCGVCACHPGHMGPRCECGEDMLSTDSCKEAPDHPSCSGRGDCYCGQCICHLSPYGNIYGPYCQCDNFSCVRHKGLLCGGNGDCDCGECVCRSGWTGEYCNCTTSTDSCVSEDGVLCSGRGDCVCGKCVCTNPGASGPTCERCPTCGDPCNSKRSCIECHLSAAGQAREECVDKCKLAGATISEEEDFSKDGSVSCSLQGENECLITFLITTDNEGKTIIHSINEKDCPKPPNIPMIMLGVSLAILLIGVVLLCIWKLLVSFHDRKEVAKFEAERSKAKWQTGTNPLYRGSTSTFKNVTYKHREKQKVDLSTDC

SEQ ID NO: 117 - protein, 963 aa, Artificial Sequence
  source: /note=“Description of Artificial Sequence Synthetic polypeptide”
FNLDVDSPAEYSGPEGSYFGFAVDFFVPSASSRMFLLVGAPKANTTQPGIVEGGQVLKCDWSSTRRCQPIEFDATGNRDYAKDDPLEFKSHQWFGASVRSKQDKILACAPLYHWRTEMKQEREPVGTCFLQDGTKTVEYAPCRSQDIDADGQGFCQGGFSIDFTKADRVLLGGPGSFYWQGQLISDQVAEIVSKYDPNVYSIKYNNQLATRTAQAIFDDSYLGYSVAVGDFNGDGIDDFVSGVPRAARTLGMVYIYDGKNMSSLYNFTGEQMAAYFGFSVAATDINGDDYADVFIGAPLFMDRGSDGKLQEVGQVSVSLQRASGDFQTTKLNGFEVFARFGSAIAPLGDLDQDGFNDIAIAAPYGGEDKKGIVYIFNGRSTGLNAVPSQILEGQWAARSMPPSFGYSMKGATDIDKNGYPDLIVGAFGVDRAILYRARPVITVNAGLEVYPSILNQDNKTCSLPGTALKVSCFNVRFCLKADGKGVLPRKLNFQVELLLDKLKQKGAIRRALFLYSRSPSHSKNMTISRGGLMQCEELIAYLRDESEFRDKLTPITIFMEYRLDYRTAADTTGLQPILNQFTPANISRQAHILLDCGEDNVCKPKLEVSVDSDQKKIYIGDDNPLTLIVKAQNQGEGAYEAELIVSIPLQADFIGVVRNNEALARLSCAFKTENQTRQVVCDLGNPMKAGTQLLAGLRFSVHQQSEMDTSVKFDLQIQSSNLFDKVSPVVSHKVDLAVLAAVEIRGVSSPDHVFLPIPNWEHKENPETEEDVGPVVQHIYELRNNGPSSFSKAMLHLQWPYKYNNNTLLYILHYDIDGPMNCTSDMEINPLRIKISSLQTTEKNDTVAGQGERDHLITKRDLALSEGDIHTLGCGVAQCLKIVCQVGRLDRGKSAILYVKSLLWTETFMNKENQNHSYSLKSSASFNVIEFPYKNLPIEDITNSTLVTTNVTWGIQPAPMPVP

SEQ ID NO: 118 - protein, 708 aa, Artificial Sequence
  source: /note=“Description of Artificial Sequence Synthetic polypeptide”
QTDENRCLKANAKSCGECIQAGPNCGWCTNSTFLQEGMPTSARCDDLEALKKKGCPPDDIENPRGSKDIKKNKNVTNRSKGTAEKLKPEDITQIQPQQLVLRLRSGEPQTFTLKFKRAEDYPIDLYYLMDLSYSMKDDLENVKSLGTDLMNEMRRITSDFRIGFGSFVEKTVMPYISTTPAKLRNPCTSEQNCTSPFSYKNVLSLTNKGEVFNELVGKQRISGNLDSPEGGFDAIMQVAVCGSLIGWRNVTRLLVFSTDAGFHFAGDGKLGGIVLPNDGQCHLENNMYTMSHYYDYPSIAHLVQKLSENNIQTIFAVTEEFQPVYKELKNLIPKSAVGTLSANSSNVIQLIIDAYNSLSSEVILENGKLSEGVTISYKSYCKNGVNGTGENGRKCSNISIGDEVQFEISITSNKCPKKDSDSFKIRPLGFTEEVEVILQYICECECQSEGIPESPKCHEGNGTFECGACRCNEGRVGRHCECSTDEVNSEDMDAYCRKENSSEICSNNGECVCGQCVCRKRDNTNEIYSGKFCECDNFNCDRSNGLICGGNGVCKCRVCECNPNYTGSACDCSLDTSTCEASNGQICNGRGICECGVCKCTDPKFQGQTCEMCQTCLGVCAEHKECVQCRAFNKGEKKDTCTQECSYFNITKVESRDKLPQPVQPDPVSHCKEKDVDDCWFYFTYSVNGNNEVMVHVVENPECPTGPD

SEQ ID NO: 119 - protein, 953 aa, Artificial Sequence
  source: /note=“Description of Artificial Sequence Synthetic polypeptide”
QTDENRCLKANAKSCGECIQAGPNCGWCTNSTFLQEGMPTSARCDDLEALKKKGCPPDDIENPRGSKDIKKNKNVTNRSKGTAEKLKPEDITQIQPQQLVLRLRSGEPQTFTLKFKRAEDYPIDLYYLMDLSYSMKDDLENVKSLGTDLMNEMRRITSDFRIGFGSFVEKTVMPYISTTPAKLRNPCTSEQNCTSPFSYKNVLSLTNKGEVFNELVGKQRISGNLDSPEGGFDAIMQVAVCGSLIGWRNVTRLLVFSTDAGFHFAGDGKLGGIVLPNDGQCHLENNMYTMSHYYDYPSIAHLVQKLSENNIQTIFAVTEEFQPVYKELKNLIPKSAVGTLSANSSNVIQLIIDAYNSLSSEVILENGKLSEGVTISYKSYCKNGVNGTGENGRKCSNISIGDEVQFEISITSNKCPKKDSDSFKIRPLGFTEEVEVILQYICECECQSEGIPESPKCHEGNGTFECGACRCNEGRVGRHCECSTDEVNSEDMDAYCRKENSSEICSNNGECVCGQCVCRKRDNTNEIYSGKFCECDNFNCDRSNGLICGGNGVCKCRVCECNPNYTGSACDCSLDTSTCEASNGQICNGRGICECGVCKCTDPKFQGQTCEMCQTCLGVCAEHKECVQCRAFNKGEKKDTCTQECSYFNITKVESRDKLPQPVQPDPVSHCKEKDVDDCWFYFTYSVNGNNEVMVHVVENPECPTGPDDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGLNDIFEAQKIEWHE

SEQ ID NO: 120 - protein, 1015 aa, Artificial Sequence
  source: /note=“Description of Artificial Sequence Synthetic polypeptide”
FNLDVDSPAEYSGPEGSYFGFAVDFFVPSASSRMFLLVGAPKANTTQPGIVEGGQVLKCDWSSTRRCQPIEFDATGNRDYAKDDPLEFKSHQWFGASVRSKQDKILACAPLYHWRTEMKQEREPVGTCFLQDGTKTVEYAPCRSQDIDADGQGFCQGGFSIDFTKADRVLLGGPGSFYWQGQLISDQVAEIVSKYDPNVYSIKYNNQLATRTAQAIFDDSYLGYSVAVGDFNGDGIDDFVSGVPRAARTLGMVYIYDGKNMSSLYNFTGEQMAAYFGFSVAATDINGDDYADVFIGAPLFMDRGSDGKLQEVGQVSVSLQRASGDFQTTKLNGFEVFARFGSAIAPLGDLDQDGFNDIAIAAPYGGEDKKGIVYIFNGRSTGLNAVPSQILEGQWAARSMPPSFGYSMKGATDIDKNGYPDLIVGAFGVDRAILYRARPVITVNAGLEVYPSILNQDNKTCSLPGTALKVSCFNVRFCLKADGKGVLPRKLNFQVELLLDKLKQKGAIRRALFLYSRSPSHSKNMTISRGGLMQCEELIAYLRDESEFRDKLTPITIFMEYRLDYRTAADTTGLQPILNQFTPANISRQAHILLDCGEDNVCKPKLEVSVDSDQKKIYIGDDNPLTLIVKAQNQGEGAYEAELIVSIPLQADFIGVVRNNEALARLSCAFKTENQTRQVVCDLGNPMKAGTQLLAGLRFSVHQQSEMDTSVKFDLQIQSSNLFDKVSPVVSHKVDLAVLAAVEIRGVSSPDHVFLPIPNWEHKENPETEEDVGPVVQHIYELRNNGPSSFSKAMLHLQWPYKYNNNTLLYILHYDIDGPMNCTSDMEINPLRIKISSLQTTEKNDTVAGQGERDHLITKRDLALSEGDIHTLGCGVAQCLKIVCQVGRLDRGKSAILYVKSLLWTETFMNKENQNHSYSLKSSASFNVIEFPYKNLPIEDITNSTLVTTNVTWGIQPAPMPVPTGGLENLYFQGGENAQCEKELQALEKENAQLEWELQALEKELAQWSHPQFEK

SEQ ID NO: 121 - protein, 794 aa, Artificial Sequence
  source: /note=“Description of Artificial Sequence Synthetic polypeptide”
QTDENRCLKANAKSCGECIQAGPNCGWCTNSTFLQEGMPTSARCDDLEALKKKGCPPDDIENPRGSKDIKKNKNVTNRSKGTAEKLKPEDITQIQPQQLVLRLRSGEPQTFTLKFKRAEDYPIDLYYLMDLSYSMKDDLENVKSLGTDLMNEMRRITSDFRIGFGSFVEKTVMPYISTTPAKLRNPCTSEQNCTSPFSYKNVLSLTNKGEVFNELVGKQRISGNLDSPEGGFDAIMQVAVCGSLIGWRNVTRLLVFSTDAGFHFAGDGKLGGIVLPNDGQCHLENNMYTMSHYYDYPSIAHLVQKLSENNIQTIFAVTEEFQPVYKELKNLIPKSAVGTLSANSSNVIQLIIDAYNSLSSEVILENGKLSEGVTISYKSYCKNGVNGTGENGRKCSNISIGDEVQFEISITSNKCPKKDSDSFKIRPLGFTEEVEVILQYICECECQSEGIPESPKCHEGNGTFECGACRCNEGRVGRHCECSTDEVNSEDMDAYCRKENSSEICSNNGECVCGQCVCRKRDNTNEIYSGKFCECDNFNCDRSNGLICGGNGVCKCRVCECNPNYTGSACDCSLDTSTCEASNGQICNGRGICECGVCKCTDPKFQGQTCEMCQTCLGVCAEHKECVQCRAFNKGEKKDTCTQECSYFNITKVESRDKLPQPVQPDPVSHCKEKDVDDCWFYFTYSVNGNNEVMVHVVENPECPTGPDTSGLENLYFQGGKNAQCKKKLQALKKKNAQLKWKLQALKKKLAQGGHHHHHHGGGGSGGGGSGGGGSGGGGSLNDIFEAQKIEWHE

SEQ ID NO: 122 - protein, 6 aa, Artificial Sequence
  source: /note=“Description of Artificial Sequence Synthetic 6xHis tag”
HHHHHH

(19) United States
(12) Patent Application Publication
(10) Pub. No.: US 2022/0195052 A1
(43) Pub. Date: Jun. 23, 2022

(54) ANTI-INTEGRIN ANTIBODIES AND USES THEREOF

(71) Applicant: Biogen MA Inc., Cambridge, MA (US)

(72) Inventors: Christilyn Graff, Cambridge, MA (US); Christina Palmer, Waltham, MA (US); Brett Blakeley, Framingham, MA (US); Tracey Mullen, Quincy, MA (US); Agnes Gardet, La Jolla, CA (US)

(21) Appl. No.: 17/599,716

(22) PCT Filed: Apr. 8, 2020
(86) PCT No.: PCT/US20/27271

§ 371 Date: Sep. 29, 2021

Related U.S. Application Data
(60) Provisional application No. 62/830,961, filed on Apr. 8, 2019.

(51) Int. Cl. C07K 16/28

(52) U.S. Cl. CPC C07K 16/2842; C07K 2317/21; C07K 2317/92; C07K 2317/76; C07K 2317/33

(57) ABSTRACT

Anti-integrin antibodies are disclosed. Also disclosed are methods of using the antibodies to treat or prevent disorders such as fibrotic diseases, cancer, ophthalmology disorders, and NAFLD. Further disclosed are methods of selecting an antibody that specifically binds to αvβ\ddot{í}, or that binds to αvβ\ddot{í} and αvβó, or that binds to one or more members of the RGD sub-family of integrins.

### CROSS-REFERENCE TO RELATED APPLICATION

This application claims the benefit of U.S. Provisional Application No. 62/830,961 filed Apr. 8, 2019, the content of which is incorporated by reference herein in its entirety.

### TECHNICAL FIELD

This invention relates generally to anti-integrin antibodies (e.g., antibodies that bind to one or more members of the RGD sub-family of integrins) and uses thereof.

### BACKGROUND

Integrins are cell adhesion receptors that play important roles during developmental and pathological processes. Integrins are widely expressed, and every nucleated cell in the body possesses a specific integrin signature. These receptors are composed of non-covalently associated alpha (α) and beta (β) chains that combine to give a variety of heterodimeric proteins with distinct cellular and adhesive specificities. The integrin family is composed of 24 αβ heterodimeric members that mediate the attachment of cells to the extracellular matrix (ECM) but that also take part in specialized cell-cell interactions. The α and β subunits show no homology to each other, but different a subunits have similarities among themselves, and there are conserved regions in the different integrin β subunits. A subset of integrins (8 out of 24) recognizes the RGD sequence (arginine (R), glycine (G) and aspartic acid (D)) in the native ligands, and are also referred to as RGD-binding integrins, which include αvβ1, αvβ3, αvβ5, αvβ6, αvβ8, α5β1, α8β1, and αIIBβ3 integrins. Integrins have been implicated in the regulation of a variety of cellular processes including cellular adhesion, migration, invasion, differentiation, proliferation, apoptosis, and gene expression. Accordingly, there is a need to develop anti-integrin antibodies that are useful in the treatment of diseases involved in the integrin pathway, such as fibrotic diseases, ophthalmology diseases, and cancer.

### SUMMARY

In one aspect, this disclosure features an antibody that specifically binds to αvβ1 integrin but not to other integrins. In some embodiments, the antibodies do not bind other αv- or β1-containing integrin heterodimers. In some embodiments, the anti-αvβ1 antibodies do not bind other RGD integrins (e.g., αvβ3, αvβ5, αvβ6, αvβ8, α501, α8β1, and αIIBβ3 integrins). In some embodiments, the antibody competes with and/or binds the same epitope as a reference anti-αvβ1 integrin antibody comprising a heavy chain variable region (VH) and a light chain variable region (VL), wherein the VH and VL of the reference antibody comprise: (i) the amino acid sequence set forth in SEQ ID NO:11 and the amino acid sequence set forth in SEQ ID NO:12, respectively; (ii) the amino acid sequence set forth in SEQ ID NO:21 and the amino acid sequence set forth in SEQ ID NO:22, respectively; (iii) the amino acid sequence set forth in SEQ ID NO:27 and the amino acid sequence set forth in SEQ ID NO:28, respectively; (iv) the amino acid sequence set forth in SEQ ID NO:30 and the amino acid sequence set forth in SEQ ID NO:12, respectively; (v) the amino acid sequence set forth in SEQ ID NO:35 and the amino acid sequence set forth in SEQ ID NO:22, respectively; (vi) the amino acid sequence set forth in SEQ ID NO:44 and the amino acid sequence set forth in SEQ ID NO:45, respectively; (vii) the amino acid sequence set forth in SEQ ID NO:49 and the amino acid sequence set forth in SEQ ID NO:50, respectively; (viii) the amino acid sequence set forth in SEQ ID NO:57 and the amino acid sequence set forth in SEQ ID NO:58, respectively; (ix) the amino acid sequence set forth in SEQ ID NO:61 and the amino acid sequence set forth in SEQ ID NO:58, respectively; or (x) the amino acid sequence set forth in SEQ ID NO:64 and the amino acid sequence set forth in SEQ ID NO:58, respectively.

In another aspect, this disclosure features an antibody that specifically binds to both αvβ1 and αvβ6 integrins but not to other integrins. In some embodiments, the antibodies do not bind other αv-, β1, or β6-containing integrin heterodimers. In some instances, the antibody does not bind to RGD-binding integrins (e.g. αvβ3, αvβ5, αvβ8, α5β1, α8β1, and αIIBβ3) other than αvβ1 and αvβ6 integrins. In some embodiments, the antibody competes with and/or binds the same epitope as a reference antibody that binds both αvβ1 and αvβ6 integrins and comprises a heavy chain variable region (VH) and a light chain variable region (VL), wherein the VH and VL of the reference antibody comprise: (i) the amino acid sequence set forth in SEQ ID NO:44 and the amino acid sequence set forth in SEQ ID NO:68, respectively; (ii) the amino acid sequence set forth in SEQ ID NO:44 and the amino acid sequence set forth in SEQ ID NO:70, respectively; (iii) the amino acid sequence set forth in SEQ ID NO:49 and the amino acid sequence set forth in SEQ ID NO:72, respectively; or (iv) the amino acid sequence set forth in SEQ ID NO:76 and the amino acid sequence set forth in SEQ ID NO:77, respectively.

In another aspect, this disclosure features an antibody that specifically binds to αvβ1 and one or more integrins selected from the group consisting of αvβ3, αvβ5, αvβ6, αvβ8, α5β1, α8β1, and αIIBβ3. In some embodiments, the antibody binds αvβ1 and αvβ8. In some embodiments, the antibody binds αvβ1 and αvβ3. In some embodiments, the antibody binds αvβ1, αvβ3, αvβ5, αvβ6, and αvβ8. In some embodiments, the antibodies do not bind to integrins other than one or more of the RGD-binding integrins. In some embodiments, the antibody competes with and/or binds the same epitope as a reference antibody comprising a heavy chain variable region (VH) and a light chain variable region (VL), wherein the VH and VL of the reference antibody comprise: (i) the amino acid sequence set forth in SEQ ID NO:82 and the amino acid sequence set forth in SEQ ID NO:83, respectively; (ii) the amino acid sequence set forth in SEQ ID NO:92 and the amino acid sequence set forth in SEQ ID NO:93, respectively; (iii) the amino acid sequence set forth in SEQ ID NO:92 and the amino acid sequence set forth in SEQ ID NO:95, respectively; (iv) the amino acid sequence set forth in SEQ ID NO:100 and the amino acid sequence set forth in SEQ ID NO:28, respectively; (v) the amino acid sequence set forth in SEQ ID NO:21 and the amino acid sequence set forth in SEQ ID NO:104, respectively; or (vi) the amino acid sequence set forth in SEQ ID NO:49 and the amino acid sequence set forth in SEQ ID NO:107, respectively.

In another aspect, this disclosure features an antibody that specifically binds to human αvβ1 and has one or more (e.g., 1, 2, 3, 4 or 5) of the following properties: (i) binds with high affinity of KD≤20 nM (bivalent affinity) to human αvβ1; (ii) blocks αvβ1 interaction with its ligand (e.g., LAP and fibronectin); (iii) is cation-dependent (e.g., calcium and magnesium; or manganese) or cation-independent for binding to human αvβ1; (iv) binds to αvβ1 on fibroblasts; and (v) inhibits fibroblast TGFβ response (e.g., as assessed by a LPA-induced PAI-1 assay). In some embodiments, the antibody is internalized. In some embodiments, the antibody binds to cynomolgus monkey, mouse, and rat αvβ1. In some embodiments, the antibody comprises the VH CDR1, VH CDR2, and VH CDR3 of Exemplary Antibodies 1-10. In some embodiments, the antibody comprises the VL CDR1, VL CDR2, and VL CDR3 of Exemplary Antibodies 1-10. In some embodiments, the antibody competes with and/or binds the same epitope as a reference anti-αvβ1 integrin antibody comprising the VH and VL of Exemplary Antibodies 1-10.

In another aspect, this disclosure features an antibody that specifically binds to both human αvβ1 and human αvβ6 and has one or more (e.g., 1, 2, 3, 4 or 5) of the following properties: (i) bind with high affinity of KD≤20 nM (bivalent affinity) to human αvβ1, and with affinity of 100 nM (bivalent affinity) to human αvβ6; (ii) blocks αvβ1 and/or αvβ6 interaction with its ligand (e.g., LAP and fibronectin); (iii) is cation-dependent (e.g., calcium and magnesium; or manganese) for binding to human αvβ1 and/or αvβ6; (iv) binds to αvβ1 on fibroblasts; and (v) inhibits fibroblast TGFβ response (e.g., as assessed by a LPA-induced PAI-1 assay). In some embodiments, the antibody comprises the VH CDR1, VH CDR2, and VH CDR3 of Exemplary Antibodies 11-14. In some embodiments, the antibody comprises the VL CDR1, VL CDR2, and VL CDR3 of Exemplary Antibodies 11-14. In some embodiments, the antibody competes with and/or binds the same epitope as a reference antibody that binds both αvβ1 and αvβ6 integrins and comprises the VH and VL of Exemplary Antibodies 11-14.

In another aspect, this disclosure features an antibody that specifically binds to both human αvβ1 and one or more of the other RGD-binding integrins and has one or more (e.g., 1, 2, 3, 4 or 5) of the following properties: (i) which bind with high affinity of KD≤20 nM (bivalent affinity) to human αvβ1, and with affinity of 100 nM (bivalent affinity) to other RGD binding integrins; (ii) blocks αvβ1 and/or RGD family integrin interaction with its ligand (e.g., LAP and fibronectin); (iii) is cation-dependent (e.g., calcium and magnesium; or manganese) or cation-independent for binding to human αvβ1 and/or RGD binding integrins; (iv) binds to αvβ1 and/or RGD binding integrins on fibroblasts; and (v) inhibits fibroblast TGFβ response (e.g., as assessed by a LPA-induced PAI-1 assay). In some embodiments, the antibody is internalized. In some embodiments, the antibody binds to cynomolgus monkey, mouse, and rat αvβ1. In some embodiments, the antibody comprises the VH CDR1, VH CDR2, and VH CDR3 of Exemplary Antibodies 15-20.

In some embodiments, the antibody comprises the VL CDR1, VL CDR2, and VL CDR3 of Exemplary Antibodies 15-20. In some embodiments, the antibody competes with and/or binds the same epitope as a reference antibody comprising the VH and VL of Exemplary Antibodies 15-20.

In another aspect, this disclosure features an antibody that specifically binds to both human αvβ1 and/or one or more of the other RGD-binding integrins and has one or more (e.g., 1, 2, 3, 4, 5, 6, or 7) of the following properties: (i) which bind with high affinity of KD≤20 nM (bivalent affinity) to human αvβ1, and (if it also binds other RGD family integrins) with affinity of 100 nM (bivalent affinity) to other RGD binding integrins; (ii) blocks αvβ1 and/or RGD family integrin interaction with its ligand (e.g., LAP and fibronectin); (iii) is cation-dependent (e.g., calcium and magnesium; or manganese) or cation-independent for binding to human αvβ1 and/or RGD binding integrins; (iv) binds to αvβ1 and/or RGD binding integrins on fibroblasts; (v) inhibits fibroblast TGFβ response (e.g., as assessed by a LPA-induced PAI-1 assay); (vi) is internalized; (vii) binds to cynomolgus monkey, mouse, and rat αvβ1.

In another aspect, this disclosure features an antibody that binds to αvβ1 integrin but not to other integrins (e.g., other RGD-family integrins). The antibody comprises a VH comprising VHCDR1, VHCDR2, and VHCDR3, and a VL comprising VLCDR1, VLCDR2, and VLCDR3, wherein VHCDR1, VHCDR2, VHCDR3, VLCDR1, VLCDR2, and VLCDR3 comprise: (i) SEQ ID NOs:4, 6, 7, 8, 9, and 10, respectively; (ii) SEQ ID NOs:14, 16, 17, 18, 19, and 20, respectively; (iii) SEQ ID NOs:4, 6, 23, 24, 25, and 26, respectively; (iv) SEQ ID NOs:29, 6, 7, 8, 9, and 10, respectively; (v) SEQ ID NOs:32, 34, 17, 18, 19, and 20, respectively; (vi) SEQ ID NOs:37, 39, 40, 41, 42, and 43, respectively; (vii) SEQ ID NOs:37, 39, 46, 18, 47, and 48, respectively; (viii) SEQ ID NOs:52, 54, 55, 18, 19, and 56, respectively; (ix) SEQ ID NOs:60, 39, 55, 18, 19, and 56, respectively; or (x) SEQ ID NOs:63, 54, 55, 18, 19, and 56, respectively.

In some embodiments of the above aspect, the anti-αvβ1 antibody comprises (i) a VH that is at least 75%, 80%, 85%, 90%, 91%, 92%, 93%, 94%, 95%, 96%, 97%, 98%, 99%, or 100% identical to the amino acid sequences set forth in SEQ ID NO:11, and a VL that is at least 75%, 80%, 85%, 90%, 91%, 92%, 93%, 94%, 95%, 96%, 97%, 98%, 99%, or 100% identical to the amino acid sequences set forth in SEQ ID NO:12; (ii) a VH that is at least 75%, 80%, 85%, 90%, 91%, 92%, 93%, 94%, 95%, 96%, 97%, 98%, 99%, or 100% identical to the amino acid sequences set forth in SEQ ID NO:21, and a VL that is at least 75%, 80%, 85%, 90%, 91%, 92%, 93%, 94%, 95%, 96%, 97%, 98%, 99%, or 100% identical to the amino acid sequences set forth in SEQ ID NO:22; (iii) a VH that is at least 75%, 80%, 85%, 90%, 91%, 92%, 93%, 94%, 95%, 96%, 97%, 98%, 99%, or 100% identical to the amino acid sequences set forth in SEQ ID NO:27, and a VL that is at least 75%, 80%, 85%, 90%, 91%, 92%, 93%, 94%, 95%, 96%, 97%, 98%, 99%, or 100% identical to the amino acid sequences set forth in SEQ ID NO:28; (iv) a VH that is at least 75%, 80%, 85%, 90%, 91%, 92%, 93%, 94%, 95%, 96%, 97%, 98%, 99%, or 100% identical to the amino acid sequences set forth in SEQ ID NO:30, and a VL that is at least 75%, 80%, 85%, 90%, 91%, 92%, 93%, 94%, 95%, 96%, 97%, 98%, 99%, or 100% identical to the amino acid sequences set forth in SEQ ID NO:12; (v) a VH that is at least 75%, 80%, 85%, 90%, 91%, 92%, 93%, 94%, 95%, 96%, 97%, 98%, 99%, or 100% identical to the amino acid sequences set forth in SEQ ID NO:35, and a VL that is at least 75%, 80%, 85%, 90%, 91%, 92%, 93%, 94%, 95%, 96%, 97%, 98%, 99%, or 100% identical to the amino acid sequences set forth in SEQ ID NO:22; (vi) a VH that is at least 75%, 80%, 85%, 90%, 91%, 92%, 93%, 94%, 95%, 96%, 97%, 98%, 99%, or 100% identical to the amino acid sequences set forth in SEQ ID NO:44, and a VL that is at least 75%, 80%, 85%, 90%, 91%, 92%, 93%, 94%, 95%, 96%, 97%, 98%, 99%, or 100% identical to the amino acid sequences set forth in SEQ ID NO:45; (vii) a VH that is at least 75%, 80%, 85%, 90%, 91%, 92%, 93%, 94%, 95%, 96%, 97%, 98%, 99%, or 100% identical to the amino acid sequences set forth in SEQ ID NO:49, and a VL that is at least 75%, 80%, 85%, 90%, 91%, 92%, 93%, 94%, 95%, 96%, 97%, 98%, 99%, or 100% identical to the amino acid sequences set forth in SEQ ID NO:50; (viii) a VH that is at least 75%, 80%, 85%, 90%, 91%, 92%, 93%, 94%, 95%, 96%, 97%, 98%, 99%, or 100% identical to the amino acid sequences set forth in SEQ ID NO:57, and a VL that is at least 75%, 80%, 85%, 90%, 91%, 92%, 93%, 94%, 95%, 96%, 97%, 98%, 99%, or 100% identical to the amino acid sequences set forth in SEQ ID NO:58; (ix) a VH that is at least 75%, 80%, 85%, 90%, 91%, 92%, 93%, 94%, 95%, 96%, 97%, 98%, 99%, or 100% identical to the amino acid sequences set forth in SEQ ID NO:61, and a VL that is at least 75%, 80%, 85%, 90%, 91%, 92%, 93%, 94%, 95%, 96%, 97%, 98%, 99%, or 100% identical to the amino acid sequences set forth in SEQ ID NO:58; (x) a VH that is at least 75%, 80%, 85%, 90%, 91%, 92%, 93%, 94%, 95%, 96%, 97%, 98%, 99%, or 100% identical to the amino acid sequences set forth in SEQ ID NO:64, and a VL that is at least 75%, 80%, 85%, 90%, 91%, 92%, 93%, 94%, 95%, 96%, 97%, 98%, 99%, or 100% identical, or is identical to the amino acid sequences set forth in SEQ ID NO:58.

In some embodiments of the above aspect, the anti-αvβ1 antibody comprises (i) a VH comprising 20 or fewer (e.g. 19, 18, 17, 16, 15, 14, 13, 12, 11, 10, 9, 8, 7, 6, 5, 4, 3, 2, or 1) substitutions, insertions or deletions in the amino acid sequences set forth in SEQ ID NO:11, and a VL comprising 20 or fewer (e.g. 19, 18, 17, 16, 15, 14, 13, 12, 11, 10, 9, 8, 7, 6, 5, 4, 3, 2, or 1) substitutions, insertions or deletions in the amino acid sequences set forth in SEQ ID NO:12; (ii) a VH comprising 20 or fewer (e.g. 19, 18, 17, 16, 15, 14, 13, 12, 11, 10, 9, 8, 7, 6, 5, 4, 3, 2, or 1) substitutions, insertions or deletions in the amino acid sequences set forth in SEQ ID NO:21, and a VL comprising 20 or fewer (e.g. 19, 18, 17, 16, 15, 14, 13, 12, 11, 10, 9, 8, 7, 6, 5, 4, 3, 2, or 1) substitutions, insertions or deletions in the amino acid sequences set forth in SEQ ID NO:22; (iii) a VH comprising 20 or fewer (e.g. 19, 18, 17, 16, 15, 14, 13, 12, 11, 10, 9, 8, 7, 6, 5, 4, 3, 2, or 1) substitutions, insertions or deletions in the amino acid sequences set forth in SEQ ID NO:27, and a VL comprising 20 or fewer (e.g. 19, 18, 17, 16, 15, 14, 13, 12, 11, 10, 9, 8, 7, 6, 5, 4, 3, 2, or 1) substitutions, insertions or deletions in the amino acid sequences set forth in SEQ ID NO:28; (iv) a VH comprising 20 or fewer (e.g. 19, 18, 17, 16, 15, 14, 13, 12, 11, 10, 9, 8, 7, 6, 5, 4, 3, 2, or 1) substitutions, insertions or deletions in the amino acid sequences set forth in SEQ ID NO:30, and a VL comprising 20 or fewer (e.g. 19, 18, 17, 16, 15, 14, 13, 12, 11, 10, 9, 8, 7, 6, 5, 4, 3, 2, or 1) substitutions, insertions or deletions in the amino acid sequences set forth in SEQ ID NO:12; (v) a VH comprising 20 or fewer (e.g. 19, 18, 17, 16, 15, 14, 13, 12, 11, 10, 9, 8, 7, 6, 5, 4, 3, 2, or 1) substitutions, insertions or deletions in the amino acid sequences set forth in SEQ ID NO:35, and a VL comprising 20 or fewer (e.g. 19, 18, 17, 16, 15, 14, 13, 12, 11, 10, 9, 8, 7, 6, 5, 4, 3, 2, or 1) substitutions, insertions or deletions in the amino acid sequences set forth in SEQ ID NO:22; (vi) a VH comprising 20 or fewer (e.g. 19, 18, 17, 16, 15, 14, 13, 12, 11, 10, 9, 8, 7, 6, 5, 4, 3, 2, or 1) substitutions, insertions or deletions in the amino acid sequences set forth in SEQ ID NO:44, and a VL comprising 20 or fewer (e.g. 19, 18, 17, 16, 15, 14, 13, 12, 11, 10, 9, 8, 7, 6, 5, 4, 3, 2, or 1) substitutions, insertions or deletions in the amino acid sequences set forth in SEQ ID NO:45; (vii) a VH comprising 20 or fewer (e.g. 19, 18, 17, 16, 15, 14, 13, 12, 11, 10, 9, 8, 7, 6, 5, 4, 3, 2, or 1) substitutions, insertions or deletions in the amino acid sequences set forth in SEQ ID NO:49, and a VL comprising 20 or fewer (e.g. 19, 18, 17, 16, 15, 14, 13, 12, 11, 10, 9, 8, 7, 6, 5, 4, 3, 2, or 1) substitutions, insertions or deletions in the amino acid sequences set forth in SEQ ID NO:50; (viii) a VH comprising 20 or fewer (e.g. 19, 18, 17, 16, 15, 14, 13, 12, 11, 10, 9, 8, 7, 6, 5, 4, 3, 2, or 1) substitutions, insertions or deletions in the amino acid sequences set forth in SEQ ID NO:57, and a VL comprising 20 or fewer (e.g. 19, 18, 17, 16, 15, 14, 13, 12, 11, 10, 9, 8, 7, 6, 5, 4, 3, 2, or 1) substitutions, insertions or deletions in the amino acid sequences set forth in SEQ ID NO:58; (ix) a VH comprising 20 or fewer (e.g. 19, 18, 17, 16, 15, 14, 13, 12, 11, 10, 9, 8, 7, 6, 5, 4, 3, 2, or 1) substitutions, insertions or deletions in the amino acid sequences set forth in SEQ ID NO:61, and a VL comprising 20 or fewer (e.g. 19, 18, 17, 16, 15, 14, 13, 12, 11, 10, 9, 8, 7, 6, 5, 4, 3, 2, or 1) substitutions, insertions or deletions in the amino acid sequences set forth in SEQ ID NO:58; (x) a VH comprising 20 or fewer (e.g. 19, 18, 17, 16, 15, 14, 13, 12, 11, 10, 9, 8, 7, 6, 5, 4, 3, 2, or 1) substitutions, insertions or deletions in the amino acid sequences set forth in SEQ ID NO:64, and a VL comprising 20 or fewer (e.g. 19, 18, 17, 16, 15, 14, 13, 12, 11, 10, 9, 8, 7, 6, 5, 4, 3, 2, or 1) substitutions, insertions or deletions in the amino acid sequences set forth in SEQ ID NO:58.

In another aspect, this disclosure features an antibody that binds to both αvβ1 and αvβ6 integrins but not to other integrins (e.g., other RGD-family integrins), wherein the antibody comprises a VH comprising VHCDR1, VHCDR2, and VHCDR3, and a VL comprising VLCDR1, VLCDR2, and VLCDR3, wherein VHCDR1, VHCDR2, VHCDR3, VLCDR1, VLCDR2, and VLCDR3 comprise: (i) SEQ ID NOs:37, 39, 40, 65, 66, and 67, respectively; (ii) SEQ ID NOs: 37, 39, 40, 65, 66, and 69, respectively; (iii) SEQ ID NOs: 37, 39, 46, 18, 47, and 71, respectively; or (iv) SEQ ID NOs:37, 39, 73, 74, 42, and 75, respectively.

In some embodiments of the above aspect, the antibody that binds to both αvβ1 and αvβ6 integrins but not to other integrins comprises (i) a VH that is at least 75%, 80%, 85%, 90%, 91%, 92%, 93%, 94%, 95%, 96%, 97%, 98%, 99%, or 100% identical to the amino acid sequences set forth in SEQ ID NO:44, and a VL that is at least 75%, 80%, 85%, 90%, 91%, 92%, 93%, 94%, 95%, 96%, 97%, 98%, 99%, or 100% identical to the amino acid sequences set forth in SEQ ID NO:68; (ii) a VH that is at least 75%, 80%, 85%, 90%, 91%, 92%, 93%, 94%, 95%, 96%, 97%, 98%, 99%, or 100% identical to the amino acid sequences set forth in SEQ ID NO:44, and a VL that is at least 75%, 80%, 85%, 90%, 91%, 92%, 93%, 94%, 95%, 96%, 97%, 98%, 99%, or 100% identical to the amino acid sequences set forth in SEQ ID NO:70; (iii) a VH that is at least 75%, 80%, 85%, 90%, 91%, 92%, 93%, 94%, 95%, 96%, 97%, 98%, 99%, or 100% identical to the amino acid sequences set forth in SEQ ID NO:49, and a VL that is at least 75%, 80%, 85%, 90%, 91%, 92%, 93%, 94%, 95%, 96%, 97%, 98%, 99%, or 100% identical to the amino acid sequences set forth in SEQ ID NO:72; (iv) a VH that is at least 75%, 80%, 85%, 90%, 91%, 92%, 93%, 94%, 95%, 96%, 97%, 98%, 99%, or 100% identical to the amino acid sequences set forth in SEQ ID NO:76, and a VL that is at least 75%, 80%, 85%, 90%, 91%, 92%, 93%, 94%, 95%, 96%, 97%, 98%, 99%, or 100% identical to the amino acid sequences set forth in SEQ ID NO:77.

In some embodiments of the above aspect, the antibody that binds to both αvβ1 and αvβ6 integrins but not to other integrins comprises (i) a VH comprising 20 or fewer (e.g. 19, 18, 17, 16, 15, 14, 13, 12, 11, 10, 9, 8, 7, 6, 5, 4, 3, 2, or 1) substitutions, insertions or deletions in the amino acid sequences set forth in SEQ ID NO:44, and a VL comprising 20 or fewer (e.g. 19, 18, 17, 16, 15, 14, 13, 12, 11, 10, 9, 8, 7, 6, 5, 4, 3, 2, or 1) substitutions, insertions or deletions in the amino acid sequences set forth in SEQ ID NO:68; (ii) a VH comprising 20 or fewer (e.g. 19, 18, 17, 16, 15, 14, 13, 12, 11, 10, 9, 8, 7, 6, 5, 4, 3, 2, or 1) substitutions, insertions or deletions in the amino acid sequences set forth in SEQ ID NO:44, and a VL comprising 20 or fewer (e.g. 19, 18, 17, 16, 15, 14, 13, 12, 11, 10, 9, 8, 7, 6, 5, 4, 3, 2, or 1) substitutions, insertions or deletions in the amino acid sequences set forth in SEQ ID NO:70; (iii) a VH comprising 20 or fewer (e.g. 19, 18, 17, 16, 15, 14, 13, 12, 11, 10, 9, 8, 7, 6, 5, 4, 3, 2, or 1) substitutions, insertions or deletions in the amino acid sequences set forth in SEQ ID NO:49, and a VL comprising 20 or fewer (e.g. 19, 18, 17, 16, 15, 14, 13, 12, 11, 10, 9, 8, 7, 6, 5, 4, 3, 2, or 1) substitutions, insertions or deletions in the amino acid sequences set forth in SEQ ID NO:72; (iv) a VH comprising 20 or fewer (e.g. 19, 18, 17, 16, 15, 14, 13, 12, 11, 10, 9, 8, 7, 6, 5, 4, 3, 2, or 1) substitutions, insertions or deletions in the amino acid sequences set forth in SEQ ID NO:76, and a VL comprising 20 or fewer (e.g. 19, 18, 17, 16, 15, 14, 13, 12, 11, 10, 9, 8, 7, 6, 5, 4, 3, 2, or 1) substitutions, insertions or deletions in the amino acid sequences set forth in SEQ ID NO:77.

In another aspect, this disclosure features an antibody that binds to αvβ1 and one or more integrins selected from the group consisting of αvβ6, αvβ3, αvβ5, αvβ8, α5β1, α8β1, and αIIBβ3, wherein the antibody comprises a VH comprising VHCDR1, VHCDR2, and VHCDR3, and a VL comprising VLCDR1, VLCDR2, and VLCDR3, wherein VHCDR1, VHCDR2, VHCDR3, VLCDR1, VLCDR2, and VLCDR3 comprise: (i) SEQ ID NOs:4, 6, 78, 79, 80, and 81, respectively; (ii) SEQ ID NOs: 85, 87, 88, 89, 90, and 91, respectively; (iii) SEQ ID NOs: 85, 87, 88, 89, 90, and 94, respectively; (iv) SEQ ID NOs:97, 99, 23, 24, 25, and 26, respectively; (v) SEQ ID NOs:14, 16, 17, 101, 102, and 103, respectively; or (vi) SEQ ID NOs:37, 39, 46, 105, 80, and 106, respectively.

In some embodiments of the above aspect, the antibody that binds to αvβ1 and one or more integrins selected from the group consisting of αvβ6, αvβ3, αvβ5, αvβ8, α5β1, α8β1, and αIIBβ3 comprises (i) a VH that is at least 75%, 80%, 85%, 90%, 91%, 92%, 93%, 94%, 95%, 96%, 97%, 98%, 99%, or 100% identical to the amino acid sequences set forth in SEQ ID NO:82, and a VL that is at least 75%, 80%, 85%, 90%, 91%, 92%, 93%, 94%, 95%, 96%, 97%, 98%, 99%, or 100% identical to the amino acid sequences set forth in SEQ ID NO:83; (ii) a VH that is at least 75%, 80%, 85%, 90%, 91%, 92%, 93%, 94%, 95%, 96%, 97%, 98%, 99%, or 100% identical to the amino acid sequences set forth in SEQ ID NO:92, and a VL that is at least 75%, 80%, 85%, 90%, 91%, 92%, 93%, 94%, 95%, 96%, 97%, 98%, 99%, or 100% identical to the amino acid sequences set forth in SEQ ID NO:93; (iii) a VH that is at least 75%, 80%, 85%, 90%, 91%, 92%, 93%, 94%, 95%, 96%, 97%, 98%, 99%, or 100% identical to the amino acid sequences set forth in SEQ ID NO:92, and a VL that is at least 75%, 80%, 85%, 90%, 91%, 92%, 93%, 94%, 95%, 96%, 97%, 98%, 99%, or 100% identical to the amino acid sequences set forth in SEQ ID NO:95; (iv) a VH that is at least 75%, 80%, 85%, 90%, 91%, 92%, 93%, 94%, 95%, 96%, 97%, 98%, 99%, or 100% identical to the amino acid sequences set forth in SEQ ID NO:100, and a VL that is at least 75%, 80%, 85%, 90%, 91%, 92%, 93%, 94%, 95%, 96%, 97%, 98%, 99%, or 100% identical to the amino acid sequences set forth in SEQ ID NO:28; (v) a VH that is at least 75%, 80%, 85%, 90%, 91%, 92%, 93%, 94%, 95%, 96%, 97%, 98%, 99%, or 100% identical to the amino acid sequences set forth in SEQ ID NO:21, and a VL that is at least 75%, 80%, 85%, 90%, 91%, 92%, 93%, 94%, 95%, 96%, 97%, 98%, 99%, or 100% identical to the amino acid sequences set forth in SEQ ID NO:104; (vi) a VH that is at least 75%, 80%, 85%, 90%, 91%, 92%, 93%, 94%, 95%, 96%, 97%, 98%, 99%, or 100% identical to the amino acid sequences set forth in SEQ ID NO:49, and a VL that is at least 75%, 80%, 85%, 90%, 91%, 92%, 93%, 94%, 95%, 96%, 97%, 98%, 99%, or 100% identical to the amino acid sequences set forth in SEQ ID NO:107.

In some embodiments of the above aspect, the antibody that binds to αvβ1 and one or more integrins selected from the group consisting of αvβ6, αvβ3, αvβ5, αvβ8, α5β1, α8β1, and αIIBβ3 comprises (i) a VH comprising 20 or fewer (e.g. 19, 18, 17, 16, 15, 14, 13, 12, 11, 10, 9, 8, 7, 6, 5, 4, 3, 2, or 1) substitutions, insertions or deletions in the amino acid sequences set forth in SEQ ID NO:82, and a VL comprising 20 or fewer (e.g. 19, 18, 17, 16, 15, 14, 13, 12, 11, 10, 9, 8, 7, 6, 5, 4, 3, 2, or 1) substitutions, insertions or deletions in the amino acid sequences set forth in SEQ ID NO:83; (ii) a VH comprising 20 or fewer (e.g. 19, 18, 17, 16, 15, 14, 13, 12, 11, 10, 9, 8, 7, 6, 5, 4, 3, 2, or 1) substitutions, insertions or deletions in the amino acid sequences set forth in SEQ ID NO:92, and a VL comprising 20 or fewer (e.g. 19, 18, 17, 16, 15, 14, 13, 12, 11, 10, 9, 8, 7, 6, 5, 4, 3, 2, or 1) substitutions, insertions or deletions in the amino acid sequences set forth in SEQ ID NO:93; (iii) a VH comprising 20 or fewer (e.g. 19, 18, 17, 16, 15, 14, 13, 12, 11, 10, 9, 8, 7, 6, 5, 4, 3, 2, or 1) substitutions, insertions or deletions in the amino acid sequences set forth in SEQ ID NO:92, and a VL comprising 20 or fewer (e.g. 19, 18, 17, 16, 15, 14, 13, 12, 11, 10, 9, 8, 7, 6, 5, 4, 3, 2, or 1) substitutions, insertions or deletions in the amino acid sequences set forth in SEQ ID NO:95; (iv) a VH comprising 20 or fewer (e.g. 19, 18, 17, 16, 15, 14, 13, 12, 11, 10, 9, 8, 7, 6, 5, 4, 3, 2, or 1) substitutions, insertions or deletions in the amino acid sequences set forth in SEQ ID NO:100, and a VL comprising 20 or fewer (e.g. 19, 18, 17, 16, 15, 14, 13, 12, 11, 10, 9, 8, 7, 6, 5, 4, 3, 2, or 1) substitutions, insertions or deletions in the amino acid sequences set forth in SEQ ID NO:28; (v) a VH comprising 20 or fewer (e.g. 19, 18, 17, 16, 15, 14, 13, 12, 11, 10, 9, 8, 7, 6, 5, 4, 3, 2, or 1) substitutions, insertions or deletions in the amino acid sequences set forth in SEQ ID NO:21, and a VL comprising 20 or fewer (e.g. 19, 18, 17, 16, 15, 14, 13, 12, 11, 10, 9, 8, 7, 6, 5, 4, 3, 2, or 1) substitutions, insertions or deletions in the amino acid sequences set forth in SEQ ID NO:104; (vi) a VH comprising 20 or fewer (e.g. 19, 18, 17, 16, 15, 14, 13, 12, 11, 10, 9, 8, 7, 6, 5, 4, 3, 2, or 1) substitutions, insertions or deletions in the amino acid sequences set forth in SEQ ID NO:49, and a VL comprising 20 or fewer (e.g. 19, 18, 17, 16, 15, 14, 13, 12, 11, 10, 9, 8, 7, 6, 5, 4, 3, 2, or 1) substitutions, insertions or deletions in the amino acid sequences set forth in SEQ ID NO:107.

In some embodiments of the above aspects, the antibody comprises a human IgG1, IgG2, IgG3, or IgG4 heavy chain constant region. In some embodiments of the above aspects, the antibody is modified to reduce or eliminate effector function. In some embodiments of the above aspects, the antibody comprises an aglycosylated human constant region. In some embodiments of the above aspects, the antibody comprises a hIgG1agly Fc, a hIgG2 SAA Fc, a hIgG4(S228P) Fc, or a hIgG4(S228P)/G1 agly Fc. In some embodiments of the above aspects, the antibody comprises a human kappa or human lambda light chain constant region. In some embodiments of the above aspects, the antibody is a whole antibody, a single domain antibody, a humanized antibody, a chimeric antibody, a bispecific antibody, a Fv, a scFv, a scFv-Fc, a scFv-CH3, an sc(Fv)2, an sc(Fv)2-Fc, an sc(Fv)2-CH3, a diabody, a nanobody, an Fab, and a F(ab′)2. In some embodiments of the above aspects, the antibody further comprises a half-life extending moiety. In some embodiments of the above aspects, the antibody further comprises a detectable label (e.g., a fluorescent label). In some embodiments of the above aspects, the antibody further comprises a therapeutic agent. In some embodiments of the above aspects, the antibody further comprises a radiotherapeutic agent. In some embodiments of the above aspects, the antibody further comprises a chemotherapeutic agent

In another aspect, provided herein is a pharmaceutical composition comprising the antibody of any one of the above aspects. In another aspect, provided herein is a polynucleotide or polynucleotides encoding the antibody of any one of the above aspects. The polynucleotide may encode an antibody that binds to its RGD-family integrin (e.g., αvβ1, αvβ6, αvβ1+other RGD family integrins) and comprise a nucleic acid encoding the three VH CDRs or VH of any of Exemplary Antibodies 1 to 20. In other cases, the polynucleotide may encode an antibody that binds to its RGD-family integrin (e.g., αvβ1, αvβ6, αvβ1+other RGD family integrins) and comprise a nucleic acid encoding the three VL CDRs or VL of any of Exemplary Antibodies 1 to 20. In some instances, the polynucleotide may encode an antibody that binds to its RGD-family integrin (e.g., αvβ1, αvβ6, αvβ1+other RGD family integrins) and comprise a nucleic acid encoding the three VH CDRs and three VL CDRs of any of Exemplary Antibodies 1 to 20. In yet other instances, the polynucleotide may encode an antibody that binds to its RGD-family integrin (e.g., αvβ1, αvβ6, αvβ1+other RGD family integrins) and comprise a nucleic acid encoding the VH and VL of any of Exemplary Antibodies 1 to 20. In another aspect, provided herein is a vector or vectors (e.g., expression vector(s)) comprising the polynucleotide or polynucleotides of the above aspect. In a further aspect, provided herein is a host cell comprising the polynucleotide or polynucleotides of the above aspect, or the vector or vectors (e.g., expression vector(s)) of the above aspect.

In another aspect, provided herein is a method of making an anti-integrin antibody, the method comprising: (a) culturing the host cell under conditions that permit expression of the antibody; and (b) isolating the antibody. In some embodiments of the above aspect, the method further comprises formulating the antibody as a sterile formulation suitable for administration to a human.

In another aspect, provided herein is a method of treating or preventing fibrosis in a human subject in need thereof, the method comprising administering to the human subject a therapeutically effective amount of an anti-integrin antibody described herein (e.g. Exemplary Antibodies 1-20). In some embodiments of the above aspect, the fibrosis is selected from the group consisting of liver fibrosis, lung fibrosis, kidney fibrosis, cardiac fibrosis, arthrofibrosis, mediastinal fibrosis, myelofibrosis, nephrogenic systemic fibrosis, Peyronie's disease, progressive massive fibrosis, small airway fibrosis, fibrosis associated with chronic obstructive pulmonary disease, and retroperitoneal fibrosis. In some embodiments, the fibrosis is liver fibrosis. In some embodiments, the fibrosis is idiopathic pulmonary fibrosis. In some embodiments, the fibrosis is scleroderma/systemic sclerosis.

In another aspect, provided herein is a method of treating or preventing cancer in a human subject in need thereof, the method comprising administering to the human subject a therapeutically effective amount of an anti-integrin antibody described herein (e.g. Exemplary Antibodies 1-20). In some embodiments of the above aspect, the cancer is of epithelial origin, and optionally wherein the cancer of epithelial origin is a squamous cell carcinoma, an adenocarcinoma, a transitional cell carcinoma, or a basal cell carcinoma. In some embodiments, the cancer is selected from the group consisting of pancreatic cancer, breast cancer, melanoma, prostate cancer, ovarian cancer, cervical cancer, brain and central nervous system tumors, and glioblastoma.

In another aspect, provided herein is a method of inhibiting platelet aggregation in a human subject in need thereof, the method comprising administering to the human subject a therapeutically effective amount of an anti-integrin antibody described herein (e.g. Exemplary Antibodies 1-20). In some embodiments of the above aspect, the inhibition is for treatment of acute coronary syndrome.

In another aspect, provided herein is a method of treating or preventing an ophthalmology disease or disorder in a human subject in need thereof, the method comprising administering to the human subject a therapeutically effective amount of an anti-integrin antibody described herein (e.g. Exemplary Antibodies 1-20). In some embodiments of the above aspect, the ophthalmology disease or disorder is selected from the group consisting of age-related macular degeneration (AMD), wet AMD, macular edema, and diabetic retinopathy.

In another aspect, provided herein is a method of treating or preventing acute kidney injury, acute lung injury, or acute liver injury in a human subject in need thereof, the method comprising administering to the human subject a therapeutically effective amount of an anti-integrin antibody described herein (e.g. Exemplary Antibodies 1-20).

In another aspect, provided herein is a method of treating or preventing Nonalcoholic fatty liver disease (NAFLD) in a human subject in need thereof, the method comprising administering to the human subject a therapeutically effective amount of the antibody of an anti-integrin antibody described herein (e.g. any one or more of Exemplary Antibodies 1-20). In some embodiments, the NAFLD is nonalcoholic steatohepatitis (NASH).

In another aspect, provided herein is a method of identifying an antibody that specifically binds to αvβ1 integrin but not to other integrins from a population of antibodies, the method comprising selecting the antibody using guided selection with a guide antibody that is any anti-integrin antibody described herein (e.g. Exemplary Antibodies 1-20). In some embodiments, the guide antibody comprises the six CDRs of any one of Exemplary antibody 5, 11, and 12. In some embodiments, the guide antibody comprises the VH and/or VL of any one of Exemplary antibody 5, 11, and 12. In some embodiments, the population of antibodies comprises an antibody library expressed on the surface of prokaryotic cells. In some embodiments, the population of antibodies comprises an antibody library expressed on the surface of eukaryotic cells. In some embodiments, the population of antibodies comprises an antibody library expressed on the surface of yeast cells. In some embodiments, the method comprises a step of selecting an antibody that binds to a polypeptide or polypeptides comprising the extracellular domains of αv and β1, optionally wherein the step is performed in the absence of cations, in the presence of calcium and magnesium, or in the presence of manganese, and also optionally, where the selection is performed by MACS and/or FACS. In some embodiments, the methods further comprise depleting antibodies that bind to one or more integrins selected from the group consisting of αvβ3, αvβ5, αvβ6, αvβ8, α5β1, α8β1, and α4β1. In some embodiments, the methods further comprise enriching for antibodies that specifically bind to αvβ1 integrin by selecting for antibodies that bind to αvβ1 integrin. The selections can be done in one or more rounds (e.g., one, two, three, four, or five rounds). In some embodiments, the methods further comprise affinity maturing the selected antibodies.

In a further aspect, provided herein is a method of identifying an antibody from a population of antibodies, wherein the antibody specifically binds to both αvβ1 and αvβ6 integrins, the method comprising selecting the antibody using guided selection with a guide antibody that is anti-integrin antibody described herein (e.g. any one or more of Exemplary Antibodies 1-20). In some embodiments, the guide antibody comprises the six CDRs of any one of Exemplary antibody 5, 11, and 12. In some embodiments, the guide antibody comprises the VH and/or VL of any one of Exemplary antibody 5, 11, and 12. In some embodiments, the population of antibodies comprises an antibody library expressed on the surface of prokaryotic cells. In some embodiments, the population of antibodies comprises an antibody library expressed on the surface of eukaryotic cells. In some embodiments, the population of antibodies comprises an antibody library expressed on the surface of yeast cells. In some embodiments, the method comprises a step of selecting an antibody that binds to a polypeptide or polypeptides comprising the extracellular domains of αv and β1 and/or the extracellular domains of αv and β6, optionally wherein the step is performed in the absence of cations, in the presence of calcium and magnesium, or in the presence of manganese, and also optionally, wherein the selection is performed by MACS and/or FACS. In some embodiments, the method further comprises depleting antibodies that bind to one or more integrins selected from the group consisting of αvβ3, αvβ5, αvβ8, α5β1, α8β1, and α4β1. In some embodiments, the method further comprises enriching for antibodies that specifically bind to αvβ1 and αvβ6 integrin by selecting for antibodies that bind to αvβ1 and αvβ6 integrins. The selection can be done in one or more rounds (e.g., one, two, three, four, or five rounds). In some embodiments, the method further comprises affinity maturing the selected antibodies.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, the exemplary methods and materials are described below. All publications, patent applications, patents, and other references mentioned herein are incorporated by reference in their entirety. In case of conflict, the present application, including definitions, will control. The materials, methods, and examples are illustrative only and not intended to be limiting.

Other features and advantages of the invention will be apparent from the following detailed description and from the claims.

### DESCRIPTION OF DRAWINGS

FIG. 1 depicts a general outline for the triage of αvβ1 specific, αvβ1/αvβ6 specific, and αvβ1 plus one or more integrins specific antibodies.

FIGS. 2A-2K show examples of observed binding kinetics for αvβ1 specific, non-specific, and partially selective antibodies.

FIGS. 3A-3E depict examples of observed binding kinetics for αvβ1 specific, non-specific, and partially selective antibodies.

FIGS. 4A-4J show monovalent binding affinity for recombinant αvβ1.

FIGS. 5A-5E show examples of observed binding titrations for αvβ1 specific and partially selective antibodies.

FIGS. 6A-6J show examples of observed binding titrations for αvβ1 specific and partially selective antibodies.

FIGS. 7A-7E depict examples of αvβ1 LAP adhesion inhibition.

FIG. 8 shows examples of α4β1 VCAM adhesion inhibition

FIGS. 9A-9D provide examples of observed binding to MRC9 (human fibroblast cells) and BLO-11 (murine fibroblast cells).

FIGS. 10A-10C illustrate examples of PAI-1 inhibition.

### DETAILED DESCRIPTION

This disclosure features antibodies that bind to integrins such as the RGD-binding integrins. Provided are antibodies that specifically bind the αvβ1 integrin. In some instances, provided herein are antibodies that bind to αvβ1 integrin but not to other integrins (e.g., these antibodies do not bind to other RGD-binding integrins, or other αv- or β1-containing integrin heterodimers). Also provided herein are antibodies that bind to both αvβ1 and αvβ6 integrin but not to other integrins (e.g. these antibodies do not bind to other RGD-binding integrins, or other αv-, β1-, or β6-containing integrin heterodimers). In some instances, provided herein are antibodies that bind to αvβ1 and one or more integrins selected from the group consisting of αvβ3, αvβ5, αvβ6, αvβ8, α5β1, α8β1, and αIIBβ3. The antibodies described herein are useful in the treatment or prevention of disorders such as any fibrotic disease or conditions, cancer (e.g. epithelial cancer), and ophthalmic diseases.

### Integrins

The αv integrins (αvβ1, αvβ3, αvβ5, αvβ6, and αvβ8) have the capability to bind and activate the pro-fibrotic cytokine transforming growth factor-β (TGFβ) and have been implicated in various fibrotic diseases and cancers. Blocking αv integrins can potentially reduce the downstream effects of TGFβ signaling. αvβ1 integrin is highly expressed on activated fibroblasts, directly binds to the latency-associated peptide (LAP) of TGFβ1 and mediates TGFβ1 activation, making it desirable to generate antibodies against the αvβ1 integrin. However, due to the fact that αv and (31 subunits are individually present in numerous integrin dimer pairs, it has been extremely challenging to generate heterodimer-specific antibodies against the αvβ1 integrin (see, e.g., Reed et al., Science Translational Medicine, 7(288):288ra79 (2015); Wilkinson et al., Eur. J Pharmacol., 842(2019) 239-247). In fact, no such anti-αvβ1 integrin specific antibodies have been described in the art.

The αvβ6 integrin is a member of the RGD-binding integrins. While the αᵥ subunit can form a heterodimer with a variety of β subunits (β1, β3, β5, β6 and β8), the β6 subunit can only be expressed as a heterodimer with the αv subunit. The extracellular and cytoplasmic domains of the β6 subunit mediate different cellular activities: the extracellular and transmembrane domains have been shown to mediate TGF-β activation and adhesion; whereas the cytoplasmic domain of the β6 subunit contains a unique 11-amino acid sequence that is important in mediating αvβ6 regulated cell proliferation, MMP production, migration, and promotes survival.

Integrin αv subunit is also known as ITGAV, CD51, MSK8, VNRA, VTNR, vitronectin receptor, or integrin subunit alpha V. The amino acid sequence of the human integrin αv protein (Uniprot Accession No. P06756-1) is shown below.

(SEQ ID NO: 1)
MAFPPRRRLRLGPRGLPLLLSGLLLPLCRAFNLDVDSPAEYSGPEGSYFG
FAVDFFVPSASSRMFLLVGAPKANTTQPGIVEGGQVLKCDWSSTRRCQPI
EFDATGNRDYAKDDPLEFKSHQWFGASVRSKQDKILACAPLYHWRTEMKQ
EREPVGTCFLQDGTKTVEYAPCRSQDIDADGQGFCQGGFSIDFTKADRVL
LGGPGSFYWQGQLISDQVAEIVSKYDPNVYSIKYNNQLATRTAQAIFDDS
YLGYSVAVGDFNGDGIDDFVSGVPRAARTLGMVYIYDGKNMSSLYNFTGE
QMAAYFGFSVAATDINGDDYADVFIGAPLFMDRGSDGKLQEVGQVSVSLQ
RASGDFQTTKLNGFEVFARFGSAIAPLGDLDQDGFNDIAIAAPYGGEDKK
GIVYIFNGRSTGLNAVPSQILEGQWAARSMPPSFGYSMKGATDIDKNGYP
DLIVGAFGVDRAILYRARPVITVNAGLEVYPSILNQDNKTCSLPGTALKV
SCFNVRFCLKADGKGVLPRKLNFQVELLLDKLKQKGAIRRALFLYSRSPS
HSKNMTISRGGLMQCEELIAYLRDESEFRDKLTPITIFMEYRLDYRTAAD
TTGLQPILNQFTPANISRQAHILLDCGEDNVCKPKLEVSVDSDQKKIYIG
DDNPLTLIVKAQNQGEGAYEAELIVSIPLQADFIGVVRNNEALARLSCAF
KTENQTRQVVCDLGNPMKAGTQLLAGLRFSVHQQSEMDTSVKFDLQIQSS
NLFDKVSPVVSHKVDLAVLAAVEIRGVSSPDHVFLPIPNWEHKENPETEE
DVGPVVQHIYELRNNGPSSFSKAMLHLQWPYKYNNNTLLYILHYDIDGPM
NCTSDMEINPLRIKISSLQTTEKNDTVAGQGERDHLITKRDLALSEGDIH
TLGCGVAQCLKIVCQVGRLDRGKSAILYVKSLLWTETFMNKENQNHSYSL
KSSASFNVIEFPYKNLPIEDITNSTLVTTNVTWGIQPAPMPVPVWVIILA
VLAGLLLLAVLVFVMYRMGFFKRVRPPQEEQEREQLQPHENGEGNSET

Integrin β1 subunit is also known as ITGB1, CD29, FNRB, GPIIA, MDF2, MSK12, VLA-BETA, VLAB, or integrin subunit beta 1. The amino acid sequence of the human integrin β1 protein (Uniprot Accession No. P05556-1) is shown below.

(SEQ ID NO: 2)
MNLQPIFWIGLISSVCCVFAQTDENRCLKANAKSCGECIQAGPNCGWCTN
STFLQEGMPTSARCDDLEALKKKGCPPDDIENPRGSKDIKKNKNVTNRSK
GTAEKLKPEDITQIQPQQLVLRLRSGEPQTFTLKFKRAEDYPIDLYYLMD
LSYSMKDDLENVKSLGTDLMNEMRRITSDFRIGFGSFVEKTVMPYISTTP
AKLRNPCTSEQNCTSPFSYKNVLSLTNKGEVFNELVGKQRISGNLDSPEG
GFDAIMQVAVCGSLIGWRNVTRLLVFSTDAGFHFAGDGKLGGIVLPNDGQ
CHLENNMYTMSHYYDYPSIAHLVQKLSENNIQTIFAVTEEFQPVYKELKN
LIPKSAVGTLSANSSNVIQLIIDAYNSLSSEVILENGKLSEGVTISYKSY
CKNGVNGTGENGRKCSNISIGDEVQFEISITSNKCPKKDSDSFKIRPLGF
TEEVEVILQYICECECQSEGIPESPKCHEGNGTFECGACRCNEGRVGRHC
ECSTDEVNSEDMDAYCRKENSSEICSNNGECVCGQCVCRKRDNTNEIYSG
KFCECDNFNCDRSNGLICGGNGVCKCRVCECNPNYTGSACDCSLDTSTCE
ASNGQICNGRGICECGVCKCTDPKFQGQTCEMCQTCLGVCAEHKECVQCR
AFNKGEKKDTCTQECSYFNITKVESRDKLPQPVQPDPVSHCKEKDVDDCW
FYFTYSVNGNNEVMVHVVENPECPTGPDIIPIVAGVVAGIVLIGLALLLI
WKLLMIIHDRREFAKFEKEKMNAKWDTGENPIYKSAVTTVVNPKYEGK

Integrin β6 subunit is also known as ITGB6, Al1H, or integrin subunit beta 6. The amino acid sequence of the human integrin β6 protein (Genbank® Accession No. NP_000879.2) is shown below.

| (SEQ ID NO: 116) | |
|---|---|
| 1 | mgiellclff lflgrndhvQ ggcalggaet cedclligpq cawcaQenft hpsgvgercd |
| 61 | tpanllakgc qlnfienpvs qveilknkpl svgrqknssd ivqiapqsli lklrpggaqt |
| 121 | lqvhvrqted ypvdlyylmd lsasmdddln tikelgsrls kemskltsnf rlgfgsfvek |
| 181 | pvspfvkttp eeianpcssi pyfclptfgf khilpltnda erfneivknq kisanidtpe |
| 241 | ggfdaimqaa vckekigwrn dslhllvfvs dadshfgmds klagivipnd glchldskne |
| 301 | ysmstvleyp tigqlidklv qnnvllifav tqeqvhlyen yaklipgatv gllqkdsgni |
| 361 | lqliisayee lrsevelevl gdteglnlsf taicnngtlf qhqkkcshmk vgdtasfsvt |
| 421 | vniphcerrs rhiiikpvgl gdalellvsp ecncdcqkev evnsskchhg ngsfqcgvca |
| 481 | chpghmgprc ecgedmlstd sckeapdhps csgrgdcycg qcichlspyg niygpycqcd |
| 541 | nfscvrhkgl lcggngdcdc gecvcrsgwt geycncttst dscvsedgvl csgrgdcvcg |
| 601 | kcvctnpgas gptcercptc gdpcnskrsc iechlsaagq areecvdkck lagatiseee |
| 661 | dfskdgsysc slqgenecli tflittdneg ktiihsinek dcpkppnipm imlgvslail |
| 721 | ligvvllciw kllvsfhdrk evakfeaers kakwqtgtnp lyrgststfk nvtykhrekq |
| 781 | kvdlstdc |

An exemplary amino acid sequence of the human integrin 33 protein can be found at Genbank® Accession No. NP_000203.2. An exemplary amino acid sequence of the human integrin β5 protein can be found at Genbank® Accession No. NP_002204.2. An exemplary amino acid sequence of the human integrin 38 protein can be found at Genbank® Accession No. NP_002205.1. An exemplary amino acid sequence of the human integrin α5 protein can be found at Genbank® Accession No. NP_002196.4. An exemplary amino acid sequence of the human integrin α8 protein can be found at Genbank® Accession No. NP_001278423.1. An exemplary amino acid sequence of the human integrin αIIB protein can be found at Genbank® Accession No. NP_000410.2.

The extracellular region of the integrin subunit alpha V corresponds to amino acids 31-993 of SEQ ID NO: 1. The extracellular region of the integrin subunit beta 1 corresponds to amino acids 21-728 of SEQ ID NO: 2.

### Anti-Integrin Antibodies

All of the anti-integrin antibodies of this disclosure bind to αvβ1. In some instances, the antibodies are specific for αvβ1 and do not bind other integrins. In some instances, the antibodies also bind αvβ6 but not other integrins. In yet other instances, the antibodies bind to one or more RGD-binding integrins selected from the group consisting of αvβ3, αvβ5, αvβ6, αvβ8, α5β1, α8β1, and αIIBβ3.

In some instances, the antibodies of this disclosure block the interaction of the integrin that the antibody binds to with its ligand. For example, the anti-integrin antibody blocks αvβ1 interaction with its ligand (e.g., LAP and fibronectin). In certain cases, the anti-integrin antibody blocks αvβ6 interaction with its ligand (e.g., LAP and fibronectin). In some instances, the antibodies of this disclosure are cation-dependent for binding its target (e.g., calcium and magnesium; or manganese). Examples of such antibodies are Exemplary Antibodies 1, 2, 4-14, 16, 17, 19, and 20. In some instances, the antibodies of this disclosure are not cation-dependent for binding its target. Examples of such antibodies are Exemplary Antibodies 3, 15, and 18.

In some instances, the antibodies of this disclosure bind its target integrin (e.g., αvβ1, αvβ6) on fibroblasts. Fibroblasts are the cell type responsible for extracellular matrix deposition in fibrotic diseases.

In some instances, the antibodies of this disclosure inhibit fibroblast TGFβ response.

In some instances, one or more of the antibodies of this disclosure are internalized. Examples of such antibodies are Exemplary Antibodies 4, 5, 17, and 19. Antibodies that are internalized can be used to deliver an agent that needs to be delivered into a cell (e.g. a small molecule or an intrabody).

In some instances, one or more of the antibodies of this disclosure bind to cynomolgus monkey, mouse, or rat αvβ1. Examples of such antibodies are Exemplary Antibodies 4, 5, 17, and 19.

In certain instances, this disclosure features an antibody that specifically binds to human αvβ1 and has one or more (e.g., 1, 2, 3, 4 or 5) of the following properties: (i) binds with high affinity of KD≤20 nM (bivalent affinity) to human αvβ1; (ii) blocks αvβ1 interaction with its ligand (e.g., LAP and fibronectin); (iii) is cation-dependent (e.g., calcium and magnesium; or manganese) or cation-independent for binding to human αvβ1; (iv) binds to αvβ1 on fibroblasts; and (v) inhibits fibroblast TGFβ response (e.g., as assessed by a LPA-induced PAI-1 assay).

In certain instances, this disclosure features an antibody that specifically binds to both human αvβ1 and human αvβ6 and has one or more (e.g., 1, 2, 3, 4 or 5) of the following properties: (i) bind with high affinity of KD≤20 nM (bivalent affinity) to human αvβ1, and with affinity of 100 nM (bivalent affinity) to human αvβ6; (ii) blocks αvβ1 and/or αvβ6 interaction with its ligand (e.g., LAP and fibronectin); (iii) is cation-dependent (e.g., calcium and magnesium; or manganese) for binding to human αvβ1 and/or αvβ6; (iv) binds to αvβ1 on fibroblasts; and (v) inhibits fibroblast TGFβ response (e.g., as assessed by a LPA-induced PAI-1 assay).

In certain instances, this disclosure features an antibody that specifically binds to both human αvβ1 and one or more of the other RGD-binding integrins and has one or more (e.g., 1, 2, 3, 4 or 5) of the following properties: (i) which bind with high affinity of KD≤20 nM (bivalent affinity) to human αvβ1, and with affinity of 100 nM (bivalent affinity) to other RGD binding integrins; (ii) blocks αvβ1 and/or RGD family integrin interaction with its ligand (e.g., LAP and fibronectin); (iii) is cation-dependent (e.g., calcium and magnesium; or manganese) or cation-independent for binding to human αvβ1 and/or RGD binding integrins; (iv) binds to αvβ1 and/or RGD binding integrins on fibroblasts; and (v) inhibits fibroblast TGFβ response (e.g., as assessed by a LPA-induced PAI-1 assay).

Usage of the term “antibody” in this disclosure is meant to cover a whole antibody (as opposed to a minibody, nanobody or antibody fragment), a bispecific antibody, a tetravalent antibody, a multispecific antibody, a minibody, a nanobody, and antibody fragments. In some instances, the anti-integrin antibody of this disclosure is a whole antibody. In certain instances, the heavy chain constant region of the anti-integrin antibody is a human IgG1, human IgG2, human IgG3, or human IgG4 constant region. In certain instances, the light constant region is a human kappa constant region. In other instances, the light constant region is a human lambda constant region. In some instances, the antibodies of this disclosure are designed to have low effector functionality (e.g., by Fc modifications such as N297Q, T299A, etc. See, also, Wang, X., Mathieu, M. & Brezski, R. J. Protein Cell (2018) 9: 63. doi.org/10.1007/si3238-017-0473-8 (incorporated by reference herein)). In some cases, the Fc moiety of the antibody is a hIgG1 Fc, a hIgG2 Fc, a hIgG3 Fc, a hIgG4 Fc, a hIgG1agly Fc, a hIgG2 SAA Fc, a hIgG4(S228P) Fc, or a hIgG4(S228P)/G1 agly Fc (in this format—that minimizes effector function—the CH1 and CH2 domains are IgG4 with a ‘fixed’ hinge (S228P) and is aglycosylated. The CH3 domain is hIgG1, or a hIgG4(S228P) agly Fc). In one case, the antibody has one of the following three scaffolds with reduced effector function: hIgG1 agly (N297Q); hIgG2 SAA (see, Vafa et al. Methods, 65(1):114-26 (2014); and hIgG4P/G1 agly (see, US 2012/0100140 A1).

For ease of description, the anti-integrin antibodies featured herein are divided into three Groups, namely Groups I-III.

Group I antibodies are antibodies that bind to αvβ1 integrin but no other integrin (e.g., other αv- or β1-containing or RGD family integrins). Group II antibodies are those that bind to αvβ1 and αvβ6 integrins but no other integrins. Finally, Group III antibodies bind to αvβ1 and one or more integrins selected from the group consisting of αvβ3, αvβ5, αvβ6, αvβ8, α5β1, α8β1, and αIIBβ3. These three groups of antibodies are elaborated on below.

A. Group I (anti-αvβ1 Integrin Specific Antibodies)

Numerous publications have suggested the challenges associated with generating antibodies specific to the αvβ1 integrin. This is partly due to the fact that αv and β1 subunits are individually present in numerous integrin dimer pairs (Reed et al., Sci Transl Med., 7:288 (2015)). The present inventors have succeeded in generating such anti-αvβ1 integrin-specific antibodies.

Accordingly, this disclosure provides antibodies that specifically bind to αvβ1 integrin and that do not bind to other integrins. In some instances, the antibodies do not bind other αv- or β1-containing integrin heterodimers. In some instances, the anti-αvβ1 antibodies do not bind other RGD integrins (e.g., αvβ3, αvβ5, αvβ6, αvβ8, α5β1, α8β1, and αIIBβ3 integrins). These antibodies all bind human αvβ1 integrin. Such antibodies includes Exemplary Antibodies 1-10, which bind with high affinity of KD≤20 nM (bivalent affinity) to human αvβ1.

### Exemplary Antibody 1

Exemplary Antibody 1 specifically binds human αvβ1. The amino acid sequences of the complementarity determining regions (CDRs) and the mature heavy chain variable region and light chain variable regions of Exemplary Antibody 1 are provided below.

| Domain | Kabat | Enhanced Chothia/AbM |
|---|---|---|
| VH CDR1 | SYYMH | YTFTSYYMH |
| | (SEQ ID NO: 3) | (SEQ ID NO: 4) |
| VH CDR2 | IINPSGGSTSYAQKFQG | IINPSGGSTS |
| | (SEQ ID NO: 5) | (SEQ ID NO: 6) |
| VH CDR3 | QQRHRRDYDYYYGMDV | QQRHRRDYDYYYGMDV |
| | (SEQ ID NO: 7) | (SEQ ID NO: 7) |
| VL CDR1 | RASQSVSSDYLA | RASQSVSSDYLA |
| | (SEQ ID NO: 8) | (SEQ ID NO: 8) |
| VL CDR2 | GASRRAT | GASRRAT |
| | (SEQ ID NO: 9) | (SEQ ID NO: 9) |
| VL CDR3 | QQAYSLPPT | QQAYSLPPT |
| | (SEQ ID NO: 10) | (SEQ ID NO: 10) |

Heavy Chain Variable Region (VH):
QVQLVQSGAEVKKPGASVKVSCKASGYTFTSYYMHWVRQAPGQGLEWMGI
INPSGGSTSYAQKFQGRVTMTRDTSTSTVYMELSSLRSEDTAVYYCARQQ
RHRRDYDYYYGMDVWGQGTTVTVSS (SEQ ID NO: 11)
Light Chain Variable Region (VL):
EIVLTQSPGTLSLSPGERATLSCRASQSVSSDYLAWYQQKPGQAPRLLIY
GASRRATGIPDRFSGSGSGTDFTLTISRLEPEDFAVYYCQQAYSLPPTFG
GGTKVEIK (SEQ ID NO: 12)

In some instances, the anti-αvβ1 antibody comprises a VH comprising the three VH CDRs and a VL comprising the three VL CDRs of Exemplary Antibody 1. The six CDRs can be based on any definition known in the art such as, but not limited to, Kabat, Chothia, enhanced Chothia, contact, IMGT, or Honegger definitions. These CDRs can be determined, e.g., by using the AbYsis database (www.bioinf.org.uk/abysis/sequence_input/key_annotation/key_annotation.cgi).

In one instance, an anti-αvβ1 antibody of this disclosure comprises (i) a VH comprising a VHCDR1 comprising the amino acid sequence set forth in SEQ ID NO:4, a VHCDR2 comprising the amino acid sequence set forth in SEQ ID NO: 6, and a VHCDR3 comprising the amino acid sequence set forth in SEQ ID NO: 7; and (ii) a VL comprising a VLCDR1 comprising the amino acid sequence set forth in SEQ ID NO:8, a VLCDR2 comprising the amino acid sequence set forth in SEQ ID NO: 9, and a VLCDR3 comprising the amino acid sequence set forth in SEQ ID NO: 10. In another instance, an anti-αvβ1 antibody of this disclosure comprises (i) a VH comprising a VHCDR1 comprising the amino acid sequence set forth in SEQ ID NO:3, a VHCDR2 comprising the amino acid sequence set forth in SEQ ID NO: 5, and a VHCDR3 comprising the amino acid sequence set forth in SEQ ID NO: 7; and (ii) a VL comprising a VLCDR1 comprising the amino acid sequence set forth in SEQ ID NO:8, a VLCDR2 comprising the amino acid sequence set forth in SEQ ID NO:9, and a VLCDR3 comprising the amino acid sequence set forth in SEQ ID NO:10.

In some instances, the anti-αvβ1 antibody comprises a VH that is at least 75%, 80%, 85%, 90%, 91%, 92%, 93%, 94%, 95%, 96%, 97%, 98%, or 99% identical to the amino acid sequence set forth in SEQ ID NO:11. In some instances, the anti-αvβ1 antibody comprises a VL that is at least 75%, 80%, 85%, 90%, 91%, 92%, 93%, 94%, 95%, 96%, 97%, 98%, or 99% identical to the amino acid sequence set forth in SEQ ID NO:12. In one instance, the anti-αvβ1 antibody comprises a VH that is at least 85% identical to the amino acid sequence set forth in SEQ ID NO:11 and a VL that is at least 85% identical to the amino acid sequence set forth in SEQ ID NO:12. In another instance, the anti-αvβ1 antibody comprises a VH that is at least 90% identical to the amino acid sequence set forth in SEQ ID NO:11 and a VL that is at least 90% identical to the amino acid sequence set forth in SEQ ID NO: 12. In yet another instance, the anti-αvβ1 antibody comprises a VH that is identical to the amino acid sequence set forth in SEQ ID NO:11 and a VL that is identical to the amino acid sequence set forth in SEQ ID NO:12.

In certain instances, an antibody of this disclosure that binds to αvβ1 is one that competes with or binds to the same epitope as a reference antibody with a VH having the amino acid sequence set forth in SEQ ID NO:11 and a VL having the amino acid sequence set forth in SEQ ID NO: 12.

### Exemplary Antibody 2

Exemplary Antibody 2 specifically binds human αvβ1. The amino acid sequences of the CDRs and the mature heavy chain variable region and light chain variable regions of Exemplary Antibody 2 are provided below.

| Domain | Kabat | Enhanced Chothia/AbM |
|---|---|---|
| VH CDR1 | SYGMH | FTFSSYGMH |
| | (SEQ ID NO: 13) | (SEQ ID NO: 14) |
| VH CDR2 | VISYDGSNKYYADSVKG | VISYDGSNKY |
| | (SEQ ID NO: 15) | (SEQ ID NO: 16) |
| VH CDR3 | GGPTRGDGTRVYYYGMDV | GGPTRGDGTRVYYYGMDV |
| | (SEQ ID NO: 17) | (SEQ ID NO: 17) |
| VL CDR1 | RASQSVSSNLA | RASQSVSSNLA |
| | (SEQ ID NO: 18) | (SEQ ID NO: 18) |
| VL CDR2 | SASTRAT | SASTRAT |
| | (SEQ ID NO: 19) | (SEQ ID NO: 19) |
| VL CDR3 | QQYYHHPFT | QQYYHHPFT |
| | (SEQ ID NO: 20) | (SEQ ID NO: 20) |

VH
QVQLVESGGGVVQPGRSLRLSCAASGFTFSSYGMHWVRQAPGKGLEWVAV
ISYDGSNKYYADSVKGRFTISRDNSKNTLYLQMNSLRAEDTAVYYCARGG
PTRGDGTRVYYYGMDVWGQGTTVTVSS (SEQ ID NO: 21)
VL
EIVMTQSPATLSVSPGERATLSCRASQSVSSNLAWYQQKPGQAPRLLIYS
ASTRATGIPARFSGSGSGTEFTLTISSLQSEDFAVYYCQQYYHHPFTFGG
GTKVEIK (SEQ ID NO: 22)

In some instances, the anti-αvβ1 antibody comprises a VH comprising the three VH CDRs and a VL comprising the three VL CDRs of Exemplary Antibody 2. The six CDRs can be based on any definition known in the art such as, but not limited to, Kabat, Chothia, enhanced Chothia, contact, IMGT, or Honegger definitions. These CDRs can be determined, e.g., by using the AbYsis database.

In one instance, an anti-αvβ1 antibody of this disclosure comprises (i) a VH comprising a VHCDR1 comprising the amino acid sequence set forth in SEQ ID NO:14, a VHCDR2 comprising the amino acid sequence set forth in SEQ ID NO:16, and a VHCDR3 comprising the amino acid sequence set forth in SEQ ID NO: 17; and (ii) a VL comprising a VLCDR1 comprising the amino acid sequence set forth in SEQ ID NO:18, a VLCDR2 comprising the amino acid sequence set forth in SEQ ID NO: 19, and a VLCDR3 comprising the amino acid sequence set forth in SEQ ID NO:20. In another instance, an anti-αvβ1 antibody of this disclosure comprises (i) a VH comprising a VHCDR1 comprising the amino acid sequence set forth in SEQ ID NO:13, a VHCDR2 comprising the amino acid sequence set forth in SEQ ID NO:15, and a VHCDR3 comprising the amino acid sequence set forth in SEQ ID NO:17; and (ii) a VL comprising a VLCDR1 comprising the amino acid sequence set forth in SEQ ID NO:18, a VLCDR2 comprising the amino acid sequence set forth in SEQ ID NO:19, and a VLCDR3 comprising the amino acid sequence set forth in SEQ ID NO:20.

In some instances, the anti-αvβ1 antibody comprises a VH that is at least 75%, 80%, 85%, 90%, 91%, 92%, 93%, 94%, 95%, 96%, 97%, 98%, or 99% identical to the amino acid sequence set forth in SEQ ID NO:21. In some instances, the anti-αvβ1 antibody comprises a VL that is at least 75%, 80%, 85%, 90%, 91%, 92%, 93%, 94%, 95%, 96%, 97%, 98%, or 99% identical to the amino acid sequence set forth in SEQ ID NO:22. In one instance, the anti-αvβ1 antibody comprises a VH that is at least 85% identical to the amino acid sequence set forth in SEQ ID NO:21 and a VL that is at least 85% identical to the amino acid sequence set forth in SEQ ID NO:22. In another instance, the anti-αvβ1 antibody comprises a VH that is at least 90% identical to the amino acid sequence set forth in SEQ ID NO:21 and a VL that is at least 90% identical to the amino acid sequence set forth in SEQ ID NO:22. In yet another instance, the anti-αvβ1 antibody comprises a VH that is identical to the amino acid sequence set forth in SEQ ID NO:21 and a VL that is identical to the amino acid sequence set forth in SEQ ID NO:22.

In certain instances, an antibody of this disclosure that binds to αvβ1 is one that competes with or binds to the same epitope as a reference antibody with a VH having the amino acid sequence set forth in SEQ ID NO:21 and a VL having the amino acid sequence set forth in SEQ ID NO:22.

### Exemplary Antibody 3

Exemplary Antibody 3 specifically binds human αvβ1. This antibody shows cation-independent binding to its target. The amino acid sequences of the CDRs and the mature heavy chain variable region and light chain variable regions of Exemplary Antibody 3 are provided below.

| Domain | Kabat | Enhanced Chothia/AbM |
|---|---|---|
| VH CDR1 | SYYMH | YTFTSYYMH |
| | (SEQ ID NO: 3) | (SEQ ID NO: 4) |
| VH CDR2 | IINPSGGSTSYAQKFQG | IINPSGGSTS |
| | (SEQ ID NO: 5) | (SEQ ID NO: 6) |
| VH CDR3 | ETNYYRGGPAFDI | ETNYYRGGPAFDI |
| | (SEQ ID NO: 23) | (SEQ ID NO: 23) |
| VL CDR1 | RSSQSLLHSNGYNYLD | RSSQSLLHSNGYNYLD |
| | (SEQ ID NO: 24) | (SEQ ID NO: 24) |
| VL CDR2 | LGSNRAS | LGSNRAS |
| | (SEQ ID NO: 25) | (SEQ ID NO: 25) |
| VL CDR3 | MQVLGTPPWT | MQVLGTPPWT |
| | (SEQ ID NO: 26) | (SEQ ID NO: 26) |

VH
QVQLVQSGAEVKKPGASVKVSCKASGYTFTSYYMHWVRQAPGQGLEWMGI
INPSGGSTSYAQKFQGRVTMTRDTSTSTVYMELSSLRSEDTAVYYCARET
NYYRGGPAFDIWGQGTMVTVSS (SEQ ID NO: 27)
VL
DIVMTQSPLSLPVTPGEPASISCRSSQSLLHSNGYNYLDWYLQKPGQSPQ
LLIYLGSNRASGVPDRFSGSGSGTDFTLKISRVEAEDVGVYYCMQVLGTP
PWTFGGGTKVEIK (SEQ ID NO: 28)

In some instances, the anti-αvβ1 antibody comprises a VH comprising the three VH CDRs and a VL comprising the three VL CDRs of Exemplary Antibody 3. The six CDRs can be based on any definition known in the art such as, but not limited to, Kabat, Chothia, enhanced Chothia, contact, IMGT, or Honegger definitions. These CDRs can be determined, e.g., by using the AbYsis database.

In one instance, an anti-αvβ1 antibody of this disclosure comprises (i) a VH comprising a VHCDR1 comprising the amino acid sequence set forth in SEQ ID NO:4, a VHCDR2 comprising the amino acid sequence set forth in SEQ ID NO:6, and a VHCDR3 comprising the amino acid sequence set forth in SEQ ID NO: 23; and (ii) a VL comprising a VLCDR1 comprising the amino acid sequence set forth in SEQ ID NO:24, a VLCDR2 comprising the amino acid sequence set forth in SEQ ID NO:25, and a VLCDR3 comprising the amino acid sequence set forth in SEQ ID NO:26. In another instance, an anti-αvβ1 antibody of this disclosure comprises (i) a VH comprising a VHCDR1 comprising the amino acid sequence set forth in SEQ ID NO:3, a VHCDR2 comprising the amino acid sequence set forth in SEQ ID NO: 5, and a VHCDR3 comprising the amino acid sequence set forth in SEQ ID NO:23; and (ii) a VL comprising a VLCDR1 comprising the amino acid sequence set forth in SEQ ID NO:24, a VLCDR2 comprising the amino acid sequence set forth in SEQ ID NO:25, and a VLCDR3 comprising the amino acid sequence set forth in SEQ ID NO:26.

In some instances, the anti-αvβ1 antibody comprises a VH that is at least 75%, 80%, 85%, 90%, 91%, 92%, 93%, 94%, 95%, 96%, 97%, 98%, or 99% identical to the amino acid sequence set forth in SEQ ID NO:27. In some instances, the anti-αvβ1 antibody comprises a VL that is at least 75%, 80%, 85%, 90%, 91%, 92%, 93%, 94%, 95%, 96%, 97%, 98%, or 99% identical to the amino acid sequence set forth in SEQ ID NO:28. In one instance, the anti-αvβ1 antibody comprises a VH that is at least 85% identical to the amino acid sequence set forth in SEQ ID NO:27 and a VL that is at least 85% identical to the amino acid sequence set forth in SEQ ID NO:28. In another instance, the anti-αvβ1 antibody comprises a VH that is at least 90% identical to the amino acid sequence set forth in SEQ ID NO:27 and a VL that is at least 90% identical to the amino acid sequence set forth in SEQ ID NO:28. In yet another instance, the anti-αvβ1 antibody comprises a VH that is identical to the amino acid sequence set forth in SEQ ID NO:27 and a VL that is identical to the amino acid sequence set forth in SEQ ID NO:28.

In certain instances, an antibody of this disclosure that binds to αvβ1 is one that competes with or binds to the same epitope as a reference antibody with a VH having the amino acid sequence set forth in SEQ ID NO:27 and a VL having the amino acid sequence set forth in SEQ ID NO:28.

### Exemplary Antibody 4

Exemplary Antibody 4 specifically binds human αvβ1, and also bind to cynomolgus monkey, mouse, and rat αvβ1. Exemplary Antibody 4 is internalized. The amino acid sequences of the CDRs and the mature heavy chain variable region and light chain variable regions of Exemplary Antibody 4 are provided below.

| Domain | Kabat | Enhanced Chothia/AbM |
|---|---|---|
| VH CDR1 | SYYMH | FTFTSYYMH |
| | (SEQ ID NO: 3) | (SEQ ID NO: 29) |
| VH CDR2 | IINPSGGSTSYAQKFQG | IINPSGGSTS |
| | (SEQ ID NO: 5) | (SEQ ID NO: 6) |
| VH CDR3 | QQRHRRDYDYYYGMDV | QQRHRRDYDYYYGMDV |
| | (SEQ ID NO: 7) | (SEQ ID NO: 7) |
| VL CDR1 | RASQSVSSDYLA | RASQSVSSDYLA |
| | (SEQ ID NO: 8) | (SEQ ID NO: 8) |
| VL CDR2 | GASRRAT | GASRRAT |
| | (SEQ ID NO: 9) | (SEQ ID NO: 9) |
| VL CDR3 | QQAYSLPPT | QQAYSLPPT |
| | (SEQ ID NO: 10) | (SEQ ID NO: 10) |

VH
QVQLVESGGGLVQPGGSLRLSCAASGFTFTSYYMHWVRQAPGQGLEWMGI
INPSGGSTSYAQKFQGRVTMTRDTSTSTVYMELSSLRSEDTAVYYCARQQ
RHRRDYDYYYGMDVWGQGTTVTVSS (SEQ ID NO: 30)
VL
EIVLTQSPGTLSLSPGERATLSCRASQSVSSDYLAWYQQKPGQAPRLLIY
GASRRATGIPDRFSGSGSGTDFTLTISRLEPEDFAVYYCQQAYSLPPTFG
GGTKVEIK (SEQ ID NO: 12)

In some instances, the anti-αvβ1 antibody comprises a VH comprising the three VH CDRs and a VL comprising the three VL CDRs of Exemplary Antibody 4. The six CDRs can be based on any definition known in the art such as, but not limited to, Kabat, Chothia, enhanced Chothia, contact, IMGT, or Honegger definitions. These CDRs can be determined, e.g., by using the AbYsis database.

In one instance, an anti-αvβ1 antibody of this disclosure comprises (i) a VH comprising a VHCDR1 comprising the amino acid sequence set forth in SEQ ID NO:29, a VHCDR2 comprising the amino acid sequence set forth in SEQ ID NO: 6, and a VHCDR3 comprising the amino acid sequence set forth in SEQ ID NO: 7; and (ii) a VL comprising a VLCDR1 comprising the amino acid sequence set forth in SEQ ID NO:8, a VLCDR2 comprising the amino acid sequence set forth in SEQ ID NO: 9, and a VLCDR3 comprising the amino acid sequence set forth in SEQ ID NO: 10. In another instance, an anti-αvβ1 antibody of this disclosure comprises (i) a VH comprising a VHCDR1 comprising the amino acid sequence set forth in SEQ ID NO:3, a VHCDR2 comprising the amino acid sequence set forth in SEQ ID NO: 5, and a VHCDR3 comprising the amino acid sequence set forth in SEQ ID NO: 7; and (ii) a VL comprising a VLCDR1 comprising the amino acid sequence set forth in SEQ ID NO:8, a VLCDR2 comprising the amino acid sequence set forth in SEQ ID NO: 9, and a VLCDR3 comprising the amino acid sequence set forth in SEQ ID NO:10.

In some instances, the anti-αvβ1 antibody comprises a VH that is at least 75%, 80%, 85%, 90%, 91%, 92%, 93%, 94%, 95%, 96%, 97%, 98%, or 99% identical to the amino acid sequence set forth in SEQ ID NO:30. In some instances, the anti-αvβ1 antibody comprises a VL that is at least 75%, 80%, 85%, 90%, 91%, 92%, 93%, 94%, 95%, 96%, 97%, 98%, or 99% identical to the amino acid sequence set forth in SEQ ID NO:12. In one instance, the anti-αvβ1 antibody comprises a VH that is at least 85% identical to the amino acid sequence set forth in SEQ ID NO:30 and a VL that is at least 85% identical to the amino acid sequence set forth in SEQ ID NO:12. In another instance, the anti-αvβ1 antibody comprises a VH that is at least 90% identical to the amino acid sequence set forth in SEQ ID NO:30 and a VL that is at least 90% identical to the amino acid sequence set forth in SEQ ID NO: 12. In yet another instance, the anti-αvβ1 antibody comprises a VH that is identical to the amino acid sequence set forth in SEQ ID NO:30 and a VL that is identical to the amino acid sequence set forth in SEQ ID NO:12.

In certain instances, an antibody of this disclosure that binds to αvβ1 is one that competes with or binds to the same epitope as a reference antibody with a VH having the amino acid sequence set forth in SEQ ID NO:30 and a VL having the amino acid sequence set forth in SEQ ID NO: 12.

### Exemplary Antibody 5

Exemplary Antibody 5 specifically binds human αvβ1, and also bind to cynomolgus monkey, mouse, and rat αvβ1. Exemplary Antibody 5 is internalized. The amino acid sequences of the CDRs and the mature heavy chain variable region and light chain variable regions of Exemplary Antibody 5 are provided below.

| Domain | Kabat | Enhanced Chothia/AbM |
|---|---|---|
| VH CDR1 | YYGMH | FTFKYYGMH |
| | (SEQ ID NO: 31) | (SEQ ID NO: 32) |
| VH CDR2 | SIWYDGSNKKYADSVKG | SIWYDGSNKK |
| | (SEQ ID NO: 33) | (SEQ ID NO: 34) |
| VH CDR3 | GGPTRGDGTRVYYYGMDV | GGPTRGDGTRVYYYGMDV |
| | (SEQ ID NO: 17) | (SEQ ID NO: 17) |
| VL CDR1 | RASQSVSSNLA | RASQSVSSNLA |
| | (SEQ ID NO: 18) | (SEQ ID NO: 18) |
| VL CDR2 | SASTRAT | SASTRAT |
| | (SEQ ID NO: 19) | (SEQ ID NO: 19) |
| VL CDR3 | QQYYHHPFT | QQYYHHPFT |
| | (SEQ ID NO: 20) | (SEQ ID NO: 20) |

VH
EVQLVESGGGVVQPGRSLRLSCAASGFTFKYYGMHWVRQAPGKGLEWVAS
IWYDGSNKKYADSVKGRFTISRDNSKNTLYLQMNSLRAEDTAVYYCARGG
PTRGDGTRVYYYGMDVWGQGTTVTVSS (SEQ ID NO: 35)
VL
EIVMTQSPATLSVSPGERATLSCRASQSVSSNLAWYQQKPGQAPRLLIYS
ASTRATGIPARFSGSGSGTEFTLTISSLQSEDFAVYYCQQYYHHPFTFGG
GTKVEIK (SEQ ID NO: 22)

In some instances, the anti-αvβ1 antibody comprises a VH comprising the three VH CDRs and a VL comprising the three VL CDRs of Exemplary Antibody 5. The six CDRs can be based on any definition known in the art such as, but not limited to, Kabat, Chothia, enhanced Chothia, contact, IMGT, or Honegger definitions. These CDRs can be determined, e.g., by using the AbYsis database.

In one instance, an anti-αvβ1 antibody of this disclosure comprises (i) a VH comprising a VHCDR1 comprising the amino acid sequence set forth in SEQ ID NO:32, a VHCDR2 comprising the amino acid sequence set forth in SEQ ID NO: 34, and a VHCDR3 comprising the amino acid sequence set forth in SEQ ID NO: 17; and (ii) a VL comprising a VLCDR1 comprising the amino acid sequence set forth in SEQ ID NO:18, a VLCDR2 comprising the amino acid sequence set forth in SEQ ID NO: 19, and a VLCDR3 comprising the amino acid sequence set forth in SEQ ID NO:20. In another instance, an anti-αvβ1 antibody of this disclosure comprises (i) a VH comprising a VHCDR1 comprising the amino acid sequence set forth in SEQ ID NO:31, a VHCDR2 comprising the amino acid sequence set forth in SEQ ID NO:33, and a VHCDR3 comprising the amino acid sequence set forth in SEQ ID NO: 17; and (ii) a VL comprising a VLCDR1 comprising the amino acid sequence set forth in SEQ ID NO:18, a VLCDR2 comprising the amino acid sequence set forth in SEQ ID NO: 19, and a VLCDR3 comprising the amino acid sequence set forth in SEQ ID NO:20.

In some instances, the anti-αvβ1 antibody comprises a VH that is at least 75%, 80%, 85%, 90%, 91%, 92%, 93%, 94%, 95%, 96%, 97%, 98%, or 99% identical to the amino acid sequence set forth in SEQ ID NO:35. In some instances, the anti-αvβ1 antibody comprises a VL that is at least 75%, 80%, 85%, 90%, 91%, 92%, 93%, 94%, 95%, 96%, 97%, 98%, or 99% identical to the amino acid sequence set forth in SEQ ID NO:22. In one instance, the anti-αvβ1 antibody comprises a VH that is at least 85% identical to the amino acid sequence set forth in SEQ ID NO:35 and a VL that is at least 85% identical to the amino acid sequence set forth in SEQ ID NO:22. In another instance, the anti-αvβ1 antibody comprises a VH that is at least 90% identical to the amino acid sequence set forth in SEQ ID NO:35 and a VL that is at least 90% identical to the amino acid sequence set forth in SEQ ID NO:22. In yet another instance, the anti-αvβ1 antibody comprises a VH that is identical to the amino acid sequence set forth in SEQ ID NO:35 and a VL that is identical to the amino acid sequence set forth in SEQ ID NO:22.

In certain instances, an antibody of this disclosure that binds to αvβ1 is one that competes with or binds to the same epitope as a reference antibody with a VH having the amino acid sequence set forth in SEQ ID NO:35 and a VL having the amino acid sequence set forth in SEQ ID NO:22.

### Exemplary Antibody 6

Exemplary Antibody 6 specifically binds human αvβ1. The amino acid sequences of the CDRs and the mature heavy chain variable region and light chain variable regions of Exemplary Antibody 6 are provided below.

| Domain | Kabat | Enhanced Chothia/AbM |
|---|---|---|
| VH CDR1 | DYYMS | FTFSDYYMS |
| | (SEQ ID NO: 36) | (SEQ ID NO: 37) |
| VH CDR2 | YISSSGSTIYYADSVKG | YISSSGSTIY |
| | (SEQ ID NO: 38) | (SEQ ID NO: 39) |
| VH CDR3 | GGRNRGDSSLSGIDV | GGRNRGDSSLSGIDV |
| | (SEQ ID NO: 40) | (SEQ ID NO: 40) |
| VL CDR1 | RASQSINSYLN | RASQSINSYLN |
| | (SEQ ID NO: 41) | (SEQ ID NO: 41) |
| VL CDR2 | AASSLQS | AASSLQS |
| | (SEQ ID NO: 42) | (SEQ ID NO: 42) |
| VL CDR3 | QQQYSDIT | QQQYSDIT |
| | (SEQ ID NO: 43) | (SEQ ID NO: 43) |

VH
QVQLVESGGGLVKPGGSLRLSCAASGFTFSDYYMSWIRQAPGKGLEWVSY
ISSSGSTIYYADSVKGRFTISRDNAKNSLYLQMNSLRAEDTAVYYCARGG
RNRGDSSLSGIDVWGQGTTVTVSS (SEQ ID NO: 44)
VL
DIQMTQSPSSLSASVGDRVTITCRASQSINSYLNWYQQKPGKAPKLLIYA
ASSLQSGVPSRFSGSGSGTDFTLTISSLQPEDFATYYCQQQYSDITFGGG
TKVEIK (SEQ ID NO: 45)

In some instances, the anti-αvβ1 antibody comprises a VH comprising the three VH CDRs and a VL comprising the three VL CDRs of Exemplary Antibody 6. The six CDRs can be based on any definition known in the art such as, but not limited to, Kabat, Chothia, enhanced Chothia, contact, IMGT, or Honegger definitions. These CDRs can be determined, e.g., by using the AbYsis database.

In one instance, an anti-αvβ1 antibody of this disclosure comprises (i) a VH comprising a VHCDR1 comprising the amino acid sequence set forth in SEQ ID NO:37, a VHCDR2 comprising the amino acid sequence set forth in SEQ ID NO:39, and a VHCDR3 comprising the amino acid sequence set forth in SEQ ID NO:40; and (ii) a VL comprising a VLCDR1 comprising the amino acid sequence set forth in SEQ ID NO:41, a VLCDR2 comprising the amino acid sequence set forth in SEQ ID NO:42, and a VLCDR3 comprising the amino acid sequence set forth in SEQ ID NO:43. In another instance, an anti-αvβ1 antibody of this disclosure comprises (i) a VH comprising a VHCDR1 comprising the amino acid sequence set forth in SEQ ID NO:36, a VHCDR2 comprising the amino acid sequence set forth in SEQ ID NO:38, and a VHCDR3 comprising the amino acid sequence set forth in SEQ ID NO:40; and (ii) a VL comprising a VLCDR1 comprising the amino acid sequence set forth in SEQ ID NO:41, a VLCDR2 comprising the amino acid sequence set forth in SEQ ID NO:42, and a VLCDR3 comprising the amino acid sequence set forth in SEQ ID NO:43.

In some instances, the anti-αvβ1 antibody comprises a VH that is at least 75%, 80%, 85%, 90%, 91%, 92%, 93%, 94%, 95%, 96%, 97%, 98%, or 99% identical to the amino acid sequence set forth in SEQ ID NO:44. In some instances, the anti-αvβ1 antibody comprises a VL that is at least 75%, 80%, 85%, 90%, 91%, 92%, 93%, 94%, 95%, 96%, 97%, 98%, or 99% identical to the amino acid sequence set forth in SEQ ID NO:45. In one instance, the anti-αvβ1 antibody comprises a VH that is at least 85% identical to the amino acid sequence set forth in SEQ ID NO:44 and a VL that is at least 85% identical to the amino acid sequence set forth in SEQ ID NO:45. In another instance, the anti-αvβ1 antibody comprises a VH that is at least 90% identical to the amino acid sequence set forth in SEQ ID NO:44 and a VL that is at least 90% identical to the amino acid sequence set forth in SEQ ID NO:45. In yet another instance, the anti-αvβ1 antibody comprises a VH that is identical to the amino acid sequence set forth in SEQ ID NO:44 and a VL that is identical to the amino acid sequence set forth in SEQ ID NO:45.

In certain instances, an antibody of this disclosure that binds to αvβ1 is one that competes with or binds to the same epitope as a reference antibody with a VH having the amino acid sequence set forth in SEQ ID NO:44 and a VL having the amino acid sequence set forth in SEQ ID NO:45.

### Exemplary Antibody 7

Exemplary Antibody 7 specifically binds human αvβ1. The amino acid sequences of the CDRs and the mature heavy chain variable region and light chain variable regions of Exemplary Antibody 7 are provided below.

| Domain | Kabat | Enhanced Chothia/AbM |
|---|---|---|
| VH CDR1 | DYYMS | FTFSDYYMS |
| | (SEQ ID NO: 36) | (SEQ ID NO: 37) |
| VH CDR2 | YISSSGSTIYYADSVKG | YISSSGSTIY |
| | (SEQ ID NO: 38) | (SEQ ID NO: 39) |
| VH CDR3 | GGPSRGDALAEYFQH | GGPSRGDALAEYFQH |
| | (SEQ ID NO: 46) | (SEQ ID NO: 46) |
| VL CDR1 | RASQSVSSNLA | RASQSVSSNLA |
| | (SEQ ID NO: 18) | (SEQ ID NO: 18) |
| VL CDR2 | GASTRAT | GASTRAT |
| | (SEQ ID NO: 47) | (SEQ ID NO: 47) |
| VL CDR3 | QQLVNYPPIT | QQLVNYPPIT |
| | (SEQ ID NO: 48) | (SEQ ID NO: 48) |

VH
QVQLVESGGGLVKPGGSLRLSCAASGFTFSDYYMSWIRQAPGKGLEWVSY
ISSSGSTIYYADSVKGRFTISRDNAKNSLYLQMNSLRAEDTAVYYCARGG
PSRGDALAEYFQHWGQGTTVTVSS (SEQ ID NO: 49)
VL
EIVMTQSPATLSVSPGERATLSCRASQSVSSNLAWYQQKPGQAPRLLIYG
ASTRATGIPARFSGSGSGTEFTLTISSLQSEDFAVYYCQQLVNYPPITFG
GGTKVEIK (SEQ ID NO: 50)

In some instances, the anti-αvβ1 antibody comprises a VH comprising the three VH CDRs and a VL comprising the three VL CDRs of Exemplary Antibody 7. The six CDRs can be based on any definition known in the art such as, but not limited to, Kabat, Chothia, enhanced Chothia, contact, IMGT, or Honegger definitions. These CDRs can be determined, e.g., by using the AbYsis database.

In one instance, an anti-αvβ1 antibody of this disclosure comprises (i) a VH comprising a VHCDR1 comprising the amino acid sequence set forth in SEQ ID NO:37, a VHCDR2 comprising the amino acid sequence set forth in SEQ ID NO:39, and a VHCDR3 comprising the amino acid sequence set forth in SEQ ID NO:46; and (ii) a VL comprising a VLCDR1 comprising the amino acid sequence set forth in SEQ ID NO:18, a VLCDR2 comprising the amino acid sequence set forth in SEQ ID NO:47, and a VLCDR3 comprising the amino acid sequence set forth in SEQ ID NO:48. In another instance, an anti-αvβ1 antibody of this disclosure comprises (i) a VH comprising a VHCDR1 comprising the amino acid sequence set forth in SEQ ID NO:36, a VHCDR2 comprising the amino acid sequence set forth in SEQ ID NO:38, and a VHCDR3 comprising the amino acid sequence set forth in SEQ ID NO:46; and (ii) a VL comprising a VLCDR1 comprising the amino acid sequence set forth in SEQ ID NO:18, a VLCDR2 comprising the amino acid sequence set forth in SEQ ID NO:47, and a VLCDR3 comprising the amino acid sequence set forth in SEQ ID NO:48.

In some instances, the anti-αvβ1 antibody comprises a VH that is at least 75%, 80%, 85%, 90%, 91%, 92%, 93%, 94%, 95%, 96%, 97%, 98%, or 99% identical to the amino acid sequence set forth in SEQ ID NO:49. In some instances, the anti-αvβ1 antibody comprises a VL that is at least 75%, 80%, 85%, 90%, 91%, 92%, 93%, 94%, 95%, 96%, 97%, 98%, or 99% identical to the amino acid sequence set forth in SEQ ID NO:50. In one instance, the anti-αvβ1 antibody comprises a VH that is at least 85% identical to the amino acid sequence set forth in SEQ ID NO:49 and a VL that is at least 85% identical to the amino acid sequence set forth in SEQ ID NO:50. In another instance, the anti-αvβ1 antibody comprises a VH that is at least 90% identical to the amino acid sequence set forth in SEQ ID NO:49 and a VL that is at least 90% identical to the amino acid sequence set forth in SEQ ID NO:50. In yet another instance, the anti-αvβ1 antibody comprises a VH that is identical to the amino acid sequence set forth in SEQ ID NO:49 and a VL that is identical to the amino acid sequence set forth in SEQ ID NO:50.

In certain instances, an antibody of this disclosure that binds to αvβ1 is one that competes with or binds to the same epitope as a reference antibody with a VH having the amino acid sequence set forth in SEQ ID NO:49 and a VL having the amino acid sequence set forth in SEQ ID NO:50.

### Exemplary Antibody 8

Exemplary Antibody 8 specifically binds human αvβ1. The amino acid sequences of the CDRs and the mature heavy chain variable region and light chain variable regions of Exemplary Antibody 8 are provided below.

| Domain | Kabat | Enhanced Chothia/AbM |
|---|---|---|
| VH CDR1 | DYSMN | FTFYDYSMN |
| | (SEQ ID NO: 51) | (SEQ ID NO: 52) |
| VH CDR2 | YISSSSSTIYYADSVKG | YISSSSSTIY |
| | (SEQ ID NO: 53) | (SEQ ID NO: 54) |
| VH CDR3 | GLWSTEVRYYYMDV | GLWSTEVRYYYMDV |
| | (SEQ ID NO: 55) | (SEQ ID NO: 55) |
| VL CDR1 | RASQSVSSNLA | RASQSVSSNLA |
| | (SEQ ID NO: 18) | (SEQ ID NO: 18) |
| VL CDR2 | SASTRAT | SASTRAT |
| | (SEQ ID NO: 19) | (SEQ ID NO: 19) |
| VL CDR3 | QQSNAWPFT | QQSNAWPFT |
| | (SEQ ID NO: 56) | (SEQ ID NO: 56) |

VH
EVQLVESGGGLVQPGGSLRLSCAASGFTFYDYSMNWVRQAPGKGLEWVSY
ISSSSSTIYYADSVKGRFTISRDNAKNSLYLQMNSLRAEDTAVYYCARGL
WSTEVRYYYMDVWGKGTTVTVSS (SEQ ID NO: 57)
VL
EIVMTQSPATLSVSPGERATLSCRASQSVSSNLAWYQQKPGQAPRLLIYS
ASTRATGIPARFSGSGSGTEFTLTISSLQSEDFAVYYCQQSNAWPFTFGG
GTKVEIK (SEQ ID NO: 58)

In some instances, the anti-αvβ1 antibody comprises a VH comprising the three VH CDRs and a VL comprising the three VL CDRs of Exemplary Antibody 8. The six CDRs can be based on any definition known in the art such as, but not limited to, Kabat, Chothia, enhanced Chothia, contact, IMGT, or Honegger definitions. These CDRs can be determined, e.g., by using the AbYsis database.

In one instance, an anti-αvβ1 antibody of this disclosure comprises (i) a VH comprising a VHCDR1 comprising the amino acid sequence set forth in SEQ ID NO:52, a VHCDR2 comprising the amino acid sequence set forth in SEQ ID NO:54, and a VHCDR3 comprising the amino acid sequence set forth in SEQ ID NO:55; and (ii) a VL comprising a VLCDR1 comprising the amino acid sequence set forth in SEQ ID NO:18, a VLCDR2 comprising the amino acid sequence set forth in SEQ ID NO: 19, and a VLCDR3 comprising the amino acid sequence set forth in SEQ ID NO: 56. In another instance, an anti-αvβ1 antibody of this disclosure comprises (i) a VH comprising a VHCDR1 comprising the amino acid sequence set forth in SEQ ID NO:51, a VHCDR2 comprising the amino acid sequence set forth in SEQ ID NO:53, and a VHCDR3 comprising the amino acid sequence set forth in SEQ ID NO:55; and (ii) a VL comprising a VLCDR1 comprising the amino acid sequence set forth in SEQ ID NO:18, a VLCDR2 comprising the amino acid sequence set forth in SEQ ID NO:19, and a VLCDR3 comprising the amino acid sequence set forth in SEQ ID NO:56.

In some instances, the anti-αvβ1 antibody comprises a VH that is at least 75%, 80%, 85%, 90%, 91%, 92%, 93%, 94%, 95%, 96%, 97%, 98%, or 99% identical to the amino acid sequence set forth in SEQ ID NO:57. In some instances, the anti-αvβ1 antibody comprises a VL that is at least 75%, 80%, 85%, 90%, 91%, 92%, 93%, 94%, 95%, 96%, 97%, 98%, or 99% identical to the amino acid sequence set forth in SEQ ID NO:58. In one instance, the anti-αvβ1 antibody comprises a VH that is at least 85% identical to the amino acid sequence set forth in SEQ ID NO:57 and a VL that is at least 85% identical to the amino acid sequence set forth in SEQ ID NO:58. In another instance, the anti-αvβ1 antibody comprises a VH that is at least 90% identical to the amino acid sequence set forth in SEQ ID NO:57 and a VL that is at least 90% identical to the amino acid sequence set forth in SEQ ID NO:58. In yet another instance, the anti-αvβ1 antibody comprises a VH that is identical to the amino acid sequence set forth in SEQ ID NO:57 and a VL that is identical to the amino acid sequence set forth in SEQ ID NO:58.

In certain instances, an antibody of this disclosure that binds to αvβ1 is one that competes with or binds to the same epitope as a reference antibody with a VH having the amino acid sequence set forth in SEQ ID NO:57 and a VL having the amino acid sequence set forth in SEQ ID NO:58.

### Exemplary Antibody 9

Exemplary Antibody 9 specifically binds human αvβ1. The amino acid sequences of the CDRs and the mature heavy chain variable region and light chain variable regions of Exemplary Antibody 9 are provided below.

| Domain | Kabat | Enhanced Chothia/AbM |
|---|---|---|
| VH CDR1 | DYSMH | FTFDDYSMH |
| | (SEQ ID NO: 59) | (SEQ ID NO: 60) |
| VH CDR2 | YISSSGSTIYYADSVKG | YISSSGSTIY |
| | (SEQ ID NO: 38) | (SEQ ID NO: 39) |
| VH CDR3 | GLWSTEVRYYYMDV | GLWSTEVRYYYMDV |
| | (SEQ ID NO: 55) | (SEQ ID NO: 55) |
| VL CDR1 | RASQSVSSNLA | RASQSVSSNLA |
| | (SEQ ID NO: 18) | (SEQ ID NO: 18) |
| VL CDR2 | SASTRAT | SASTRAT |
| | (SEQ ID NO: 19) | (SEQ ID NO: 19) |
| VL CDR3 | QQSNAWPFT | QQSNAWPFT |
| | (SEQ ID NO: 56) | (SEQ ID NO: 56) |

VH
EVQLVESGGGLVQPGGSLRLSCAASGFTFDDYSMHWVRQAPGKGLEWVSY
ISSSGSTIYYADSVKGRFTISRDNAKNSLYLQMNSLRAEDTAVYYCARGL
WSTEVRYYYMDVWGKGTTVTVSS (SEQ ID NO: 61)
VL
EIVMTQSPATLSVSPGERATLSCRASQSVSSNLAWYQQKPGQAPRLLIYS
ASTRATGIPARFSGSGSGTEFTLTISSLQSEDFAVYYCQQSNAWPFTFGG
GTKVEIK (SEQ ID NO: 58)

In some instances, the anti-αvβ1 antibody comprises a VH comprising the three VH CDRs and a VL comprising the three VL CDRs of Exemplary Antibody 9. The six CDRs can be based on any definition known in the art such as, but not limited to, Kabat, Chothia, enhanced Chothia, contact, IMGT, or Honegger definitions. These CDRs can be determined, e.g., by using the AbYsis database.

In one instance, an anti-αvβ1 antibody of this disclosure comprises (i) a VH comprising a VHCDR1 comprising the amino acid sequence set forth in SEQ ID NO:60, a VHCDR2 comprising the amino acid sequence set forth in SEQ ID NO:39, and a VHCDR3 comprising the amino acid sequence set forth in SEQ ID NO:55; and (ii) a VL comprising a VLCDR1 comprising the amino acid sequence set forth in SEQ ID NO:18, a VLCDR2 comprising the amino acid sequence set forth in SEQ ID NO: 19, and a VLCDR3 comprising the amino acid sequence set forth in SEQ ID NO: 56. In another instance, an anti-αvβ1 antibody of this disclosure comprises (i) a VH comprising a VHCDR1 comprising the amino acid sequence set forth in SEQ ID NO:59, a VHCDR2 comprising the amino acid sequence set forth in SEQ ID NO:38, and a VHCDR3 comprising the amino acid sequence set forth in SEQ ID NO:55; and (ii) a VL comprising a VLCDR1 comprising the amino acid sequence set forth in SEQ ID NO:18, a VLCDR2 comprising the amino acid sequence set forth in SEQ ID NO:19, and a VLCDR3 comprising the amino acid sequence set forth in SEQ ID NO:56.

In some instances, the anti-αvβ1 antibody comprises a VH that is at least 75%, 80%, 85%, 90%, 91%, 92%, 93%, 94%, 95%, 96%, 97%, 98%, or 99% identical to the amino acid sequence set forth in SEQ ID NO:61. In some instances, the anti-αvβ1 antibody comprises a VL that is at least 75%, 80%, 85%, 90%, 91%, 92%, 93%, 94%, 95%, 96%, 97%, 98%, or 99% identical to the amino acid sequence set forth in SEQ ID NO:58. In one instance, the anti-αvβ1 antibody comprises a VH that is at least 85% identical to the amino acid sequence set forth in SEQ ID NO:61 and a VL that is at least 85% identical to the amino acid sequence set forth in SEQ ID NO:58. In another instance, the anti-αvβ1 antibody comprises a VH that is at least 90% identical to the amino acid sequence set forth in SEQ ID NO:61 and a VL that is at least 90% identical to the amino acid sequence set forth in SEQ ID NO:58. In yet another instance, the anti-αvβ1 antibody comprises a VH that is identical to the amino acid sequence set forth in SEQ ID NO:61 and a VL that is identical to the amino acid sequence set forth in SEQ ID NO:58.

In certain instances, an antibody of this disclosure that binds to αvβ1 is one that competes with or binds to the same epitope as a reference antibody with a VH having the amino acid sequence set forth in SEQ ID NO:61 and a VL having the amino acid sequence set forth in SEQ ID NO:58.

### Exemplary Antibody 10

Exemplary Antibody 10 specifically binds human αvβ1. The amino acid sequences of the CDRs and the mature heavy chain variable region and light chain variable regions of Exemplary Antibody 10 are provided below.

| Domain | Kabat | Enhanced Chothia/AbM |
|---|---|---|
| VH CDR1 | EYSMF | FTFGEYSMF |
| | (SEQ ID NO: 62) | (SEQ ID NO: 63) |
| VH CDR2 | YISSSSSTIYYADSVKG | YISSSSSTIY |
| | (SEQ ID NO: 53) | (SEQ ID NO: 54) |
| VH CDR3 | GLWSTEVRYYYMDV | GLWSTEVRYYYMDV |
| | (SEQ ID NO: 55) | (SEQ ID NO: 55) |
| VL CDR1 | RASQSVSSNLA | RASQSVSSNLA |
| | (SEQ ID NO: 18) | (SEQ ID NO: 18) |
| VL CDR2 | SASTRAT | SASTRAT |
| | (SEQ ID NO: 19) | (SEQ ID NO: 19) |
| VL CDR3 | QQSNAWPFT | QQSNAWPFT |
| | (SEQ ID NO: 56) | (SEQ ID NO: 56) |

VH
EVQLVESGGGLVQPGGSLRLSCAASGFTFGEYSMFWVRQAPGKGLEWVSY
ISSSSSTIYYADSVKGRFTISRDNAKNSLYLQMNSLRAEDTAVYYCARGL
WSTEVRYYYMDVWGKGTTVTVSS (SEQ ID NO: 64)
VL
EIVMTQSPATLSVSPGERATLSCRASQSVSSNLAWYQQKPGQAPRLLIYS
ASTRATGIPARFSGSGSGTEFTLTISSLQSEDFAVYYCQQSNAWPFTFGG
GTKVEIK (SEQ ID NO: 58)

In some instances, the anti-αvβ1 antibody comprises a VH comprising the three VH CDRs and a VL comprising the three VL CDRs of Exemplary Antibody 10. The six CDRs can be based on any definition known in the art such as, but not limited to, Kabat, Chothia, enhanced Chothia, contact, IMGT, or Honegger definitions. These CDRs can be determined, e.g., by using the AbYsis database.

In one instance, an anti-αvβ1 antibody of this disclosure comprises (i) a VH comprising a VHCDR1 comprising the amino acid sequence set forth in SEQ ID NO:63, a VHCDR2 comprising the amino acid sequence set forth in SEQ ID NO:54, and a VHCDR3 comprising the amino acid sequence set forth in SEQ ID NO:55; and (ii) a VL comprising a VLCDR1 comprising the amino acid sequence set forth in SEQ ID NO:18, a VLCDR2 comprising the amino acid sequence set forth in SEQ ID NO:19, and a VLCDR3 comprising the amino acid sequence set forth in SEQ ID NO:56. In another instance, an anti-αvβ1 antibody of this disclosure comprises (i) a VH comprising a VHCDR1 comprising the amino acid sequence set forth in SEQ ID NO:62, a VHCDR2 comprising the amino acid sequence set forth in SEQ ID NO:53, and a VHCDR3 comprising the amino acid sequence set forth in SEQ ID NO:55; and (ii) a VL comprising a VLCDR1 comprising the amino acid sequence set forth in SEQ ID NO:18, a VLCDR2 comprising the amino acid sequence set forth in SEQ ID NO:19, and a VLCDR3 comprising the amino acid sequence set forth in SEQ ID NO:56.

In some instances, the anti-αvβ1 antibody comprises a VH that is at least 75%, 80%, 85%, 90%, 91%, 92%, 93%, 94%, 95%, 96%, 97%, 98%, or 99% identical to the amino acid sequence set forth in SEQ ID NO:64. In some instances, the anti-αvβ1 antibody comprises a VL that is at least 75%, 80%, 85%, 90%, 91%, 92%, 93%, 94%, 95%, 96%, 97%, 98%, or 99% identical to the amino acid sequence set forth in SEQ ID NO:58. In one instance, the anti-αvβ1 antibody comprises a VH that is at least 85% identical to the amino acid sequence set forth in SEQ ID NO:64 and a VL that is at least 85% identical to the amino acid sequence set forth in SEQ ID NO:58. In another instance, the anti-αvβ1 antibody comprises a VH that is at least 90% identical to the amino acid sequence set forth in SEQ ID NO:64 and a VL that is at least 90% identical to the amino acid sequence set forth in SEQ ID NO:58. In yet another instance, the anti-αvβ1 antibody comprises a VH that is identical to the amino acid sequence set forth in SEQ ID NO:64 and a VL that is identical to the amino acid sequence set forth in SEQ ID NO:58.

In certain instances, an antibody of this disclosure that binds to αvβ1 is one that competes with or binds to the same epitope as a reference antibody with a VH having the amino acid sequence set forth in SEQ ID NO:64 and a VL having the amino acid sequence set forth in SEQ ID NO:58.

B. Group II (Antibodies that Bind to Both αvβ1 and αvβ6 Integrin but not to Other Integrins)

This disclosure further provides antibodies that bind to both αvβ1 and αvβ6 integrins. In some instances, the antibodies do not bind to other integrins. In some instances, the antibodies do not bind to RGD-binding integrins (e.g. αvβ3, αvβ5, αvβ8, α5β1, α8β1, and αIIBβ3) other than αvβ1 and αvβ6 integrins. The antibodies of Group II all bind to human αvβ1 and human αvβ6 integrins. Such antibodies include the sequences of Exemplary Antibodies 11-14, which bind with high affinity of KD≤20 nM (bivalent affinity) to human αvβ1, and with affinity of 100 nM (bivalent affinity) to human αvβ6.

### Exemplary Antibody 11

Exemplary Antibody 11 specifically binds to human αvβ1 and αvβ6 but not other integrins (e.g., other RGD family of integrins). The amino acid sequences of the CDRs and the mature heavy chain variable region and light chain variable regions of Exemplary Antibody 11 are provided below.

| Domain | Kabat | Enhanced Chothia/AbN |
|---|---|---|
| VH CDR1 | DYYMS | FTFSDYYMS |
| | (SEQ ID NO: 36) | (SEQ ID NO: 37) |
| VH CDR2 | YISSSGSTIYYADSVKG | YISSSGSTIY |
| | (SEQ ID NO: 38) | (SEQ ID NO: 39) |
| VH CDR3 | GGRNRGDSSLSGIDV | GGRNRGDSSLSGIDV |
| | (SEQ ID NO: 40) | (SEQ ID NO: 40) |
| VL CDR1 | RASQSISSYLN | RASQSISSYLN |
| | (SEQ ID NO: 65) | (SEQ ID NO: 65) |
| VL CDR2 | GASSLQS | GASSLQS |
| | (SEQ ID NO: 66) | (SEQ ID NO: 66) |
| VL CDR3 | QQQYDDIT | QQQYDDIT |
| | (SEQ ID NO: 67) | (SEQ ID NO: 67) |

VH
QVQLVESGGGLVKPGGSLRLSCAASGFTFSDYYMSWIRQAPGKGLEWVSY
ISSSGSTIYYADSVKGRFTISRDNAKNSLYLQMNSLRAEDTAVYYCARGG
RNRGDSSLSGIDVWGQGTTVTVSS (SEQ ID NO: 44)
VL
DIQMTQSPSSLSASVGDRVTITCRASQSISSYLNWYQQKPGKAPKLLIYG
ASSLQSGVPSRFSGSGSGTDFTLTISSLQPEDFATYYCQQQYDDITFGGG
TKVEIK (SEQ ID NO: 68)

In some instances, the antibody that binds to both human αvβ1 and αvβ6 comprises a VH comprising the three VH CDRs and a VL comprising the three VL CDRs of Exemplary Antibody 11. The six CDRs can be based on any definition known in the art such as, but not limited to, Kabat, Chothia, enhanced Chothia, contact, IMGT, or Honegger definitions. These CDRs can be determined, e.g., by using the AbYsis database.

In one instance, an antibody that binds to both human αvβ1 and αvβ6 comprises (i) a VH comprising a VHCDR1 comprising the amino acid sequence set forth in SEQ ID NO:37, a VHCDR2 comprising the amino acid sequence set forth in SEQ ID NO:39, and a VHCDR3 comprising the amino acid sequence set forth in SEQ ID NO:40; and (ii) a VL comprising a VLCDR1 comprising the amino acid sequence set forth in SEQ ID NO: 65, a VLCDR2 comprising the amino acid sequence set forth in SEQ ID NO: 66, and a VLCDR3 comprising the amino acid sequence set forth in SEQ ID NO: 67. In another instance, an antibody that binds to both human αvβ1 and αvβ6 comprises (i) a VH comprising a VHCDR1 comprising the amino acid sequence set forth in SEQ ID NO:36, a VHCDR2 comprising the amino acid sequence set forth in SEQ ID NO: 38, and a VHCDR3 comprising the amino acid sequence set forth in SEQ ID NO: 40; and (ii) a VL comprising a VLCDR1 comprising the amino acid sequence set forth in SEQ ID NO:65, a VLCDR2 comprising the amino acid sequence set forth in SEQ ID NO:66, and a VLCDR3 comprising the amino acid sequence set forth in SEQ ID NO:67.

In some instances, an antibody that binds to both human αvβ1 and αvβ6 comprises a VH that is at least 75%, 80%, 85%, 90%, 91%, 92%, 93%, 94%, 95%, 96%, 97%, 98%, or 99% identical to the amino acid sequence set forth in SEQ ID NO:44. In some instances, the antibody that binds to both human αvβ1 and αvβ6 comprises a VL that is at least 75%, 80%, 85%, 90%, 91%, 92%, 93%, 94%, 95%, 96%, 97%, 98%, or 99% identical to the amino acid sequence set forth in SEQ ID NO:68. In one instance, the antibody that binds to both human αvβ1 and αvβ6 comprises a VH that is at least 85% identical to the amino acid sequence set forth in SEQ ID NO:44 and a VL that is at least 85% identical to the amino acid sequence set forth in SEQ ID NO:68. In another instance, the a antibody that binds to both human αvβ1 and αvβ6 comprises a VH that is at least 90% identical to the amino acid sequence set forth in SEQ ID NO:44 and a VL that is at least 90% identical to the amino acid sequence set forth in SEQ ID NO:68. In yet another instance, the antibody that binds to both human αvβ1 and αvβ6 comprises a VH that is identical to the amino acid sequence set forth in SEQ ID NO:44 and a VL that is identical to the amino acid sequence set forth in SEQ ID NO:68.

In certain instances, an antibody that binds to both human αvβ1 and αvβ6 is one that competes with or binds to the same epitope as a reference antibody with a VH having the amino acid sequence set forth in SEQ ID NO:44 and a VL having the amino acid sequence set forth in SEQ ID NO:68.

### Exemplary Antibody 12

Exemplary Antibody 12 specifically binds to human αvβ1 and αvβ6 but not other integrins (e.g., other RGD family of integrins). The amino acid sequences of the CDRs and the mature heavy chain variable region and light chain variable regions of Exemplary Antibody 12 are provided below.

| | | Enhanced |
|---|---|---|
| Domain | Kabat | Chothia/AbM |
| VH CDR1 | DYYMS | FTFSDYYMS |
| | (SEQ ID NO: 36) | (SEQ ID NO: 37) |
| VH CDR2 | YISSSGSTIYYADSVKG | YISSSGSTIY |
| | (SEQ ID NO: 38) | (SEQ ID NO: 39) |
| VH CDR3 | GGRNRGDSSLSGIDV | GGRNRGDSSLSGIDV |
| | (SEQ ID NO: 40) | (SEQ ID NO: 40) |
| VL CDR1 | RASQSISSYLN | RASQSISSYLN |
| | (SEQ ID NO: 65) | (SEQ ID NO: 65) |
| VL CDR2 | GASSLQS | GASSLQS |
| | (SEQ ID NO: 66) | (SEQ ID NO: 66) |
| VL CDR3 | QQQYIDIT | QQQYIDIT |
| | (SEQ ID NO: 69) | (SEQ ID NO: 69) |

VH
(SEQ ID NO: 44)
QVQLVESGGGLVKPGGSLRLSCAASGFTFSDYYMSWIRQAPGK
GLEWVSYISSSGSTIYYADSVKGRFTISRDNAKNSLYLQMNSL
RAEDTAVYYCARGGRNRGDSSLSGIDVWGQGTTVTVSS
VL
(SEQ ID NO: 70)
DIQMTQSPSSLSASVGDRVTITCRASQSISSYLNWYQQKPGKA
PKLLIYGASSLQSGVPSRFSGSGSGTDFTLTISSLQPEDFATY
YCQQQYIDITFGGGTKVEIK

In some instances, the antibody that binds to both human αvβ1 and αvβ6 comprises a VH comprising the three VH CDRs and a VL comprising the three VL CDRs of Exemplary Antibody 12. The six CDRs can be based on any definition known in the art such as, but not limited to, Kabat, Chothia, enhanced Chothia, contact, IMGT, or Honegger definitions.

In one instance, an antibody that binds to both human αvβ1 and αvβ6 comprises (i) a VH comprising a VHCDR1 comprising the amino acid sequence set forth in SEQ ID NO:37, a VHCDR2 comprising the amino acid sequence set forth in SEQ ID NO:39, and a VHCDR3 comprising the amino acid sequence set forth in SEQ ID NO:40; and (ii) a VL comprising a VLCDR1 comprising the amino acid sequence set forth in SEQ ID NO: 65, a VLCDR2 comprising the amino acid sequence set forth in SEQ ID NO: 66, and a VLCDR3 comprising the amino acid sequence set forth in SEQ ID NO: 69. In another instance, an antibody that binds to both human αvβ1 and αvβ6 comprises (i) a VH comprising a VHCDR1 comprising the amino acid sequence set forth in SEQ ID NO:36, a VHCDR2 comprising the amino acid sequence set forth in SEQ ID NO:38, and a VHCDR3 comprising the amino acid sequence set forth in SEQ ID NO: 40; and (ii) a VL comprising a VLCDR1 comprising the amino acid sequence set forth in SEQ ID NO:65, a VLCDR2 comprising the amino acid sequence set forth in SEQ ID NO: 66, and a VLCDR3 comprising the amino acid sequence set forth in SEQ ID NO: 69.

In some instances, an antibody that binds to both human αvβ1 and αvβ6 comprises a VH that is at least 75%, 80%, 85%, 90%, 91%, 92%, 93%, 94%, 95%, 96%, 97%, 98%, or 99% identical to the amino acid sequence set forth in SEQ ID NO:44. In some instances, the antibody that binds to both human αvβ1 and αvβ6 comprises a VL that is at least 75%, 80%, 85%, 90%, 91%, 92%, 93%, 94%, 95%, 96%, 97%, 98%, or 99% identical to the amino acid sequence set forth in SEQ ID NO:70. In one instance, the antibody that binds to both human αvβ1 and αvβ6 comprises a VH that is at least 85% identical to the amino acid sequence set forth in SEQ ID NO:44 and a VL that is at least 85% identical to the amino acid sequence set forth in SEQ ID NO:70. In another instance, the antibody that binds to both human αvβ1 and αvβ6 comprises a VH that is at least 90% identical to the amino acid sequence set forth in SEQ ID NO:44 and a VL that is at least 90% identical to the amino acid sequence set forth in SEQ ID NO:70. In yet another instance, the antibody that binds to both human αvβ1 and αvβ6 comprises a VH that is identical to the amino acid sequence set forth in SEQ ID NO:44 and a VL that is identical to the amino acid sequence set forth in SEQ ID NO:70.

In certain instances, an antibody that binds to both human αvβ1 and αvβ6 is one that competes with or binds to the same epitope as a reference antibody with a VH having the amino acid sequence set forth in SEQ ID NO:44 and a VL having the amino acid sequence set forth in SEQ ID NO:70.

### Exemplary Antibody 13

Exemplary Antibody 13 specifically binds to human αvβ1 and αvβ6 but not other integrins (e.g., other RGD family of integrins). The amino acid sequences of the CDRs and the mature heavy chain variable region and light chain variable regions of Exemplary Antibody 13 are provided below.

| | | Enhanced |
|---|---|---|
| Domain | Kabat | Chothia/AbM |
| VH CDR1 | DYYMS | FTFSDYYMS |
| | (SEQ ID NO: 36) | (SEQ ID NO: 37) |
| VH CDR2 | YISSSGSTIYYADSVKG | YISSSGSTIY |
| | (SEQ ID NO: 38) | (SEQ ID NO: 39) |
| VH CDR3 | GGPSRGDALAEYFQH | GGPSRGDALAEYFQH |
| | (SEQ ID NO: 46) | (SEQ ID NO: 46) |
| VL CDR1 | RASQSVSSNLA | RASQSVSSNLA |
| | (SEQ ID NO: 18) | (SEQ ID NO: 18) |
| VL CDR2 | GASTRAT | GASTRAT |
| | (SEQ ID NO: 47) | (SEQ ID NO: 47) |
| VL CDR3 | QQLTNHPPIA | QQLTNHPPIA |
| | (SEQ ID NO: 71) | (SEQ ID NO: 71) |

VH
(SEQ ID NO: 49)
QVQLVESGGGLVKPGGSLRLSCAASGFTFSDYYMSWIRQAPGK
GLEWVSYISSSGSTIYYADSVKGRFTISRDNAKNSLYLQMNSL
RAEDTAVYYCARGGPSRGDALAEYFQHWGQGTTVTVSS
VL
(SEQ ID NO: 72)
EIVMTQSPATLSVSPGERATLSCRASQSVSSNLAWYQQKPGQA
PRLLIYGASTRATGIPARFSGSGSGTEFTLTISSLQSEDFAVY
YCQQLTNHPPIAFGGGTKVEIK

In some instances, the antibody that binds to both human αvβ1 and αvβ6 comprises a VH comprising the three VH CDRs and a VL comprising the three VL CDRs of Exemplary Antibody 13. The six CDRs can be based on any definition known in the art such as, but not limited to, Kabat, Chothia, enhanced Chothia, contact, IMGT, or Honegger definitions.

In one instance, an antibody that binds to both human αvβ1 and αvβ6 comprises (i) a VH comprising a VHCDR1 comprising the amino acid sequence set forth in SEQ ID NO:37, a VHCDR2 comprising the amino acid sequence set forth in SEQ ID NO:39, and a VHCDR3 comprising the amino acid sequence set forth in SEQ ID NO:46; and (ii) a VL comprising a VLCDR1 comprising the amino acid sequence set forth in SEQ ID NO: 18, a VLCDR2 comprising the amino acid sequence set forth in SEQ ID NO:47, and a VLCDR3 comprising the amino acid sequence set forth in SEQ ID NO:71. In another instance, the antibody that binds to both human αvβ1 and αvβ6 comprises (i) a VH comprising a VHCDR1 comprising the amino acid sequence set forth in SEQ ID NO:36, a VHCDR2 comprising the amino acid sequence set forth in SEQ ID NO:38, and a VHCDR3 comprising the amino acid sequence set forth in SEQ ID NO: 46; and (ii) a VL comprising a VLCDR1 comprising the amino acid sequence set forth in SEQ ID NO:18, a VLCDR2 comprising the amino acid sequence set forth in SEQ ID NO:47, and a VLCDR3 comprising the amino acid sequence set forth in SEQ ID NO:71.

In some instances, an antibody that binds to both human αvβ1 and αvβ6 comprises a VH that is at least 75%, 80%, 85%, 90%, 91%, 92%, 93%, 94%, 95%, 96%, 97%, 98%, or 99% identical to the amino acid sequence set forth in SEQ ID NO:49. In some instances, the antibody that binds to both human αvβ1 and αvβ6 comprises a VL that is at least 75%, 80%, 85%, 90%, 91%, 92%, 93%, 94%, 95%, 96%, 97%, 98%, or 99% identical to the amino acid sequence set forth in SEQ ID NO:72. In one instance, the antibody that binds to both human αvβ1 and αvβ6 comprises a VH that is at least 85% identical to the amino acid sequence set forth in SEQ ID NO:49 and a VL that is at least 85% identical to the amino acid sequence set forth in SEQ ID NO:72. In another instance, the antibody that binds to both human αvβ1 and αvβ6 comprises a VH that is at least 90% identical to the amino acid sequence set forth in SEQ ID NO:49 and a VL that is at least 90% identical to the amino acid sequence set forth in SEQ ID NO:72. In yet another instance, the antibody that binds to both human αvβ1 and αvβ6 comprises a VH that is identical to the amino acid sequence set forth in SEQ ID NO:49 and a VL that is identical to the amino acid sequence set forth in SEQ ID NO:72.

In certain instances, an antibody that binds to both human αvβ1 and αvβ6 is one that competes with or binds to the same epitope as a reference antibody with a VH having the amino acid sequence set forth in SEQ ID NO:49 and a VL having the amino acid sequence set forth in SEQ ID NO:72.

### Exemplary Antibody 14

Exemplary Antibody 14 specifically binds to human αvβ1 and αvβ6 but not other integrins (e.g., other RGD family of integrins). The amino acid sequences of the CDRs and the mature heavy chain variable region and light chain variable regions of Exemplary Antibody 14 are provided below.

| | | Enhanced |
|---|---|---|
| Domain | Kabat | Chothia/AbM |
| VH CDR1 | DYYMS | FTFSDYYMS |
| | (SEQ ID NO: 36) | (SEQ ID NO: 37) |
| VH CDR2 | YISSSGSTIYYADSVKG | YISSSGSTIY |
| | (SEQ ID NO: 38) | (SEQ ID NO: 39) |
| VH CDR3 | ERGNRGDTPRYYYMDV | ERGNRGDTPRYYYMDV |
| | (SEQ ID NO: 73) | (SEQ ID NO: 73) |
| VL CDR1 | RASQSISRYLN | RASQSISRYLN |
| | (SEQ ID NO: 74) | (SEQ ID NO: 74) |
| VL CDR2 | AASSLQS | AASSLQS |
| | (SEQ ID NO: 42) | (SEQ ID NO: 42) |
| VL CDR3 | QQSLVTPFT | QQSLVTPFT |
| | (SEQ ID NO: 75) | (SEQ ID NO: 75) |

VH
(SEQ ID NO: 76)
QVQLVESGGGLVKPGGSLRLSCAASGFTFSDYYMSWIRQAPGK
GLEWVSYISSSGSTIYYADSVKGRFTISRDNAKNSLYLQMNSL
RAEDTAVYYCARERGNRGDTPRYYYMDVWGKGTTVTVSS
VL
(SEQ ID NO: 77)
DIQMTQSPSSLSASVGDRVTITCRASQSISRYLNWYQQKPGKA
PKLLIYAASSLQSGVPSRFSGSGSGTDFTLTISSLQPEDFATY
YCQQSLVTPFTFGGGTKVEIK

In some instances, the antibody that binds to both human αvβ1 and αvβ6 comprises a VH comprising the three VH CDRs and a VL comprising the three VL CDRs of Exemplary Antibody 14. The six CDRs can be based on any definition known in the art such as, but not limited to, Kabat, Chothia, enhanced Chothia, contact, IMGT, or Honegger definitions.

In one instance, an antibody that binds to both human αvβ1 and αvβ6 comprises (i) a VH comprising a VHCDR1 comprising the amino acid sequence set forth in SEQ ID NO:37, a VHCDR2 comprising the amino acid sequence set forth in SEQ ID NO:39, and a VHCDR3 comprising the amino acid sequence set forth in SEQ ID NO:73; and (ii) a VL comprising a VLCDR1 comprising the amino acid sequence set forth in SEQ ID NO:74, a VLCDR2 comprising the amino acid sequence set forth in SEQ ID NO:42, and a VLCDR3 comprising the amino acid sequence set forth in SEQ ID NO:75. In another instance, the antibody that binds to both human αvβ1 and αvβ6 comprises (i) a VH comprising a VHCDR1 comprising the amino acid sequence set forth in SEQ ID NO:36, a VHCDR2 comprising the amino acid sequence set forth in SEQ ID NO:38, and a VHCDR3 comprising the amino acid sequence set forth in SEQ ID NO: 73; and (ii) a VL comprising a VLCDR1 comprising the amino acid sequence set forth in SEQ ID NO:74, a VLCDR2 comprising the amino acid sequence set forth in SEQ ID NO:42, and a VLCDR3 comprising the amino acid sequence set forth in SEQ ID NO:75.

In some instances, an antibody that binds to both human αvβ1 and αvβ6 comprises a VH that is at least 75%, 80%, 85%, 90%, 91%, 92%, 93%, 94%, 95%, 96%, 97%, 98%, or 99% identical to the amino acid sequence set forth in SEQ ID NO:76. In some instances, the antibody that binds to both human αvβ1 and αvβ6 comprises a VL that is at least 75%, 80%, 85%, 90%, 91%, 92%, 93%, 94%, 95%, 96%, 97%, 98%, or 99% identical to the amino acid sequence set forth in SEQ ID NO:77. In one instance, the antibody that binds to both human αvβ1 and αvβ6 comprises a VH that is at least 85% identical to the amino acid sequence set forth in SEQ ID NO:76 and a VL that is at least 85% identical to the amino acid sequence set forth in SEQ ID NO:77. In another instance, the antibody that binds to both human αvβ1 and αvβ6 comprises a VH that is at least 90% identical to the amino acid sequence set forth in SEQ ID NO:76 and a VL that is at least 90% identical to the amino acid sequence set forth in SEQ ID NO:77. In yet another instance, the antibody that binds to both human αvβ1 and αvβ6 comprises a VH that is identical to the amino acid sequence set forth in SEQ ID NO:76 and a VL that is identical to the amino acid sequence set forth in SEQ ID NO:77.

In certain instances, an antibody that binds to both human αvβ1 and αvβ6 is one that competes with or binds to the same epitope as a reference antibody with a VH having the amino acid sequence set forth in SEQ ID NO:76 and a VL having the amino acid sequence set forth in SEQ ID NO:77.

C. Group III (Antibodies that Bind to αvβ1 Integrin and One or More Integrins Selected from the Group Consisting of αvβ3, αvβ5, αvβ6, αvβ8, α5β1, α8β1, and αIIBβ3 Integrin)

This disclosure further features antibodies that bind to αvβ1 integrin and one or more integrins selected from the group consisting of αvβ3, αvβ5, αvβ6, αvβ8, α5β1, α8β1, and αIIBβ3. In some instances, the antibodies do not bind to integrins other than the RGD-binding integrins. Such antibodies include the sequences of Exemplary Antibodies 15-20, which bind with high affinity of KD≤20 nM (bivalent affinity) to human αvβ1, and with affinity of 100 nM (bivalent affinity) to other RGD binding integrins.

### Exemplary Antibody 15

Exemplary Antibody 15 specifically binds to human αvβ1 and at least one (e.g., one, two, three, four, five, six, or seven) other RGD family integrin (e.g., αvβ3, αvβ5, αvβ6, αvβ8, α5β1, α8β1, and αIIBβ3). This antibody shows cation-independent binding to its target. The amino acid sequences of the CDRs and the mature heavy chain variable region and light chain variable regions of Exemplary Antibody 15 are provided below.

| | | Enhanced |
|---|---|---|
| Domain | Kabat | Chothia/AbM |
| VH CDR1 | SYYMH | YTFTSYYMH |
| | (SEQ ID NO: 3) | (SEQ ID NO: 4) |
| VH CDR2 | IINPSGGSTSYAQKFQG | IINPSGGSTS |
| | (SEQ ID NO: 5) | (SEQ ID NO: 6) |
| VH CDR3 | DRSGIAGRRWVYYYGMDV | DRSGIAGRRWVYYYGMDV |
| | (SEQ ID NO: 78) | (SEQ ID NO: 78) |
| VL CDR1 | RASQSVSSYLA | RASQSVSSYLA |
| | (SEQ ID NO: 79) | (SEQ ID NO: 79) |
| VL CDR2 | DASNRAT | DASNRAT |
| | (SEQ ID NO: 80) | (SEQ ID NO: 80) |
| VL CDR3 | QQRSNLPYT | QQRSNLPYT |
| | (SEQ ID NO: 81) | (SEQ ID NO: 81) |

VH
(SEQ ID NO: 82)
QVQLVQSGAEVKKPGASVKVSCKASGYTFTSYYMHWVRQAPGQ
GLEWMGIINPSGGSTSYAQKFQGRVTMTRDTSTSTVYMELSSL
RSEDTAVYYCARDRSGIAGRRWVYYYGMDVWGQGTTVTVSS
VL
(SEQ ID NO: 83)
EIVLTQSPATLSLSPGERATLSCRASQSVSSYLAWYQQKPGQA
PRLLIYDASNRATGIPARFSGSGSGTDFTLTISSLEPEDFAVY
YCQQRSNLPYTFGGGTKVEIK

In some instances, an antibody of Group III (i.e., an antibody that that binds to αvβ1 integrin and one or more integrins selected from the group consisting of αvβ3, αvβ5, αvβ6, αvβ8, α5β1, α8β1, and αIIBβ3) comprises a VH comprising the three VH CDRs and a VL comprising the three VL CDRs of Exemplary Antibody 15. The six CDRs can be based on any definition known in the art such as, but not limited to, Kabat, Chothia, enhanced Chothia, contact, IMGT, or Honegger definitions.

In one instance, an antibody of Group III comprises (i) a VH comprising a VHCDR1 comprising the amino acid sequence set forth in SEQ ID NO:4, a VHCDR2 comprising the amino acid sequence set forth in SEQ ID NO: 6, and a VHCDR3 comprising the amino acid sequence set forth in SEQ ID NO: 78; and (ii) a VL comprising a VLCDR1 comprising the amino acid sequence set forth in SEQ ID NO:79, a VLCDR2 comprising the amino acid sequence set forth in SEQ ID NO:80, and a VLCDR3 comprising the amino acid sequence set forth in SEQ ID NO:81. In another instance, an antibody of Group III comprises (i) a VH comprising a VHCDR1 comprising the amino acid sequence set forth in SEQ ID NO:3, a VHCDR2 comprising the amino acid sequence set forth in SEQ ID NO:5, and a VHCDR3 comprising the amino acid sequence set forth in SEQ ID NO:78; and (ii) a VL comprising a VLCDR1 comprising the amino acid sequence set forth in SEQ ID NO:79, a VLCDR2 comprising the amino acid sequence set forth in SEQ ID NO: 80, and a VLCDR3 comprising the amino acid sequence set forth in SEQ ID NO:81.

In some instances, an antibody of Group III comprises a VH that is at least 75%, 80%, 85%, 90%, 91%, 92%, 93%, 94%, 95%, 96%, 97%, 98%, or 99% identical to the amino acid sequence set forth in SEQ ID NO:82. In some instances, an antibody of Group III comprises a VL that is at least 75%, 80%, 85%, 90%, 91%, 92%, 93%, 94%, 95%, 96%, 97%, 98%, or 99% identical to the amino acid sequence set forth in SEQ ID NO:83. In one instance, an antibody of Group III comprises a VH that is at least 85% identical to the amino acid sequence set forth in SEQ ID NO:82 and a VL that is at least 85% identical to the amino acid sequence set forth in SEQ ID NO:83. In another instance, an antibody of Group III comprises a VH that is at least 90% identical to the amino acid sequence set forth in SEQ ID NO:82 and a VL that is at least 90% identical to the amino acid sequence set forth in SEQ ID NO:83. In yet another instance, an antibody of Group III comprises a VH that is identical to the amino acid sequence set forth in SEQ ID NO:82 and a VL that is identical to the amino acid sequence set forth in SEQ ID NO:83.

In certain instances, an antibody of Group III is one that competes with or binds to the same epitope as a reference antibody with a VH having the amino acid sequence set forth in SEQ ID NO:82 and a VL having the amino acid sequence set forth in SEQ ID NO:83.

### Exemplary Antibody 16

Exemplary Antibody 16 specifically binds to human αvβ1 and at least one (e.g., one, two, three, four, five, six, or seven) other RGD family integrin (e.g., αvβ3, αvβ5, αvβ6, αvβ8, α5β1, α8β1, and αIIBβ3). The amino acid sequences of the CDRs and the mature heavy chain variable region and light chain variable regions of Exemplary Antibody 16 are provided below.

| | | Enhanced |
|---|---|---|
| Domain | Kabat | Chothia/AbM |
| VH CDR1 | SYWIG | YSFTSYWIG |
| | (SEQ ID NO: 84) | (SEQ ID NO: 85) |
| VH CDR2 | IIYPGDSDTRYSPSFQG | IIYPGDSDTR |
| | (SEQ ID NO: 86) | (SEQ ID NO: 87) |
| VH CDR3 | GPRSRGDGPSNYYYMDV | GPRSRGDGPSNYYYMDV |
| | (SEQ ID NO: 88) | (SEQ ID NO: 88) |
| VL CDR1 | RASQSISSWLA | RASQSISSWLA |
| | (SEQ ID NO: 89) | (SEQ ID NO: 89) |
| VL CDR2 | KASSLES | KASSLES |
| | (SEQ ID NO: 90) | (SEQ ID NO: 90) |
| VL CDR3 | QQYHSFSFT | QQYHSFSFT |
| | (SEQ ID NO: 91) | (SEQ ID NO: 91) |

VH
(SEQ ID NO: 92)
EVQLVQSGAEVKKPGESLKISCKGSGYSFTSYWIGWVRQMPGK
GLEWMGITYPGDSDTRYSPSFQGQVTISADKSISTAYLQWSSL
KASDTAMYYCARGPRSRGDGPSNYYYMDVWGQGTLVTVSS
VL
(SEQ ID NO: 93)
DIQMTQSPSTLSASVGDRVTITCRASQSISSWLAWYQQKPGKA
PKLLIYKASSLESGVPSRFSGSGSGTEFTLTISSLQPDDFATY
YCQQYHSFSFTFGGGTKVEIK

In some instances, an antibody of Group III (i.e., an antibody that binds to αvβ1 integrin and one or more integrins selected from the group consisting of αvβ3, αvβ5, αvβ6, αvβ8, α5β1, α8β1, and αIIBβ3) comprises a VH comprising the three VH CDRs and a VL comprising the three VL CDRs of Exemplary Antibody 16. The six CDRs can be based on any definition known in the art such as, but not limited to, Kabat, Chothia, enhanced Chothia, contact, IMGT, or Honegger definitions.

In one instance, an antibody of Group III comprises (i) a VH comprising a VHCDR1 comprising the amino acid sequence set forth in SEQ ID NO:85, a VHCDR2 comprising the amino acid sequence set forth in SEQ ID NO:87, and a VHCDR3 comprising the amino acid sequence set forth in SEQ ID NO:88; and (ii) a VL comprising a VLCDR1 comprising the amino acid sequence set forth in SEQ ID NO:89, a VLCDR2 comprising the amino acid sequence set forth in SEQ ID NO:90, and a VLCDR3 comprising the amino acid sequence set forth in SEQ ID NO:91. In another instance, an antibody of Group III comprises (i) a VH comprising a VHCDR1 comprising the amino acid sequence set forth in SEQ ID NO:84, a VHCDR2 comprising the amino acid sequence set forth in SEQ ID NO:86, and a VHCDR3 comprising the amino acid sequence set forth in SEQ ID NO: 88; and (ii) a VL comprising a VLCDR1 comprising the amino acid sequence set forth in SEQ ID NO: 89, a VLCDR2 comprising the amino acid sequence set forth in SEQ ID NO: 90, and a VLCDR3 comprising the amino acid sequence set forth in SEQ ID NO:91.

In some instances, an antibody of Group III comprises a VH that is at least 75%, 80%, 85%, 90%, 91%, 92%, 93%, 94%, 95%, 96%, 97%, 98%, or 99% identical to the amino acid sequence set forth in SEQ ID NO:92. In some instances, an antibody of Group III comprises a VL that is at least 75%, 80%, 85%, 90%, 91%, 92%, 93%, 94%, 95%, 96%, 97%, 98%, or 99% identical to the amino acid sequence set forth in SEQ ID NO:93. In one instance, an antibody of Group III comprises a VH that is at least 85% identical to the amino acid sequence set forth in SEQ ID NO:92 and a VL that is at least 85% identical to the amino acid sequence set forth in SEQ ID NO:93. In another instance, an antibody of Group III comprises a VH that is at least 90% identical to the amino acid sequence set forth in SEQ ID NO:92 and a VL that is at least 90% identical to the amino acid sequence set forth in SEQ ID NO:93. In yet another instance, an antibody of Group III comprises a VH that is identical to the amino acid sequence set forth in SEQ ID NO:92 and a VL that is identical to the amino acid sequence set forth in SEQ ID NO:93.

In certain instances, an antibody of Group III is one that competes with or binds to the same epitope as a reference antibody with a VH having the amino acid sequence set forth in SEQ ID NO:92 and a VL having the amino acid sequence set forth in SEQ ID NO:93.

### Exemplary Antibody 17

Exemplary Antibody 17 specifically binds to human αvβ1 and at least one (e.g., one, two, three, four, five, six, or seven) other RGD family integrin (e.g., αvβ3, αvβ5, αvβ6, αvβ8, α5β1, α8β1, and αIIBβ3). Exemplary Antibody 17 also binds to cynomolgus monkey, mouse, and rat αvβ1. Exemplary Antibody 17 is internalized. In some instances, this antibody specifically binds to αvβ1 and αvβ3. The amino acid sequences of the CDRs and the mature heavy chain variable region and light chain variable regions of Exemplary Antibody 17 are provided below.

| | | Enhanced |
|---|---|---|
| Domain | Kabat | Chothia/AbM |
| VH CDR1 | SYWIG | YSFTSYWIG |
| | (SEQ ID NO: 84) | (SEQ ID NO: 85) |
| VH CDR2 | IIYPGDSDTRYSPSFQG | IIYPGDSDTR |
| | (SEQ ID NO: 86) | (SEQ ID NO: 87) |
| VH CDR3 | GPRSRGDGPSNYYYMDV | GPRSRGDGPSNYYYMDV |
| | (SEQ ID NO: 88) | (SEQ ID NO: 88) |
| VL CDR1 | RASQSISSWLA | RASQSISSWLA |
| | (SEQ ID NO: 89) | (SEQ ID NO: 89) |
| VL CDR2 | KASSLES | KASSLES |
| | (SEQ ID NO: 90) | (SEQ ID NO: 90) |
| VL CDR3 | QQYRPLPPT | QQYRPLPPT |
| | (SEQ ID NO: 94) | (SEQ ID NO: 94) |

VH
(SEQ ID NO: 92)
EVQLVQSGAEVKKPGESLKISCKGSGYSFTSYWIGWVRQMPGK
GLEWMGITYPGDSDTRYSPSFQGQVTISADKSISTAYLQWSSL
KASDTAMYYCARGPRSRGDGPSNYYYMDVWGQGTLVTVSS
VL
(SEQ ID NO: 95)
DIQMTQSPSTLSASVGDRVTITCRASQSISSWLAWYQQKPGKA
PKLLIYKASSLESGVPSRFSGSGSGTEFTLTISSLQPDDFATY
YCQQYRPLPPTFGGGTKVEIK

In some instances, an antibody of Group III (i.e., an antibody that binds to αvβ1 integrin and one or more integrins selected from the group consisting of αvβ3, αvβ5, αvβ6, αvβ8, α5β1, α8β1, and αIIBβ3) comprises a VH comprising the three VH CDRs and a VL comprising the three VL CDRs of Exemplary Antibody 17. The six CDRs can be based on any definition known in the art such as, but not limited to, Kabat, Chothia, enhanced Chothia, contact, IMGT, or Honegger definitions.

In one instance, an antibody of Group III comprises (i) a VH comprising a VHCDR1 comprising the amino acid sequence set forth in SEQ ID NO:85, a VHCDR2 comprising the amino acid sequence set forth in SEQ ID NO:87, and a VHCDR3 comprising the amino acid sequence set forth in SEQ ID NO:88; and (ii) a VL comprising a VLCDR1 comprising the amino acid sequence set forth in SEQ ID NO:89, a VLCDR2 comprising the amino acid sequence set forth in SEQ ID NO:90, and a VLCDR3 comprising the amino acid sequence set forth in SEQ ID NO:94. In another instance, an antibody of Group III comprises (i) a VH comprising a VHCDR1 comprising the amino acid sequence set forth in SEQ ID NO:84, a VHCDR2 comprising the amino acid sequence set forth in SEQ ID NO:86, and a VHCDR3 comprising the amino acid sequence set forth in SEQ ID NO:88; and (ii) a VL comprising a VLCDR1 comprising the amino acid sequence set forth in SEQ ID NO: 89, a VLCDR2 comprising the amino acid sequence set forth in SEQ ID NO: 90, and a VLCDR3 comprising the amino acid sequence set forth in SEQ ID NO:94.

In some instances, an antibody of Group III comprises a VH that is at least 75%, 80%, 85%, 90%, 91%, 92%, 93%, 94%, 95%, 96%, 97%, 98%, or 99% identical to the amino acid sequence set forth in SEQ ID NO:92. In some instances, an antibody of Group III comprises a VL that is at least 75%, 80%, 85%, 90%, 91%, 92%, 93%, 94%, 95%, 96%, 97%, 98%, or 99% identical to the amino acid sequence set forth in SEQ ID NO:95. In one instance, an antibody of Group III comprises a VH that is at least 85% identical to the amino acid sequence set forth in SEQ ID NO:92 and a VL that is at least 85% identical to the amino acid sequence set forth in SEQ ID NO:95. In another instance, an antibody of Group III comprises a VH that is at least 90% identical to the amino acid sequence set forth in SEQ ID NO:92 and a VL that is at least 90% identical to the amino acid sequence set forth in SEQ ID NO:95. In yet another instance, an antibody of Group III comprises a VH that is identical to the amino acid sequence set forth in SEQ ID NO:92 and a VL that is identical to the amino acid sequence set forth in SEQ ID NO:95.

In certain instances, an antibody of Group III is one that competes with or binds to the same epitope as a reference antibody with a VH having the amino acid sequence set forth in SEQ ID NO:92 and a VL having the amino acid sequence set forth in SEQ ID NO:95.

### Exemplary Antibody 18

Exemplary Antibody 18 specifically binds to human αvβ1 and at least one (e.g., one, two, three, four, five, six, or seven) other RGD family integrin (e.g., αvβ3, αvβ5, αvβ6, αvβ8, α5β1, α8β1, and αIIBβ3). In some instances, this antibody specifically binds to αvβ1, αvβ3, αvβ5, αvβ6, and αvβ8. This antibody shows cation-independent binding to its target. The amino acid sequences of the CDRs and the mature heavy chain variable region and light chain variable regions of Exemplary Antibody 18 are provided below.

| | | Enhanced |
|---|---|---|
| Domain | Kabat | Chothia/AbM |
| VH CDR1 | SFYMH | YTFRSFYMH |
| | (SEQ ID NO: 96) | (SEQ ID NO: 97) |
| VH CDR2 | VINPSLGSTGYAQKFQG | VINPSLGSTG |
| | (SEQ ID NO: 98) | (SEQ ID NO: 99) |
| VH CDR3 | ETNYYRGGPAFDI | ETNYYRGGPAFDI |
| | (SEQ ID NO: 23) | (SEQ ID NO: 23) |
| VL CDR1 | RSSQSLLHSNGYNYLD | RSSQSLLHSNGYNYLD |
| | (SEQ ID NO: 24) | (SEQ ID NO: 24) |
| VL CDR2 | LGSNRAS | LGSNRAS |
| | (SEQ ID NO: 25) | (SEQ ID NO: 25) |
| VL CDR3 | MQVLGTPPWT | MQVLGTPPWT |
| | (SEQ ID NO: 26) | (SEQ ID NO: 26) |

VH
(SEQ ID NO: 100)
QVQLVQSGAEVKKPGASVKVSCKASGYTFRSFYMHWVRQAPGQ
GLEWMGVINPSLGSTGYAQKFQGRVTMTRDTSTSTVYMELSSL
RSEDTAVYYCARETNYYRGGPAFDIWGQGTMVTVSS
VL
(SEQ ID NO: 28)
DIVMTQSPLSLPVTPGEPASISCRSSQSLLHSNGYNYLDWYLQ
KPGQSPQLLIYLGSNRASGVPDRFSGSGSGTDFTLKISRVEAE
DVGVYYCMQVLGTPPWTFGGGTKVEIK

In some instances, an antibody of Group III (i.e., an antibody that binds to αvβ1 integrin and one or more integrins selected from the group consisting of αvβ3, αvβ5, αvβ6, αvβ8, α5β1, α8β1, and αIIBβ3) comprises a VH comprising the three VH CDRs and a VL comprising the three VL CDRs of Exemplary Antibody 18. The six CDRs can be based on any definition known in the art such as, but not limited to, Kabat, Chothia, enhanced Chothia, contact, IMGT, or Honegger definitions.

In one instance, an antibody of Group III comprises (i) a VH comprising a VHCDR1 comprising the amino acid sequence set forth in SEQ ID NO:97, a VHCDR2 comprising the amino acid sequence set forth in SEQ ID NO:99, and a VHCDR3 comprising the amino acid sequence set forth in SEQ ID NO:23; and (ii) a VL comprising a VLCDR1 comprising the amino acid sequence set forth in SEQ ID NO:24, a VLCDR2 comprising the amino acid sequence set forth in SEQ ID NO:25, and a VLCDR3 comprising the amino acid sequence set forth in SEQ ID NO:26. In another instance, an antibody of Group III comprises (i) a VH comprising a VHCDR1 comprising the amino acid sequence set forth in SEQ ID NO:96, a VHCDR2 comprising the amino acid sequence set forth in SEQ ID NO:98, and a VHCDR3 comprising the amino acid sequence set forth in SEQ ID NO:23; and (ii) a VL comprising a VLCDR1 comprising the amino acid sequence set forth in SEQ ID NO:24, a VLCDR2 comprising the amino acid sequence set forth in SEQ ID NO:25, and a VLCDR3 comprising the amino acid sequence set forth in SEQ ID NO:26.

In some instances, an antibody of Group III comprises a VH that is at least 75%, 80%, 85%, 90%, 91%, 92%, 93%, 94%, 95%, 96%, 97%, 98%, or 99% identical to the amino acid sequence set forth in SEQ ID NO:100. In some instances, an antibody of Group III comprises a VL that is at least 75%, 80%, 85%, 90%, 91%, 92%, 93%, 94%, 95%, 96%, 97%, 98%, or 99% identical to the amino acid sequence set forth in SEQ ID NO:28. In one instance, an antibody of Group III comprises a VH that is at least 85% identical to the amino acid sequence set forth in SEQ ID NO:100 and a VL that is at least 85% identical to the amino acid sequence set forth in SEQ ID NO:28. In another instance, an antibody of Group III comprises a VH that is at least 90% identical to the amino acid sequence set forth in SEQ ID NO:100 and a VL that is at least 90% identical to the amino acid sequence set forth in SEQ ID NO:28. In yet another instance, an antibody of Group III comprises a VH that is identical to the amino acid sequence set forth in SEQ ID NO:100 and a VL that is identical to the amino acid sequence set forth in SEQ ID NO:28.

In certain instances, an antibody of Group III is one that competes with or binds to the same epitope as a reference antibody with a VH having the amino acid sequence set forth in SEQ ID NO:100 and a VL having the amino acid sequence set forth in SEQ ID NO:28.

### Exemplary Antibody 19

Exemplary Antibody 19 specifically binds to human αvβ1 and at least one (e.g., one, two, three, four, five, six, or seven) other RGD family integrin (e.g., αvβ3, αvβ5, αvβ6, αvβ8, α5β1, α8β1, and αIIBβ3). Exemplary Antibody 19 also binds to cynomolgus monkey, mouse, and rat αvβ1. Exemplary Antibody 19 is internalized. In some instances, this antibody specifically binds to αvβ1 and αvβ8. The amino acid sequences of the CDRs and the mature heavy chain variable region and light chain variable regions of Exemplary Antibody 19 are provided below.

| | | Enhanced |
|---|---|---|
| Domain | Kabat | Chothia/AbM |
| VH CDR1 | SYGMH | FTFSSYGMH |
| | (SEQ ID NO: 13) | (SEQ ID NO: 14) |
| VH CDR2 | VISYDGSNKYYADSVKG | VISYDGSNKY |
| | (SEQ ID NO: 15) | (SEQ ID NO: 16) |
| VH CDR3 | GGPTRGDGTRVYYYGMDV | GGPTRGDGTRVYYYGMDV |
| | (SEQ ID NO: 17) | (SEQ ID NO: 17) |
| VL CDR1 | KSSQSVLYSSNNKNYLA | KSSQSVLYSSNNKNYLA |
| | (SEQ ID NO: 101) | (SEQ ID NO: 101) |
| VL CDR2 | WASTRES | WASTRES |
| | (SEQ ID NO: 102) | (SEQ ID NO: 102) |
| VL CDR3 | QQYVAFPRT | QQYVAFPRT |
| | (SEQ ID NO: 103) | (SEQ ID NO: 103) |

VH
(SEQ ID NO: 21)
QVQLVESGGGVVQPGRSLRLSCAASGFTFSSYGMHWVRQAPGK
GLEWVAVISYDGSNKYYADSVKGRFTISRDNSKNTLYLQMNSL
RAEDTAVYYCARGGPTRGDGTRVYYYGMDVWGQGTTVTVSS
VL
(SEQ ID NO: 104)
DIVMTQSPDSLAVSLGERATINCKSSQSVLYSSNNKNYLAWYQ
QKPGQPPKLLIYWASTRESGVPDRFSGSGSGTDFTLTISSLQA
EDVAVYYCQQYVAFPRTFGGGTKVEIK

In some instances, an antibody of Group III (i.e., an antibody that binds to αvβ1 integrin and one or more integrins selected from the group consisting of αvβ3, αvβ5, αvβ6, αvβ8, α5β1, α8β1, and αIIBβ3) comprises a VH comprising the three VH CDRs and a VL comprising the three VL CDRs of Exemplary Antibody 19. The six CDRs can be based on any definition known in the art such as, but not limited to, Kabat, Chothia, enhanced Chothia, contact, IMGT, or Honegger definitions.

In one instance, an antibody of Group III comprises (i) a VH comprising a VHCDR1 comprising the amino acid sequence set forth in SEQ ID NO:14, a VHCDR2 comprising the amino acid sequence set forth in SEQ ID NO:16, and a VHCDR3 comprising the amino acid sequence set forth in SEQ ID NO:17; and (ii) a VL comprising a VLCDR1 comprising the amino acid sequence set forth in SEQ ID NO:101, a VLCDR2 comprising the amino acid sequence set forth in SEQ ID NO: 102, and a VLCDR3 comprising the amino acid sequence set forth in SEQ ID NO: 103. In another instance, an antibody of Group III comprises (i) a VH comprising a VHCDR1 comprising the amino acid sequence set forth in SEQ ID NO:13, a VHCDR2 comprising the amino acid sequence set forth in SEQ ID NO:15, and a VHCDR3 comprising the amino acid sequence set forth in SEQ ID NO:17; and (ii) a VL comprising a VLCDR1 comprising the amino acid sequence set forth in SEQ ID NO:101, a VLCDR2 comprising the amino acid sequence set forth in SEQ ID NO: 102, and a VLCDR3 comprising the amino acid sequence set forth in SEQ ID NO:103.

In some instances, an antibody of Group III comprises a VH that is at least 75%, 80%, 85%, 90%, 91%, 92%, 93%, 94%, 95%, 96%, 97%, 98%, or 99% identical to the amino acid sequence set forth in SEQ ID NO:21. In some instances, an antibody of Group III comprises a VL that is at least 75%, 80%, 85%, 90%, 91%, 92%, 93%, 94%, 95%, 96%, 97%, 98%, or 99% identical to the amino acid sequence set forth in SEQ ID NO:104. In one instance, an antibody of Group III comprises a VH that is at least 85% identical to the amino acid sequence set forth in SEQ ID NO:21 and a VL that is at least 85% identical to the amino acid sequence set forth in SEQ ID NO:104. In another instance, an antibody of Group III comprises a VH that is at least 90% identical to the amino acid sequence set forth in SEQ ID NO:21 and a VL that is at least 90% identical to the amino acid sequence set forth in SEQ ID NO:104. In yet another instance, an antibody of Group III comprises a VH that is identical to the amino acid sequence set forth in SEQ ID NO:21 and a VL that is identical to the amino acid sequence set forth in SEQ ID NO:104.

In certain instances, an antibody of Group III is one that competes with or binds to the same epitope as a reference antibody with a VH having the amino acid sequence set forth in SEQ ID NO:21 and a VL having the amino acid sequence set forth in SEQ ID NO:104.

### Exemplary Antibody 20

Exemplary Antibody 20 specifically binds to human αvβ1 and at least one (e.g., one, two, three, four, five, six, or seven) other RGD family integrin (e.g., αvβ3, αvβ5, αvβ6, αvβ8, α5β1, α8β1, and αIIBβ3). The amino acid sequences of the CDRs and the mature heavy chain variable region and light chain variable regions of Exemplary Antibody 20 are provided below.

| | | Enhanced |
|---|---|---|
| Domain | Kabat | Chothia/AbM |
| VH CDR1 | DYYMS | FTFSDYYMS |
| | (SEQ ID NO: 36) | (SEQ ID NO: 37) |
| VH CDR2 | YISSSGSTIYYADSVKG | YISSSGSTIY |
| | (SEQ ID NO: 38) | (SEQ ID NO: 39) |
| VH CDR3 | GGPSRGDALAEYFQH | GGPSRGDALAEYFQH |
| | (SEQ ID NO: 46) | (SEQ ID NO: 46) |
| VL CDR1 | RASQSVSRYLA | RASQSVSRYLA |
| | (SEQ ID NO: 105) | (SEQ ID NO: 105) |
| VL CDR2 | DASNRAT | DASNRAT |
| | (SEQ ID NO: 80) | (SEQ ID NO: 80) |
| VL CDR3 | QQLSLHPPYT | QQLSLHPPYT |
| | (SEQ ID NO: 106) | (SEQ ID NO: 106) |

VH
(SEQ ID NO: 49)
QVQLVESGGGLVKPGGSLRLSCAASGFTFSDYYMSWIRQAPGK
GLEWVSYISSSGSTIYYADSVKGRFTISRDNAKNSLYLQMNSL
RAEDTAVYYCARGGPSRGDALAEYFQHWGQGTTVTVSS
VL
(SEQ ID NO: 107)
EIVLTQSPATLSLSPGERATLSCRASQSVSRYLAWYQQKPGQA
PRLLIYDASNRATGIPARFSGSGSGTDFTLTISSLEPEDFAVY
YCQQLSLHPPYTFGGGTKVEIK

In some instances, an antibody of Group III (i.e., an antibody that binds to αvβ1 integrin and one or more integrins selected from the group consisting of αvβ3, αvβ5, αvβ6, αvβ8, α5β1, α8β1, and αIIBβ3) comprises a VH comprising the three VH CDRs and a VL comprising the three VL CDRs of Exemplary Antibody 20. The six CDRs can be based on any definition known in the art such as, but not limited to, Kabat, Chothia, enhanced Chothia, contact, IMGT, or Honegger definitions.

In one instance, an antibody of Group III comprises (i) a VH comprising a VHCDR1 comprising the amino acid sequence set forth in SEQ ID NO:37, a VHCDR2 comprising the amino acid sequence set forth in SEQ ID NO:39, and a VHCDR3 comprising the amino acid sequence set forth in SEQ ID NO:46; and (ii) a VL comprising a VLCDR1 comprising the amino acid sequence set forth in SEQ ID NO:105, a VLCDR2 comprising the amino acid sequence set forth in SEQ ID NO: 80, and a VLCDR3 comprising the amino acid sequence set forth in SEQ ID NO: 106. In another instance, an antibody of Group III comprises (i) a VH comprising a VHCDR1 comprising the amino acid sequence set forth in SEQ ID NO:36, a VHCDR2 comprising the amino acid sequence set forth in SEQ ID NO:38, and a VHCDR3 comprising the amino acid sequence set forth in SEQ ID NO:46; and (ii) a VL comprising a VLCDR1 comprising the amino acid sequence set forth in SEQ ID NO:105, a VLCDR2 comprising the amino acid sequence set forth in SEQ ID NO: 80, and a VLCDR3 comprising the amino acid sequence set forth in SEQ ID NO:106.

In some instances, an antibody of Group III comprises a VH that is at least 75%, 80%, 85%, 90%, 91%, 92%, 93%, 94%, 95%, 96%, 97%, 98%, or 99% identical to the amino acid sequence set forth in SEQ ID NO:49. In some instances, an antibody of Group III comprises a VL that is at least 75%, 80%, 85%, 90%, 91%, 92%, 93%, 94%, 95%, 96%, 97%, 98%, or 99% identical to the amino acid sequence set forth in SEQ ID NO:107. In one instance, an antibody of Group III comprises a VH that is at least 85% identical to the amino acid sequence set forth in SEQ ID NO:49 and a VL that is at least 85% identical to the amino acid sequence set forth in SEQ ID NO:107. In another instance, an antibody of Group III comprises a VH that is at least 90% identical to the amino acid sequence set forth in SEQ ID NO:49 and a VL that is at least 90% identical to the amino acid sequence set forth in SEQ ID NO: 107. In yet another instance, an antibody of Group III comprises a VH that is identical to the amino acid sequence set forth in SEQ ID NO:49 and a VL that is identical to the amino acid sequence set forth in SEQ ID NO:107.

In certain instances, an antibody of Group III is one that competes with or binds to the same epitope as a reference antibody with a VH having the amino acid sequence set forth in SEQ ID NO:49 and a VL having the amino acid sequence set forth in SEQ ID NO:107.

### Antibody Fragments

Antibody fragments (e.g., Fab, Fab′, F(ab′)₂, Facb, and Fv) may be prepared by proteolytic digestion of intact antibodies. For example, antibody fragments can be obtained by treating the whole antibody with an enzyme such as papain, pepsin, or plasmin. Papain digestion of whole antibodies produces F(ab)₂ or Fab fragments; pepsin digestion of whole antibodies yields F(ab′)₂ or Fab′; and plasmin digestion of whole antibodies yields Facb fragments.

Alternatively, antibody fragments can be produced recombinantly. For example, nucleic acids encoding the antibody fragments of interest can be constructed, introduced into an expression vector, and expressed in suitable host cells. See, e.g., Co, M. S. et al., J Immunol., 152:2968-2976 (1994); Better, M. and Horwitz, A. H., Methods in Enzymology, 178:476-496 (1989); Pluckthun, A. and Skerra, A., Methods in Enzymology, 178:476-496 (1989); Lamoyi, E., Methods in Enzymology, 121:652-663 (1989); Rousseaux, J. et al., Methods in Enzymology, (1989) 121:663-669 (1989); and Bird, R. E. et al., TIBTECH, 9:132-137 (1991)). Antibody fragments can be expressed in and secreted from E. coli, thus allowing the facile production of large amounts of these fragments. Antibody fragments can be isolated from the antibody phage libraries. Alternatively, Fab′-SH fragments can be directly recovered from E. coli and chemically coupled to form F(ab)₂ fragments (Carter et al., Bio/Technology, 10:163-167 (1992)). According to another approach, F(ab′)₂ fragments can be isolated directly from recombinant host cell culture. Fab and F(ab′)₂ fragment with increased in vivo half-life comprising a salvage receptor binding epitope residues are described in U.S. Pat. No. 5,869,046.

### Minibodies

Minibodies of any of the antibodies described herein include diabodies, single chain (scFv), and single-chain (Fv)₂ (sc(Fv)₂). In some instances, the minibody is fused to a human Fc or a CH3 domain of a human Fc. For example a scFv or sc(Fv)₂ that binds αvβ1 or αvβ1 and αvβ6 may be fused to a human IgG1 Fc or a human IgG1 CH3 domain. These domains may be modified to reduce effector function. These domains may be modified to reduce or prevent post-translational modifications (e.g., glycosylation).

A “diabody” is a bivalent minibody constructed by gene fusion (see, e.g., Holliger, P. et al., Proc. Natl. Acad. Sci. U.S.A, 90:6444-6448 (1993); EP 404,097; WO 93/11161). Diabodies are dimers composed of two polypeptide chains. The VL and VH domain of each polypeptide chain of the diabody are bound by linkers. The number of amino acid residues that constitute a linker can be between 2 to 12 residues (e.g., 3-10 residues or five or about five residues). The linkers of the polypeptides in a diabody are typically too short to allow the VL and VH to bind to each other. Thus, the VL and VH encoded in the same polypeptide chain cannot form a single-chain variable region fragment, but instead form a dimer with a different single-chain variable region fragment. As a result, a diabody has two antigen-binding sites.

An scFv is a single-chain polypeptide antibody obtained by linking the VH and VL with a linker (see e.g., Huston et al., Proc. Natl. Acad. Sci. U.S.A, 85:5879-5883 (1988); and Pluckthun, “The Pharmacology of Monoclonal Antibodies” Vol. 113, Ed Resenburg and Moore, Springer Verlag, New York, pp. 269-315, (1994)). The order of VHs and VLs to be linked is not particularly limited, and they may be arranged in any order. Examples of arrangements include: [VH] linker [VL]; or [VL] linker [VH]. The H chain V region and L chain V region in an scFv may be derived from any anti-integrin antibody described herein (e.g., Exemplary Antibody 1 to 20).

An sc(Fv)₂ is a minibody in which two VHs and two VLs are linked by a linker to form a single chain (Hudson, et al., J Immunol. Methods, (1999) 231: 177-189 (1999)). An sc(Fv)₂ can be prepared, for example, by connecting scFvs with a linker. The sc(Fv)₂ of the present invention include antibodies preferably in which two VHs and two VLs are arranged in the order of: VH, VL, VH, and VL ([VH] linker [VL] linker [VH] linker [VL]), beginning from the N terminus of a single-chain polypeptide; however the order of the two VHs and two VLs is not limited to the above arrangement, and they may be arranged in any order. Examples of arrangements are listed below:

[VL] linker [VH] linker [VH] linker [VL]

[VH] linker [VL] linker [VL] linker [VH]

[VH] linker [VH] linker [VL] linker [VL]

[VL] linker [VL] linker [VH] linker [VH]

[VL] linker [VH] linker [VL] linker [VH]

Normally, three linkers are required when four antibody variable regions are linked; the linkers used may be identical or different. There is no particular limitation on the linkers that link the VH and VL regions of the minibodies. In some embodiments, the linker is a peptide linker. Any arbitrary single-chain peptide comprising about three to 25 residues (e.g., 5, 6, 7, 8, 9, 10, 11, 12, 13, 14, 15, 16, 17, 18) can be used as a linker. Examples of such peptide linkers include: Ser; Gly Ser; Gly Gly Ser; Ser Gly Gly; Gly Gly Gly Ser (SEQ ID NO:108); Ser Gly Gly Gly (SEQ ID NO:109); Gly Gly Gly Gly Ser (SEQ ID NO:110); Ser Gly Gly Gly Gly (SEQ ID NO:111); Gly Gly Gly Gly Gly Ser (SEQ ID NO:112); Ser Gly Gly Gly Gly Gly (SEQ ID NO:113); Gly Gly Gly Gly Gly Gly Ser (SEQ ID NO:114); Ser Gly Gly Gly Gly Gly Gly (SEQ ID NO:115); (Gly Gly Gly Gly Ser)ₙ (SEQ ID NO:110)ₙ, wherein n is an integer of one or more; and (Ser Gly Gly Gly Gly)ₙ (SEQ ID NO:111)ₙ, wherein n is an integer of one or more.

In certain embodiments, the linker is a synthetic compound linker (chemical cross-linking agent). Examples of cross-linking agents that are available on the market include N-hydroxysuccinimide (NHS), disuccinimidylsuberate (DSS), bis(sulfosuccinimidyl)suberate (BS3), dithiobis(succinimidylpropionate) (DSP), dithiobis(sulfosuccinimidylpropionate) (DTSSP), ethyleneglycol bis(succinimidylsuccinate) (EGS), ethyleneglycol bis(sulfosuccinimidylsuccinate) (sulfo-EGS), disuccinimidyl tartrate (DST), disulfosuccinimidyl tartrate (sulfo-DST), bis[2-(succinimidooxycarbonyloxy)ethyl]sulfone (BSOCOES), and bis[2-(sulfosuccinimidooxycarbonyloxy)ethyl]sulfone (sulfo-BSOCOES).

The amino acid sequence of the VH or VL in the minibodies may include modifications such as substitutions, deletions, additions, and/or insertions. For example, the modification may be in one or more of the framework regions of the antibodies described herein (e.g., Exemplary Antibodies 1-20). In certain embodiments, the modification involves one, two, three, four, five, six, seven, eight, nine, ten, eleven, twelve, thirteen, fourteen, fifteen, sixteen, seventeen, eighteen, nineteen, or twenty amino acid substitutions in one or more framework regions of the VH and/or VL domain of the minibody. Such substitutions are made to improve the binding and/or functional activity of the minibody. In other embodiments, one, two, three, four, five, six, seven, eight, nine, ten, eleven, twelve, thirteen, fourteen, fifteen, sixteen, seventeen, eighteen, nineteen, or twenty amino acids of the FRs of the antibodies described herein may be deleted or added as long as there is αvβ1 (and binding to αvβ6, or binding to one or more of αvβ6, αvβ3, αvβ5, αvβ8, α5β1, α8β1, and αIIBβ3) binding and/or functional activity when VH and VL are associated.

### Bispecific or Multispecific Antibodies

Multispecific antibodies are antibodies that have binding specificities for two or more different epitopes. Bispecific antibodies are antibodies that have binding specificities for two different epitopes of one antigen, or two different antigens. Exemplary bispecific antibodies may bind to two different epitopes of the αvβ1 protein. Other such antibodies may combine an αvβ1 binding site with a binding site for another protein (e.g., αvβ6, αvβ3, αvβ5, αvβ8, α5β1, α8β1, αIIBβ3). In some instances, the bispecific antibody comprises a first VH and a first VL that specifically binds to αvβ1 and a second VH and a second VL that specifically binds to αvβ6. In other instances, the bispecific antibody comprises a first VH and a first VL that specifically binds to αvβ1 and a second VH and a second VL that specifically binds to both αvβ1 and αvβ6. In some instances, the bispecific antibody comprises a first VH and a first VL that specifically binds to αvβ1 and a second VH and a second VL that specifically binds to one or more of: αvβ6, αvβ3, αvβ5, αvβ8, α5β1, α81, and IIBβ3. In yet other instances, the bispecific antibody comprises a first VH and a first VL that specifically binds to both αvβ1 and αvβ6 and a second VH and a second VL that specifically binds to one or more of: αvβ6, αvβ3, αvβ5, αvβ8, α5β1, α81, and IIBβ3. Bispecific antibodies can be prepared as full length antibodies or low molecular weight forms thereof (e.g., F(ab′)₂ bispecific antibodies, scFv bispecific antibodies, sc(Fv)₂ bispecific antibodies, diabody bispecific antibodies).

Traditional production of full-length bispecific antibodies is based on the co-expression of two immunoglobulin heavy chain-light chain pairs, where the two chains have different specificities (Millstein et al., Nature, 305:537-539 (1983)). In a different approach, antibody variable domains with the desired binding specificities are fused to immunoglobulin constant domain sequences. DNAs encoding the immunoglobulin heavy chain fusions and, if desired, the immunoglobulin light chain, are inserted into separate expression vectors, and are co-transfected into a suitable host cell. This provides for greater flexibility in adjusting the proportions of the three polypeptide fragments. It is, however, possible to insert the coding sequences for two or all three polypeptide chains into a single expression vector when the expression of at least two polypeptide chains in equal ratios results in high yields.

According to another approach described in U.S. Pat. No. 5,731,168, the interface between a pair of antibody molecules can be engineered to maximize the percentage of heterodimers that are recovered from recombinant cell culture. The preferred interface comprises at least a part of the CH3 domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers.

Methods of making bispecific antibodies are well known in the art. See, e.g., Spasevska I, Duong M N, Klein C, Dumontet C (2015) Advances in Bispecific Antibodies Engineering: Novel Concepts for Immunotherapies. J Blood Disord Transfus 6:243. Doi:10.4172/2155-9864.1000243; and Husain, B. & Ellerman, D. BioDrugs (2018) 32: 441. doi.org/10.1007/s40259-018-0299-9.

Bispecific antibodies include cross-linked or “heteroconjugate” antibodies. For example, one of the antibodies in the heteroconjugate can be coupled to avidin, the other to biotin. Heteroconjugate antibodies may be made using any convenient cross-linking methods.

The “diabody” technology provides an alternative mechanism for making bispecific antibody fragments. The fragments comprise a VH connected to a VL by a linker which is too short to allow pairing between the two domains on the same chain. Accordingly, the VH and VL domains of one fragment are forced to pair with the complementary VL and VH domains of another fragment, thereby forming two antigen-binding sites.

### Multivalent Antibodies

A multivalent antibody may be internalized (and/or catabolized) faster than a bivalent antibody by a cell expressing an antigen to which the antibodies bind (e.g., αvβ1, αvβ1 and αvβ6). Any of the antibodies described herein can be multivalent antibodies with three or more antigen binding sites (e.g., tetravalent antibodies), which can be readily produced by recombinant expression of nucleic acid encoding the polypeptide chains of the antibody. The antibodies described herein can comprise a dimerization domain and three or more antigen binding sites. An exemplary dimerization domain comprises (or consists of) an Fc region or a hinge region. The antibodies described herein can comprise (or consist of) three to about eight (e.g., four) antigen binding sites. The multivalent antibody optionally comprises at least one polypeptide chain (e.g., at least two polypeptide chains), wherein the polypeptide chain(s) comprise two or more variable domains. For instance, the polypeptide chain(s) may comprise VD1-(X1)ₙ-VD2-(X2)ₙ-Fc, wherein VD1 is a first variable domain, VD2 is a second variable domain, Fc is a polypeptide chain of an Fc region, X1 and X2 represent an amino acid or peptide spacer, and n is 0 or 1.

### Conjugated Antibodies

The antibodies disclosed herein may be conjugated antibodies which are bound to various molecules including macromolecular substances such as polymers (e.g., polyethylene glycol (PEG), polyethylenimine (PEI) modified with PEG (PEI-PEG), polyglutamic acid (PGA) (N-(2-Hydroxypropyl) methacrylamide (HPMA) copolymers), hyaluronic acid, radioactive materials (e.g. ⁹⁰Y, ¹³¹I), fluorescent substances, luminescent substances, haptens, enzymes, metal chelates, and drugs.

In certain embodiments, the antibodies described herein are modified with a moiety that improves its stabilization and/or retention in circulation, e.g., in blood, serum, or other tissues, e.g., by at least 1.5, 2, 5, 10, 15, 20, 25, 30, 40, or 50-fold. For example, the antibodies described herein can be associated with (e.g., conjugated to) a polymer, e.g., a substantially non-antigenic polymer, such as a polyalkylene oxide or a polyethylene oxide. Suitable polymers will vary substantially by weight. Polymers having molecular number average weights ranging from about 200 to about 35,000 Daltons (or about 1,000 to about 15,000, and 2,000 to about 12,500) can be used. For example, the antibodies described herein can be conjugated to a water soluble polymer, e.g., a hydrophilic polyvinyl polymer, e.g., polyvinylalcohol or polyvinylpyrrolidone. Examples of such polymers include polyalkylene oxide homopolymers such as polyethylene glycol (PEG) or polypropylene glycols, polyoxyethylenated polyols, copolymers thereof and block copolymers thereof, provided that the water solubility of the block copolymers is maintained. Additional useful polymers include polyoxyalkylenes such as polyoxyethylene, polyoxypropylene, and block copolymers of polyoxyethylene and polyoxypropylene; polymethacrylates; carbomers; and branched or unbranched polysaccharides.

The above-described conjugated antibodies can be prepared by performing chemical modifications on the antibodies or the lower molecular weight forms thereof described herein. Methods for modifying antibodies are well known in the art (e.g., U.S. Pat. Nos. 5,057,313 and 5,156,840).

Antibodies with Reduced Effector Function

The interaction of antibodies and antibody-antigen complexes with cells of the immune system triggers a variety of responses, referred to herein as effector functions. Immune-mediated effector functions include two major mechanisms: antibody-dependent cell-mediated cytotoxicity (ADCC) and complement-dependent cytotoxicity (CDC). Both of them are mediated by the constant region of the immunoglobulin protein. The antibody Fc domain is, therefore, the portion that defines interactions with immune effector mechanisms.

IgG antibodies activate effector pathways of the immune system by binding to members of the family of cell surface Fcγ receptors and to C1q of the complement system. Ligation of effector proteins by clustered antibodies triggers a variety of responses, including release of inflammatory cytokines, regulation of antigen production, endocytosis, and cell killing. These responses can provoke unwanted side effects such as inflammation and the elimination of antigen-bearing cells.

Accordingly, the present invention further relates to anti-integrin binding proteins, including antibodies (e.g., anti-αvβ1, anti-αvβ1 and -αvβ6 antibodies), with reduced effector functions.

Effector function of an antibody of the present invention may be determined using one of many known assays. The antibody's effector function may be reduced relative to a second antibody. In some embodiments, where the antibody of interest has been modified to reduce effector function, the second antibody may be the unmodified or parental version of the antibody (such as Exemplary Antibody 1 to 20 with a wildtype Fc region (e.g., IgG1, IgG2, IgG3)).

Effector functions include ADCC, whereby antibodies bind Fc receptors on cytotoxic T cells, natural killer (NK) cells, or macrophages leading to cell death, and CDC, which is cell death induced via activation of the complement cascade (reviewed in Daeron, Annu. Rev. Immunol., 15:203-234 (1997); Ward and Ghetie, Therapeutic Immunol., 2:77-94 (1995); and Ravetch and Kinet, Annu. Rev. Immunol. 9:457-492 (1991)). Such effector functions generally require the Fc region to be combined with a binding domain (e.g. an antibody variable domain) and can be assessed using standard assays that are known in the art (see, e.g., WO 05/018572, WO 05/003175, and U.S. Pat. No. 6,242,195).

Effector functions can be avoided by using antibody fragments lacking the Fc domain such as Fab, Fab′2, or single chain Fv. An alternative is to use the IgG4 subtype antibody, which binds to FcγRI but which binds poorly to Cq and FcγRII and RIII. However, IgG4 antibodies may form aggregates since they have poor stability at low pH compared with IgG1 antibodies. The stability of an IgG4 antibody can be improved by substituting arginine at position 409 (according to the EU index proposed by Kabat et al., Sequences of proteins of immunological interest, 1991, 5^{th} ed.) with any one of: lysine, methionine, threonine, leucine, valine, glutamic acid, asparagine, phenylalanine, tryptophan, or tyrosine. Alternatively, and or in addition, the stability of an IgG4 antibody can be improved by substituting a CH3 domain of an IgG4 antibody with a CH3 domain of an IgG1 antibody, or by substituting the CH2 and CH3 domains of IgG4 with the CH2 and CH3 domains of IgG1. Accordingly, the anti-integrin antibodies of the present invention that are of IgG4 isotype can include modifications at position 409 and/or replacement of the CH2 and/or CH3 domains with the IgG1 domains so as to increase stability of the antibody while decreasing effector function. The IgG2 subtype also has reduced binding to Fc receptors, but retains significant binding to the H131 allotype of FcγRIIa and to C1q. Thus, additional changes in the Fc sequence may be required to eliminate binding to all the Fc receptors and to C1q.

Several antibody effector functions, including ADCC, are mediated by Fc receptors (FcRs), which bind the Fc region of an antibody. The affinity of an antibody for a particular FcR, and hence the effector activity mediated by the antibody, may be modulated by altering the amino acid sequence and/or post-translational modifications of the Fc and/or constant region of the antibody.

FcRs are defined by their specificity for immunoglobulin isotypes; Fc receptors for IgG antibodies are referred to as FcγR, for IgE as FceR, for IgA as FcuR and so on. Three subclasses of FcγR have been identified: FcγRI (CD64), FcγRII (CD32) and FcγRIII (CD16). Both FcγRII and FcγRIII have two types: FcγRIIa (CD32a) and FcγRIIB (CD32b); and FcγRIIIA (CD16a) and FcγRIIIB (CD16b). Because each FcγR subclass is encoded by two or three genes, and alternative RNA splicing leads to multiple transcripts, a broad diversity in FcγR isoforms exists. For example, FcγRII (CD32) includes the isoforms IIa, IIb1, IIb2 IIb3, and IIc.

The binding site on human and murine antibodies for FcγR has been previously mapped to the so-called “lower hinge region” consisting of residues G233-S239 (EU index numbering as in Kabat et al., Sequences of Proteins of Immunological Interest, 5th Ed. Public Health Service, National Institutes of Health, Bethesda, Md. (1991), Woof et al., Molec. Immunol. 23:319-330 (1986); Duncan et al., Nature 332:563 (1988); Canfield and Morrison, J Exp. Med. 173:1483-1491 (1991); Chappel et al., Proc. Natl. Acad. Sci USA 88:9036-9040 (1991)). Of residues G233-S239, P238 and S239 are among those cited as possibly being involved in binding. Other residues involved in binding to FcγR are: G316-K338 (Woof et al., Mol. Immunol., 23:319-330 (1986)); K274-R301 (Sarmay et al., Molec. Immunol. 21:43-51 (1984)); Y407-R416 (Gergely et al., Biochem. Soc. Trans. 12:739-743 (1984) and Shields et al., J Biol Chem 276: 6591-6604 (2001), Lazar G A et al., Proc Natl Acad Sci 103: 4005-4010 (2006)); N297; T299; E318; L234-S239; N265-E269; N297-T299; and A327-1332. These and other stretches or regions of amino acid residues involved in FcR binding may be evident to the skilled artisan from an examination of the crystal structures of Ig-FcR complexes (see, e.g., Sondermann et al. 2000 Nature 406(6793):267-73 and Sondermann et al. 2002 Biochem Soc Trans. 30(4):481-6). Accordingly, the anti-integrin antibodies of the present invention include modifications of one or more of the aforementioned residues to decrease effector function as needed.

Another approach for altering monoclonal antibody effector function include mutating amino acids on the surface of the monoclonal antibody that are involved in effector binding interactions (Lund, J., et al. (1991) J Immunol. 147(8): 2657-62; Shields, R. L. et al. (2001) J Biol. Chem. 276(9): 6591-604).

To reduce effector function, one can use combinations of different subtype sequence segments (e.g., IgG2 and IgG4 combinations) to give a greater reduction in binding to Fcγ receptors than either subtype alone (Armour et al., Eur. J. Immunol., 29:2613-1624 (1999); Mol. Immunol., 40:585-593 (2003)). A large number of Fc variants having altered and/or reduced affinities for some or all Fc receptor subtypes (and thus for effector functions) are known in the art. See, e.g., US 2007/0224188; US 2007/0148171; US 2007/0048300; US 2007/0041966; US 2007/0009523; US 2007/0036799; US 2006/0275283; US 2006/0235208; US 2006/0193856; US 2006/0160996; US 2006/0134105; US 2006/0024298; US 2005/0244403; US 2005/0233382; US 2005/0215768; US 2005/0118174; US 2005/0054832; US 2004/0228856; US 2004/132101; US 2003/158389; see also U.S. Pat. Nos. 7,183,387; 6,737,056; 6,538,124; 6,528,624; 6,194,551; 5,624,821; 5,648,260; and Wang, X., Mathieu, M. & Brezski, R. J., Protein Cell, (2018) 9: 63. doi.org/10.1007/s13238-017-0473-8. In certain embodiments amino acids at positions 232, 234, 235, 236, 237, 239, 264, 265, 267, 269, 270, 299, 325, 328, 329, and 330 (numbered according to EU numbering) are substituted to reduce effector function. Non-limiting examples of substitutions that reduce effector function include one or more of: K322A; L234A/L235A; G236T; G236R; G236Q; H268A; H268Q; V309L; A330S; P331S; V234A/G237A/P238S/H268A/V309L/A330S/P331S; E233P/L234V/L235A/G236Q+A327G/A330S/P331S; and L235E+E318A/K320A/K322A.

Antibodies of the present invention with reduced effector function include antibodies with reduced binding affinity for one or more Fc receptors (FcRs) relative to a parent or non-variant antibody. Accordingly, antibodies described herein with reduced FcR binding affinity includes antibodies that exhibit a 1.5-fold, 2-fold, 2.5-fold, 3-fold, 4-fold, 5-fold, 10-fold, 20-fold, or 25-fold or higher decrease in binding affinity to one or more Fc receptors compared to a parent or non-variant antibody. In some embodiments, an antibody of any of the antibodies described herein with reduced effector function binds to an FcR with about 10-fold less affinity relative to a parent or non-vanant antibody. In other embodiments, an antibody of any of the antibodies described herein with reduced effector function binds to an FcR with about 15-fold less affinity or with about 20-fold less affinity relative to a parent or non-variant antibody. The FcR receptor may be one or more of FcγRI (CD64), FcγRII (CD32), and FcγRIII, and isoforms thereof, and FcER, FcpR, Fc6R, and/or an FcuR. In particular embodiments, an antibody of any of the antibodies described herein with reduced effector function exhibits a 1.5-fold, 2-fold, 2.5-fold, 3-fold, 4-fold, or 5-fold or higher decrease in binding affinity to FcγRIIa.

In CDC, the antibody-antigen complex binds complement, resulting in the activation of the complement cascade and generation of the membrane attack complex. Activation of the classical complement pathway is initiated by the binding of the first component of the complement system (C1q) to antibodies (of the appropriate subclass) which are bound to their cognate antigen; thus, the activation of the complement cascade is regulated in part by the binding affinity of the immunoglobulin to C1q protein. To activate the complement cascade, it is necessary for C1q to bind to at least two molecules of IgG1, IgG2, or IgG3, but only one molecule of IgM, attached to the antigenic target (Ward and Ghetie, Therapeutic Immunology 2:77-94 (1995) p. 80). To assess complement activation, a CDC assay, e.g. as described in Gazzano-Santoro et al., J Immunol. Methods, 202:163 (1996), may be performed.

It has been proposed that various residues of the IgG molecule are involved in binding to C1q including the Glu318, Lys320 and Lys322 residues on the CH2 domain, amino acid residue 331 located on a turn in close proximity to the same beta strand, the Lys235 and Gly237 residues located in the lower hinge region, and residues 231 to 238 located in the N-terminal region of the CH2 domain (see e.g., Xu et al., J. Immunol. 150:152A (Abstract) (1993), WO94/29351; Tao et al, J. Exp. Med., 178:661-667 (1993); Brekke et al., Eur. J. Immunol., 24:2542-47 (1994); Burton et al; Nature, 288:338-344 (1980); Duncan and Winter, Nature 332:738-40 (1988); Idusogie et al J Immunol 164: 4178-4184 (2000; U.S. Pat. Nos. 5,648,260, and 5,624,821).

Antibodies described herein with reduced C1q binding can comprise an amino acid substitution at one, two, three, or four of amino acid positions 270, 322, 329 and 331 of the human IgG Fc region, where the numbering of the residues in the IgG Fc region is that of the EU index as in Kabat. As an example in IgG1, two mutations in the COOH terminal region of the CH2 domain of human IgG1—K322A and P329A—do not activate the CDC pathway and were shown to result in more than a 100 fold decrease in C1q binding (U.S. Pat. No. 6,242,195).

Accordingly, in certain embodiments, an antibody of the present invention exhibits reduced binding to a complement protein relative to a second antibody (such as Exemplary Antibody 1 to 20 with a wildtype Fc region (e.g., IgG1, IgG2, IgG3)). In certain embodiments, an antibody of the invention exhibits reduced binding to C1q by a factor of about 1.5-fold or more, about 2-fold or more, about 3-fold or more, about 4-fold or more, about 5-fold or more, about 6-fold or more, about 7-fold or more, about 8-fold or more, about 9-fold or more, about 10-fold or more, or about 15-fold or more, relative to the second antibody.

Thus, in certain embodiments of the invention, one or more of these residues may be modified, substituted, or removed or one or more amino acid residues may be inserted so as to decrease CDC activity of the antibodies provided herein.

In certain other embodiments, the present invention provides an antibody that exhibits reduced binding to one or more FcR receptors but that maintains its ability to bind complement (e.g., to a similar or, in some embodiments, to a lesser extent than a native, non-variant, or parent antibody). Accordingly, an antibody of the present invention may bind and activate complement while exhibiting reduced binding to an FcR, such as, for example, FcγRIIa (e.g., FcγRIIa expressed on platelets). Such an antibody with reduced or no binding to FcγRIIa (such as FcγRIIa expressed on platelets, for example) but that can bind Cq and activate the complement cascade to at least some degree will reduce the risk of thromboembolic events while maintaining perhaps desirable effector functions. In alternative embodiments, an antibody of the present invention exhibits reduced binding to one or more FcRs but maintains its ability to bind one or more other FcRs. See, for example, US 2007-0009523, 2006-0194290, 2005-0233382, 2004-0228856, and 2004-0191244, which describe various amino acid modifications that generate antibodies with reduced binding to FcRI, FcRII, and/or FcRIII, as well as amino acid substitutions that result in increased binding to one FcR but decreased binding to another FcR.

Accordingly, effector functions involving the constant region of an antibody described herein may be modulated by altering properties of the constant region, and the Fc region in particular. In certain embodiments, the antibody having decreased effector function is compared with a second antibody with effector function and which may be a non-variant, native, or parent antibody comprising a native constant or Fc region that mediates effector function. In some instances, if the antibody is an anti-integrin (e.g., anti-αvβ1 Ab; anti-αvβ1 and anti-αvβ6 Ab; anti-αvβ1 and another RGD-binding integrin Ab) human IgG1 antibody with a modified Fc that reduces effector function, the second antibody is a wild type human IgG1 antibody.

A native constant region comprises an amino acid sequence identical to the amino acid sequence of a constant chain region found in nature. Preferably, a control molecule used to assess relative effector function comprises the same type/subtype Fc region as does the test or variant antibody. A variant or altered Fc or constant region comprises an amino acid sequence which differs from that of a native sequence heavy chain region by virtue of at least one amino acid modification (such as, for example, post-translational modification, amino acid substitution, insertion, or deletion). Accordingly, the variant constant region may contain one or more amino acid substitutions, deletions, or insertions that results in altered post-translational modifications, including, for example, an altered glycosylation pattern. The variant constant region can have decreased effector function.

Antibodies with decreased effector function(s) may be generated by engineering or producing antibodies with variant constant, Fc, or heavy chain regions. Recombinant DNA technology and/or cell culture and expression conditions may be used to produce antibodies with altered function and/or activity. For example, recombinant DNA technology may be used to engineer one or more amino acid substitutions, deletions, or insertions in regions (such as, for example, Fc or constant regions) that affect antibody function including effector functions. Alternatively, changes in post-translational modifications, such as, e.g. glycosylation patterns, may be achieved by manipulating the host cell and cell culture and expression conditions by which the antibody is produced.

Certain embodiments of the present invention relate to an antibody comprising or consisting of the three heavy chain variable region and three light chain variable region CDR sequences (enhanced Chothia, Kabat, or any other CDR definition) from Exemplary Antibody 1 to 20, and further comprising an Fc region (e.g., the Fc region of IgG4) that confers reduced effector function compared to a native or parental Fc region.

Methods of generating any of the aforementioned antibody variants comprising amino acid substitutions are well known in the art. These methods include, but are not limited to, preparation by site-directed (or oligonucleotide-mediated) mutagenesis, PCR mutagenesis, and cassette mutagenesis of a prepared DNA molecule encoding the antibody or at least the constant region of the antibody. Site-directed mutagenesis is well known in the art (see, e.g., Carter et al., Nucleic Acids Res., 13:4431-4443 (1985) and Kunkel et al., Proc. Natl. Acad. Sci. USA, 82:488 (1987)). PCR mutagenesis is also suitable for making amino acid sequence variants of the starting polypeptide. See Higuchi, in PCR Protocols, pp. 177-183 (Academic Press, 1990); and Vallette et al., Nuc. Acids Res. 17:723-733 (1989). Another method for preparing sequence variants, cassette mutagenesis, is based on the technique described by Wells et al., Gene, 34:315-323 (1985).

Antibodies with Altered Glycosylation

Different glycoforms can profoundly affect the properties of a therapeutic, including pharmacokinetics, pharmacodynamics, receptor-interaction and tissue-specific targeting (Graddis et al., 2002, Curr Pharm Biotechnol. 3: 285-297). In particular, for antibodies, the oligosaccharide structure can affect properties relevant to protease resistance, the serum half-life of the antibody mediated by the FcRn receptor, phagocytosis and antibody feedback, in addition to effector functions of the antibody (e.g., binding to the complement complex C1, which induces CDC, and binding to FcγR receptors, which are responsible for modulating the ADCC pathway) (Nose and Wigzell, 1983; Leatherbarrow and Dwek, 1983; Leatherbarrow et al., 1985; Walker et al., 1989; Carter et al., 1992, PNAS, 89: 4285-4289).

Accordingly, another means of modulating effector function of antibodies includes altering glycosylation of the antibody constant region. Altered glycosylation includes, for example, a decrease or increase in the number of glycosylated residues, a change in the pattern or location of glycosylated residues, as well as a change in sugar structure(s). The oligosaccharides found on human IgGs affects their degree of effector function (Raju, T. S. BioProcess International April 2003. 44-53); the micro heterogeneity of human IgG oligosaccharides can affect biological functions such as CDC and ADCC, binding to various Fc receptors, and binding to C1q protein (Wright A. & Morrison S L. TIBTECH 1997, 15 26-32; Shields et al. J Biol Chem. 2001 276(9):6591-604; Shields et al. J Biol Chem. 2002; 277(30):26733-40; Shinkawa et al. J Biol Chem. 2003 278(5):3466-73; Umana et al. Nat Biotechnol. 1999 February; 17(2): 176-80). For example, the ability of IgG to bind C1q and activate the complement cascade may depend on the presence, absence or modification of the carbohydrate moiety positioned between the two CH2 domains (which is normally anchored at Asn297) (Ward and Ghetie, Therapeutic Immunology 2:77-94 (1995).

Glycosylation sites in an Fc-containing polypeptide, for example an antibody such as an IgG antibody, may be identified by standard techniques. The identification of the glycosylation site can be experimental or based on sequence analysis or modeling data. Consensus motifs, that is, the amino acid sequence recognized by various glycosyl transferases, have been described. For example, the consensus motif for an N-linked glycosylation motif is frequently NXT or NXS, where X can be any amino acid except proline. Several algorithms for locating a potential glycosylation motif have also been described. Accordingly, to identify potential glycosylation sites within an antibody or Fc-containing fragment, the sequence of the antibody is examined, for example, by using publicly available databases such as the website provided by the Center for Biological Sequence Analysis (see NetNGlyc services for predicting N-linked glycosylation sites and NetOGlyc services for predicting O-linked glycosylation sites).

In vivo studies have confirmed the reduction in the effector function of aglycosyl antibodies. For example, an aglycosyl anti-CD8 antibody is incapable of depleting CD8-bearing cells in mice (Isaacs, 1992 J. Immunol. 148: 3062) and an aglycosyl anti-CD3 antibody does not induce cytokine release syndrome in mice or humans (Boyd, 1995 supra; Friend, 1999 Transplantation 68:1632).

Importantly, while removal of the glycans in the CH2 domain appears to have a significant effect on effector function, other functional and physical properties of the antibody remains unaltered. Specifically, it has been shown that removal of the glycans had little to no effect on serum half-life and binding to antigen (Nose, 1983 supra; Tao, 1989 supra; Dorai, 1991 supra; Hand, 1992 supra; Hobbs, 1992 Mol. Immunol. 29:949).

The antibodies of the present invention may be modified or altered to elicit decreased effector function(s) compared to a second antibody (e.g., an Exemplary Antibody 1 to 20 with a wild type human IgG1, IgG2, IgG3 Fc region). Methods for altering glycosylation sites of antibodies are described, e.g., in U.S. Pat. Nos. 6,350,861 and 5,714,350, WO 05/18572 and WO 05/03175; these methods can be used to produce antibodies of the present invention with altered, reduced, or no glycosylation.

In some instances, the antibodies of this disclosure include an Fc region (e.g. a human IgG1 Fc) that has a modification that reduces or eliminates glycosylation in the Fc region (e.g., a T299A or N297Q substitution (numbering according to EU numbering)).

Alternatively, the antibodies of the present invention may be produced in a cell line which provides a desired glycosylation profile. For example, cells that make little afucosylated antibody, such as CHO cells, may be used for production. In another embodiment, manufacturing processes and/or media content or conditions may be manipulated to modulate the galactose and/or high mannose content. In one embodiment, the galactose/high mannose content of the antibody is low or reduced.

### Affinity Maturation

In one embodiment, an anti-integrin antibody described herein is modified, e.g., by mutagenesis, to provide a pool of modified antibodies. The modified antibodies are then evaluated to identify one or more antibodies having altered functional properties (e.g., improved binding, improved stability, reduced antigenicity, or increased stability in vivo). In one implementation, display library technology is used to select or screen the pool of modified antibodies. Higher affinity antibodies are then identified from the second library, e.g., by using higher stringency or more competitive binding and washing conditions. Other screening techniques can also be used.

In some implementations, the mutagenesis is targeted to regions known or likely to be at the binding interface. If, for example, the identified binding proteins are antibodies, then mutagenesis can be directed to the CDR regions of the heavy or light chains as described herein. Further, mutagenesis can be directed to framework regions near or adjacent to the CDRs, e.g., framework regions, particularly within 10, 5, or 3 amino acids of a CDR junction. In the case of antibodies, mutagenesis can also be limited to one or a few of the CDRs, e.g., to make step-wise improvements.

In one embodiment, mutagenesis is used to make an antibody more similar to one or more germline sequences. One exemplary germlining method can include: identifying one or more germline sequences that are similar (e.g., most similar in a particular database) to the sequence of the isolated antibody. Then mutations (at the amino acid level) can be made in the isolated antibody, either incrementally, in combination, or both. For example, a nucleic acid library that includes sequences encoding some or all possible germline mutations is made. The mutated antibodies are then evaluated, e.g., to identify an antibody that has one or more additional germline residues relative to the isolated antibody and that is still useful (e.g., has a functional activity). In one embodiment, as many germline residues are introduced into an isolated antibody as possible.

In one embodiment, mutagenesis is used to substitute or insert one or more germline residues into a CDR region. For example, the germline CDR residue can be from a germline sequence that is similar (e.g., most similar) to the variable region being modified. After mutagenesis, activity (e.g., binding or other functional activity) of the antibody can be evaluated to determine if the germline residue or residues are tolerated. Similar mutagenesis can be performed in the framework regions.

Selecting a germline sequence can be performed in different ways. For example, a germline sequence can be selected if it meets a predetermined criteria for selectivity or similarity, e.g., at least a certain percentage identity, e.g., at least 75, 80, 85, 90, 91, 92, 93, 94, 95, 96, 97, 98, 99, or 99.5% identity, relative to the donor nonhuman antibody. The selection can be performed using at least 2, 3, 5, or 10 germline sequences. In the case of CDR1 and CDR2, identifying a similar germline sequence can include selecting one such sequence. In the case of CDR3, identifying a similar germline sequence can include selecting one such sequence, but may include using two germline sequences that separately contribute to the amino-terminal portion and the carboxy-terminal portion. In other implementations, more than one or two germline sequences are used, e.g., to form a consensus sequence.

Calculations of “sequence identity” between two sequences are performed as follows. The sequences are aligned for optimal comparison purposes (e.g., gaps can be introduced in one or both of a first and a second amino acid or nucleic acid sequence for optimal alignment and non-homologous sequences can be disregarded for comparison purposes). The optimal alignment is determined as the best score using the GAP program in the GCG software package with a Blossum 62 scoring matrix with a gap penalty of 12, a gap extend penalty of 4, and a frameshift gap penalty of 5. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences.

In other embodiments, the antibody may be modified to have an altered glycosylation pattern (i.e., altered from the original or native glycosylation pattern). As used in this context, “altered” means having one or more carbohydrate moieties deleted, and/or having one or more glycosylation sites added to the original antibody. Addition of glycosylation sites to the presently disclosed antibodies may be accomplished by altering the amino acid sequence to contain glycosylation site consensus sequences; such techniques are well known in the art. Another means of increasing the number of carbohydrate moieties on the antibodies is by chemical or enzymatic coupling of glycosides to the amino acid residues of the antibody. These methods are described in, e.g., WO 87/05330, and Aplin and Wriston (1981) CRC Crit. Rev. Biochem., 22:259-306. Removal of any carbohydrate moieties present on the antibodies may be accomplished chemically or enzymatically as described in the art (Hakimuddin et al. (1987) Arch. Biochem. Biophys., 259:52; Edge et al. (1981) Anal. Biochem., 118:131; and Thotakura et al. (1987) Meth. Enzymol., 138:350). See, e.g., U.S. Pat. No. 5,869,046 for a modification that increases in vivo half life by providing a salvage receptor binding epitope.

Unlike in CDRs, more substantial changes in structure framework regions (FRs) can be made without adversely affecting the binding properties of an antibody. Changes to FRs include, but are not limited to, humanizing a nonhuman-derived framework or engineering certain framework residues that are important for antigen contact or for stabilizing the binding site, e.g., changing the class or subclass of the constant region, changing specific amino acid residues which might alter an effector function such as Fc receptor binding (Lund et al., J Immun., 147:2657-62 (1991); Morgan et al., Immunology, 86:319-24 (1995)), or changing the species from which the constant region is derived.

The anti-integrin antibodies can be in the form of full length (or whole) antibodies, or in the form of low molecular weight forms (e.g., biologically active antibody fragments or minibodies) of the anti-integrin antibodies, e.g., Fab, Fab′, F(ab′)₂, Fv, Fd, dAb, scFv, and sc(Fv)₂. Other anti-integrin antibodies encompassed by this disclosure include single domain antibody (sdAb) containing a single variable chain such as, VH or VL, or a biologically active fragment thereof. See, e.g., Moller et al., J. Biol. Chem., 285(49): 38348-38361 (2010); Harmsen et al., Appl. Microbiol. Biotechnol., 77(1):13-22 (2007); U.S. 2005/0079574 and Davies et al. (1996) Protein Eng., 9(6):531-7. Like a whole antibody, a sdAb is able to bind selectively to a specific antigen (e.g., αvβ1, αvβ1 and αvβ6). With a molecular weight of only 12-15 kDa, sdAbs are much smaller than common antibodies and even smaller than Fab fragments and single-chain variable fragments.

In certain embodiments, an anti-integrin antibody or antigen-binding fragment thereof or low molecular weight antibodies thereof specifically binds to αvβ1 or αvβ1 and αvβ6 reduces the severity of symptoms when administered to human patients having one or more of, or animal models of: fibrosis (e.g., liver fibrosis, lung fibrosis, kidney fibrosis), acute lung injury, acute kidney injury. In one embodiment, the anti-integrin antibody or low molecular weight antibodies thereof inhibit disease development in an idiopathic pulmonary fibrosis model (Degryse et al., Am J Med Sci., 341(6):444-9 (2011)). These features of an anti-integrin antibody or low molecular weight antibodies thereof can be measured according to methods known in the art.

### Nucleic Acids, Vector, Host Cells

This disclosure also features nucleic acids encoding the antibodies disclosed herein. Provided herein are nucleic acids encoding the VH CDR1, VH CDR2, and VH CDR3 of the anti-integrin antibodies described herein (e.g. Exemplary Antibodies 1-20). Also featured are nucleic acids encoding the VL CDR1, VL CDR2, and VL CDR3 of the anti-integrin antibodies described herein (e.g. Exemplary Antibodies 1-20). Provided herein are nucleic acids encoding the VH CDR1, VH CDR2, VH CDR3, VL CDR1, VL CDR2, and VL CDR3 of the anti-integrin antibodies described herein (e.g. Exemplary Antibodies 1-20). Also provided are nucleic acids encoding the heavy chain variable region (VH) of the anti-integrin antibodies described herein (e.g. Exemplary Antibodies 1-20), and/or nucleic acids encoding the light chain variable region (VL) of the anti-integrin antibodies described herein (e.g. Exemplary Antibodies 1-20). In certain instances, provided herein are nucleic acids encoding the VH and/or VL of the anti-integrin antibodies described herein (e.g. Exemplary Antibodies 1-20), linked to human heavy and/or human light chain constant regions, respectively. Also provided herein are nucleic acids encoding both VH and VL of the anti-integrin antibodies described herein (e.g. Exemplary Antibodies 1-20). In some instances, the nucleic acids described herein include a nucleic acid encoding the Fc region of a human antibody (e.g., human IgG1, IgG2, IgG3, or IgG4). In certain instances, the nucleic acids include a nucleic acid encoding the Fc region of a human antibody that has been modified to reduce or eliminate effector function (e.g., a N297Q or T299A substitution in a human IgG1 Fc region (numbering according to EU numbering)). In some cases, the nucleic acids include a nucleic acid encoding an Fc moiety that is a hIgG1 Fc, a hIgG2 Fc, a hIgG3 Fc, a hIgG4 Fc, a hIgG1agly Fc, a hIgG2 SAA Fc, a hIgG4(S228P) Fc, or a hIgG4(S228P)/G1 agly Fc.

Also disclosed herein are vectors (e.g. expression vectors) containing any of the nucleic acids described above.

Furthermore, this disclosure relates to host cells (e.g. bacterial cells, yeast cells, insect cells, or mammalian cells) containing the vector(s) or the nucleic acid(s) described above.

### Methods of Producing Anti-Integrin Antibodies

Antibodies, such as those described above, can be made, for example, by preparing and expressing synthetic genes that encode the recited amino acid sequences. Methods of generating variants (e.g., comprising amino acid substitutions) of any of the anti-integrin antibodies are well known in the art. These methods include, but are not limited to, preparation by site-directed (or oligonucleotide-mediated) mutagenesis, PCR mutagenesis, and cassette mutagenesis of a prepared DNA molecule encoding the antibody or any portion thereof (e.g., a framework region, a CDR, a constant region). Site-directed mutagenesis is well known in the art (see, e.g., Carter et al., Nucleic Acids Res., 13:4431-4443 (1985) and Kunkel et al., Proc. Natl. Acad. Sci. USA, 82:488 (1987)). PCR mutagenesis is also suitable for making amino acid sequence variants of the starting polypeptide. See Higuchi, in PCR Protocols, pp. 177-183 (Academic Press, 1990); and Vallette et al., Nuc. Acids Res. 17:723-733 (1989). Another method for preparing sequence variants, cassette mutagenesis, is based on the technique described by Wells et al., Gene, 34:315-323 (1985).

Antibodies or antigen binding fragments thereof may be produced in bacterial or eukaryotic cells. Some antibodies, e.g., Fab's, can be produced in bacterial cells, e.g., E. coli cells. Antibodies or antigen binding fragments thereof can also be produced in eukaryotic cells such as transformed cell lines (e.g., CHO, 293E, COS, Hela). In addition, antibodies (e.g., scFv's) can be expressed in a yeast cell such as Pichia (see, e.g., Powers et al., J Immunol Methods. 251:123-35 (2001)), Hanseula, or Saccharomyces. In one embodiment, the antibodies described herein are produced in the dihydrofolate reductase-deficient Chinese hamster ovary (CHO) cell line, DG44. In another embodiment, the antibodies described herein are produced in the DG44i cell line. To produce the antibody or antigen binding fragments thereof of interest, a polynucleotide encoding the antibody is constructed, introduced into an expression vector, and then expressed in suitable host cells. Standard molecular biology techniques are used to prepare the recombinant expression vector, transfect the host cells, select for transformants, culture the host cells and recover the antibody.

If the antibody is to be expressed in bacterial cells (e.g., E. coli), the expression vector should have characteristics that permit amplification of the vector in the bacterial cells. Additionally, when E. coli such as JM109, DH5α, HB101, or XL1-Blue is used as a host, the vector must have a promoter, for example, a lacZ promoter (Ward et al., 341:544-546 (1989), araB promoter (Better et al., Science, 240:1041-1043 (1988)), or T7 promoter that can allow efficient expression in E. coli. Examples of such vectors include, for example, M13-series vectors, pUC-series vectors, pBR322, pBluescript, pCR-Script, pGEX-5X-1 (Pharmacia), “QIAexpress system” (QIAGEN), pEGFP, and pET (when this expression vector is used, the host is preferably BL21 expressing T7 RNA polymerase). The expression vector may contain a signal sequence for antibody secretion. For production into the periplasm of E. coli, the pelB signal sequence (Lei et al., J Bacteriol., 169:4379 (1987)) may be used as the signal sequence for antibody secretion. For bacterial expression, calcium chloride methods or electroporation methods may be used to introduce the expression vector into the bacterial cell.

If the antibody is to be expressed in animal cells such as CHO, COS, and NIH3T3 cells, the expression vector includes a promoter necessary for expression in these cells, for example, an SV40 promoter (Mulligan et al., Nature, 277:108 (1979)), MMLV-LTR promoter, EF1α promoter (Mizushima et al., Nucleic Acids Res., 18:5322 (1990)), or CMV promoter. In addition to the nucleic acid sequence encoding the immunoglobulin or domain thereof, the recombinant expression vectors may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634,665 and 5,179,017). For example, typically the selectable marker gene confers resistance to drugs, such as G418, hygromycin, or methotrexate, on a host cell into which the vector has been introduced. Examples of vectors with selectable markers include pMAM, pDR2, pBK-RSV, pBK-CMV, pOPRSV, and pOP13.

In one embodiment, antibodies are produced in mammalian cells. Exemplary mammalian host cells for expressing an antibody include Chinese Hamster Ovary (CHO cells) (including dhfr⁻ CHO cells, described in Urlaub and Chasin (1980) Proc. Natl. Acad. Sci. USA 77:4216-4220, used with a DHFR selectable marker, e.g., as described in Kaufman and Sharp (1982) Mol. Biol. 159:601-621), human embryonic kidney 293 cells (e.g., 293, 293E, 293T), COS cells, NIH3T3 cells, lymphocytic cell lines, e.g., NS0 myeloma cells and SP2 cells, and a cell from a transgenic animal, e.g., a transgenic mammal. For example, the cell is a mammary epithelial cell.

In an exemplary system for antibody expression, recombinant expression vectors encoding the antibody heavy chain and the antibody light chain of any antibody described herein, respectively (e.g., Exemplary Antibody 1 to 20) are introduced into dhfr⁻ CHO cells by calcium phosphate-mediated transfection. In a specific embodiment, the dhfr− CHO cells are cells of the DG44 cell line, such as DG44i (see, e.g., Derouaz et al., Biochem Biophys Res Commun. 340(4):1069-77 (2006)). Within the recombinant expression vectors, the antibody heavy and light chain genes are each operatively linked to enhancer/promoter regulatory elements (e.g., derived from SV40, CMV, adenovirus and the like, such as a CMV enhancer/AdMLP promoter regulatory element or an SV40 enhancer/AdMLP promoter regulatory element) to drive high levels of transcription of the genes. The recombinant expression vectors also carry aDHFR gene, which allows for selection of CHO cells that have been transfected with the vector using methotrexate selection/amplification. The selected transformant host cells are cultured to allow for expression of the antibody heavy and light chains and the antibody is recovered from the culture medium.

Antibodies can also be produced by a transgenic animal. For example, U.S. Pat. No. 5,849,992 describes a method of expressing an antibody in the mammary gland of a transgenic mammal. A transgene is constructed that includes a milk-specific promoter and nucleic acids encoding the antibody of interest and a signal sequence for secretion. The milk produced by females of such transgenic mammals includes, secreted-therein, the antibody of interest. The antibody can be purified from the milk, or for some applications, used directly. Animals are also provided comprising one or more of the nucleic acids described herein.

The antibodies of the present disclosure can be isolated from inside or outside (such as medium) of the host cell and purified as substantially pure and homogenous antibodies. Methods for isolation and purification commonly used for antibody purification may be used for the isolation and purification of antibodies, and are not limited to any particular method. Antibodies may be isolated and purified by appropriately selecting and combining, for example, column chromatography, filtration, ultrafiltration, salting out, solvent precipitation, solvent extraction, distillation, immunoprecipitation, SDS-polyacrylamide gel electrophoresis, isoelectric focusing, dialysis, and recrystallization. Chromatography includes, for example, affinity chromatography, ion exchange chromatography, hydrophobic chromatography, gel filtration, reverse-phase chromatography, and adsorption chromatography (Strategies for Protein Purification and Characterization: A Laboratory Course Manual. Ed Daniel R. Marshak et al., Cold Spring Harbor Laboratory Press, 1996). Chromatography can be carried out using liquid phase chromatography such as HPLC and FPLC. Columns used for affinity chromatography include protein A column and protein G column. Examples of columns using protein A column include Hyper D, POROS, and Sepharose FF (GE Healthcare Biosciences). The present disclosure also includes antibodies that are highly purified using these purification methods.

### Characterization of the Antibodies

The integrin-binding properties of the antibodies described herein may be measured by any standard method, e.g., one or more of the following methods: OCTET®, Surface Plasmon Resonance (SPR), BIACORE™ analysis, Enzyme Linked Immunosorbent Assay (ELISA), EIA (enzyme immunoassay), RIA (radioimmunoassay), and Fluorescence Resonance Energy Transfer (FRET).

The binding interaction of a protein of interest (an anti-integrin antibody) and a target (e.g., an integrin) can be analyzed using the OCTET® systems. In this method, one of several variations of instruments (e.g., OCTET® QKe and QK), made by the ForteBio company are used to determine protein interactions, binding specificity, and epitope mapping. The OCTET® systems provide an easy way to monitor real-time binding by measuring the changes in polarized light that travels down a custom tip and then back to a sensor.

The binding interaction of a protein of interest (an anti-integrin antibody) and a target (e.g., an integrin) can be analyzed using Surface Plasmon Resonance (SPR). SPR or Biomolecular Interaction Analysis (BIA) detects bispecific interactions in real time, without labeling any of the interactants. Changes in the mass at the binding surface (indicative of a binding event) of the BIA chip result in alterations of the refractive index of light near the surface (the optical phenomenon of surface plasmon resonance (SPR)). The changes in the refractivity generate a detectable signal, which are measured as an indication of real-time reactions between biological molecules. Methods for using SPR are described, for example, in U.S. Pat. No. 5,641,640; Raether (1988) Surface Plasmons Springer Verlag; Sjolander and Urbaniczky (1991) Anal. Chem. 63:2338-2345; Szabo et al. (1995) Curr. Opin. Struct. Biol. 5:699-705 and on-line resources provide by BIAcore International AB (Uppsala, Sweden). Information from SPR can be used to provide an accurate and quantitative measure of the equilibrium dissociation constant (K_{d}), and kinetic parameters, including Kₒₙ and K_{off}, for the binding of a biomolecule to a target.

Epitopes can also be directly mapped by assessing the ability of different antibodies to compete with each other for binding to human αvβ1, αvβ6, or one or more RGD-binding integrins selected from the group consisting of αvβ3, αvβ5, αvβ8, α5β1, α8β1, and αIIBβ3 using BIACORE chromatographic techniques (Pharmacia BIAtechnology Handbook, “Epitope Mapping”, Section 6.3.2, (May 1994); see also Johne et al. (1993) J Immunol. Methods, 160:191-198).

When employing an enzyme immunoassay, a sample containing an antibody, for example, a culture supernatant of antibody-producing cells or a purified antibody is added to an antigen-coated plate. A secondary antibody labeled with an enzyme such as alkaline phosphatase is added, the plate is incubated, and after washing, an enzyme substrate such as p-nitrophenylphosphate is added, and the absorbance is measured to evaluate the antigen binding activity.

Additional general guidance for evaluating antibodies, e.g., Western blots and immunoprecipitation assays, can be found in Antibodies: A Laboratory Manual, ed. by Harlow and Lane, Cold Spring Harbor press (1988)).

### Indications

A. Group I-III Antibodies

αvβ1 integrin is highly expressed on activated fibroblasts, and plays a role in activating transforming growth factor β (TGFβ) and in driving tissue fibrosis (Reed et al., Sci Transl Med., 7:288 (2015)). Any of the antibodies described herein (e.g. Group I, II and III antibodies) can therefore be used in the treatment or prevention of any fibrotic diseases or conditions described herein or known in the art. In some embodiments, antibodies described herein are useful to treat or prevent such diseases or conditions at least because they block the activation of TGFβ.

The antibodies provided herein can be used to treat or prevent organ fibrosis, soft tissue fibrosis, joint and connective tissue fibrosis, and multi-organ or systemic fibrosis. Non-limiting examples of organ fibrosis include lung fibrosis, kidney fibrosis, liver/hepatic fibrosis, head and neck fibrosis, spinal cord injury/fibrosis, glial scarring in the brain, eye fibrosis, cardiac fibrosis, skin fibrosis, and bone marrow fibrosis. Non-limiting examples of soft tissue fibrosis include mediastinal fibrosis and retroperitoneal fibrosis. Non-limiting examples of joint and connective tissue fibrosis include arthrofibrosis and adhesive capsulitis. Non-limiting examples of multi-organ or systemic fibrosis include sarcoidosis, systemic sclerosis, amyloidosis, surgical fibrosis, and nephrogenic systemic fibrosis.

The antibodies provided herein can be used to treat or prevent lung fibrosis, such as, but not limited to IPF (idiopathic pulmonary fibrosis), acute exacerbations of IPF, radiation induced lung injury/fibrosis, flu induced fibrosis, coagulation induced fibrosis, vascular injury induced fibrosis, usual interstitial pneumonia (UIP), chronic obstructive pulmonary disease (COPD), bleomycin induced fibrosis, asthma (e.g., chronic asthma), silicosis, asbestos induced fibrosis, acute lung injury, and acute respiratory distress (including bacterial pneumonia induced, trauma induced, viral pneumonia induced, ventilator induced, non-pulmonary sepsis induced and aspiration induced), pulmonary histiocytosis X, and progressive massive fibrosis.

The antibodies provided herein can be used to treat or prevent kidney fibrosis, such as, but not limited to acute kidney injury, idiopathic nephrotic syndrome, idiopathic membranoproliferative glomerulonephritis, chronic nephropathies associated with injury and/or fibrosis (e.g. lupus, diabetes, scleroderma, glomerular nephritis, focal segmental glomerular sclerosis, IgA nephropathy, hypertension, allograft and Alport's disease).

The antibodies provided herein can be used to treat or prevent liver/hepatic fibrosis (such as, but not limited to acute liver injury, biliary duct injury induced fibrosis, and cirrhosis), intestinal fibrosis (such as, but not limited to, inflammatory bowel disease and Crohn's disease), eye fibrosis (such as, but not limited to corneal scarring, LASIX, corneal transplant, and trabeculectomy), cardiac fibrosis (such as, but not limited to idiopathic restrictive cardiomyopathy, atrial fibrosis, endomyocardial fibrosis, and myocardial infarction), skin fibrosis (such as, but not limited to hypertrophic scarring, burn induced fibrosis, psoriasis, and keloid), as well as bone marrow fibrosis (such as, but not limited to myelofibrosis).

The antibodies provided herein can be used to treat or prevent Nonalcoholic fatty liver disease (NAFLD) such as fatty liver disease and nonalcoholic steatohepatitis (NASH).

B. Group II Antibodies

The αvβ6 integrin can bind to several ligands including fibronectin, tenascin, and the latency associated peptide-1 and -3 (LAP1 and LAP3) (the N-terminal 278 amino acids of the latent precursor form of TGF-β1). The TGF-β cytokine is synthesized as a latent complex in which the N-terminal LAP is non-covalently associated with the mature active C-terminal TGF-β cytokine. The latent TGF-β complex cannot bind to its cognate receptor and thus is not biologically active until converted to an active form. αvβ6 binds LAP1 and LAP3 through interaction with an arginine-glycine-aspartate (“RGD”) motif and this binding of αvβ6 to LAP1 or LAP3 leads to activation of the latent precursor form of TGF-β1 and TGF-β3 as a result of a conformational change in the latent complex allowing TGF-β to bind to its receptor. Thus, upregulated expression of αvβ6 can lead to local activation of TGF-β, which in turn can activate a cascade of downstream events.

The TGF-β cytokine is a pleiotropic growth factor that regulates cell proliferation, differentiation, and immune responses. TGF-β also plays a role in cancer. TGF-β is recognized to have tumor suppressor and growth inhibitory activity, yet many tumors evolve a resistance to growth suppressive activities of TGF-β. In established tumors, TGF-β expression and activity has been implicated in promoting tumor survival, progression, and metastases. This is thought to be mediated by both autocrine and paracrine effects in the local tumor-stromal environment, including the effects of TGF-β on immune surveillance, angiogenesis, and increased tumor interstitial pressure. Several studies have shown the antitumor and anti-metastatic effects of inhibiting TGF-β.

The αvβ6 integrin is expressed on epithelial cells at relatively low levels in healthy tissue and significantly upregulated during development, injury, and wound healing. Expression of αvβ6 integrin is upregulated on cancers of epithelial origin, including colon cancer, squamous cell cancer, ovarian cancer, and breast cancer.

The antibodies described herein that bind to both αvβ1 and αvβ6 integrins but not to other integrins (i.e. Group II antibodies) can be used to protect against epithelial and/or endothelial cell injury (e.g. alveolar epithelial injury). Group II antibodies described herein can be used to block interaction of the αvβ6 receptor with RGD-containing ligands, e.g., proteins on the surface of viruses, thereby reducing or preventing viral infection.

Group II antibodies described herein can be used for treating cancer or cancer metastasis (including tumor growth and invasion), such as, but not limited to epithelial cancers. Non-limiting examples of epithelial cancers include squamous cell carcinoma, e.g., head and neck (including oral, laryngeal, pharyngeal, esophageal), breast, lung, prostate, cervical, colon, pancreatic, skin (basal cell carcinomas), ovarian and kidney cancers (e.g. renal cell cancer). Group II antibodies described herein can also be used for brain and central nervous system tumors (e.g. glioblastoma), ophthalmic diseases (e.g. macular degeneration and age-related macular degeneration), osteoporosis, as well as renal diseases, such as, but not limited to chronic tubular injury, chronic kidney disease, (chronic) interstitial fibrosis, tubular atrophy, and chronic allograft dysfunction in renal transplant patients.

The present disclosure includes methods of treating or preventing metastatic cancers by identifying pre-invasive lesions or carcinomas in patients, and treating the patient to eliminate the pre-invasive lesion before it has the opportunity to evolve into an invasive form. Such methods comprise, for example, (a) obtaining a tissue sample that is suspected of containing a cancer or a pre-invasive lesion, and a tissue sample that does not contain a cancer or pre-invasive lesion (preferably from the same tissue or organ as that suspected of containing a cancer or pre-invasive lesion); (b) contacting the tissue samples with one or more αvβ6-binding ligands, such as any Group II antibodies described herein, under conditions favoring the binding of the one or more αvβ6-binding ligands to αvβ6 integrins in the tissue wherever present; and (c) detecting the level or pattern of binding of the αvβ6-binding ligand(s) to the tissue, wherein an increase in the localized binding of the αvβ6-binding ligand in the myoepithelium surrounding a hyperplasia (e.g., a tumor) relative to the binding in the hyperplasia itself (or cells thereof), or an increase in the level of binding of the αvβ6-binding ligand in the tissue sample containing the cancerous or pre-invasive lesion relative to the binding in the non-cancerous tissue sample (or cells thereof), is indicative of carcinoma that is more likely to become invasive and potentially metastasize. In other related embodiments, the invention contemplates methods of reducing or preventing the progression of a pre-metastatic or pre-invasive tumor to a metastatic or invasive tumor in a patient, comprising administering to the patient a therapeutically effective amount of one or more ligands that bind to one or more subunits of integrin αvβ6 on one or more cells in the pre-metastatic or pre-invasive tumor, wherein the binding of the ligand to the integrin results in the reduction or prevention of invasion of cells of the pre-metastatic or pre-invasive cancer into tissue areas surrounding the primary tumor. In other embodiments, the methods of the invention are suitable for eliminating residual tumor cells, e.g., of residual metastatic cells, following removal, treatment or eradication of a tumor by a different approach. For example, such methods can be used to eliminate residual tumor cells or metastatic cells that may remain in the patient following surgical excision of a tumor, or tumor eradication by methods such as irradiation, chemotherapy and the like. In such therapeutic regimens, the methods of the invention may comprise administering the αvβ6-binding antibodies, to a patient prior to, during, and/or following surgical, radiological and/or chemotherapeutic ablation of the tumor.

C. Group III Antibodies

The antibodies described herein that bind to αvβ1 and one or more integrins selected from the group consisting of αvβ3, αvβ5, αvβ6, αvβ8, α5β1, α8β1, and IIBβ3 (i.e. Group III antibodies) can be used for treating cancer or cancer metastases, such as but not limited to, solid tumors (e.g. pancreatic cancer or breast cancer). Group III antibodies described herein can be used for treating ovarian, colorectal and prostate cancers, with or without bone metastases, and for treating renal cell cancer, peritoneal cancer, brain and central nervous system tumors, and melanoma. Group III antibodies described herein can be used for treating ophthalmic diseases, such as but not limited to, macular degeneration, age-related macular degeneration (AMD), wet age-related macular degeneration, diabetic macular edema, and diabetic retinopathy. Group III antibodies described herein can also be used for treating acute coronary syndrome (ACS), autoimmune diseases, and osteoporosis. Non limiting examples of autoimmune diseases include rheumatoid arthritis, psoriasis, lupus, inflammatory bowel disease, multiple sclerosis, Guillain-Barre syndrome, chronic inflammatory demyelinating polyneuropathy, Graves' disease, Hashimoto's thyroiditis, myasthenia gravis, and vasculitis. Group III antibodies described herein can be useful as an anti-thrombotic and can be used, for example, during percutaneous coronary intervention (angioplasty with or without stent placement).

The efficacy of the antibodies of the invention can be assessed in various animal models. Mouse models for lung fibrosis include bleomycin- (Pittet et al., J. Clin. Invest., 107(12):1537-1544 (2001); and Munger et al., Cell, 96:319-328 (1999)) and irradiation-inducible lung fibrosis (Franko et al., Rad. Res., 140:347-355 (1994)). Mouse models for kidney fibrosis include COL4A3−/− mice (see, e.g., Cosgrove et al., Amer. J. Path., 157:1649-1659 (2000), mice with adriamycin-induced injury (Wang et al., Kidney International, 58: 1797-1804 (2000); Deman et al., Nephrol Dial Transplant, 16: 147-150 (2001)), db/db mice (Ziyadeh et al., Proc. Natl. Acad. Sci. USA, 97:8015-8020 (2000)), and mice with unilateral ureteral obstruction (Fogo et al., Lab Investigation, 81: 189A (2001); and Fogo et al., Journal of the American Society of Nephrology, 12:819 A (2001)). αvβ6 antibodies described herein can be assessed for their ability to inhibit tumor growth, progression, and metastasis in standard in vivo tumor growth and metastasis models. See, e.g., Rockwell et al., J Natl. Cancer Inst., 49:735 (1972); Guy et al., Mol. Cell Biol., 12:954 (1992); Wyckoff et al., Cancer Res., 60:2504 (2000); and Oft et al., Curr. Biol., 8:1243 (1998).

The efficacy of treatments may be measured by a number of available diagnostic tools, including physical examination, blood tests, proteinuria measurements, creatinine levels and creatinine clearance, pulmonary function tests, plasma blood urea nitrogen (BUN) levels, observation and scoring of scarring or fibrotic lesions, deposition of extracellular matrix such as collagen, smooth muscle actin and fibronectin, kidney function tests, ultrasound, magnetic resonance imaging (MRI), and CT scan.

### Pharmaceutical Compositions

The anti-integrin antibodies described herein can be formulated as a pharmaceutical composition for administration to a subject, e.g., to treat a disease or condition described herein. Typically, a pharmaceutical composition includes a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. The composition can include a pharmaceutically acceptable salt, e.g., an acid addition salt or a base addition salt (see e.g., Berge, S. M., et al. (1977) J. Pharm. Sci. 66:1-19).

Pharmaceutical formulation is a well-established art, and is further described, e.g., in Gennaro (ed.), Remington: The Science and Practice of Pharmacy, 20^{th} ed., Lippincott, Williams & Wilkins (2000) (ISBN: 0683306472); Ansel et al., Pharmaceutical Dosage Forms and Drug Delivery Systems, 7^{th} Ed., Lippincott Williams & Wilkins Publishers (1999) (ISBN: 0683305727); and Kibbe (ed.), Handbook of Pharmaceutical Excipients American Pharmaceutical Association, 3^{rd} ed. (2000) (ISBN: 091733096X).

The pharmaceutical compositions may be in a variety of forms. These include, for example, liquid, semi-solid and solid dosage forms, such as liquid solutions (e.g., injectable and infusible solutions), dispersions or suspensions, tablets, pills, powders, liposomes and suppositories. The preferred form can depend on the intended mode of administration and therapeutic application. Typically compositions for the agents described herein are in the form of injectable or infusible solutions.

Such compositions can be administered by a parenteral mode (e.g., intravenous, subcutaneous, intraperitoneal, or intramuscular injection). In one embodiment, the antibody composition is administered intravenously. In another embodiment, the antibody composition is administered subcutaneously. The phrases “parenteral administration” and “administered parenterally” as used herein mean modes of administration other than enteral and topical administration, usually by injection, and include, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

The composition can be formulated as a solution, microemulsion, dispersion, liposome, or other ordered structure suitable for stable storage at high concentration. Sterile injectable solutions can be prepared by incorporating an agent described herein in the required amount in an appropriate solvent with one or a combination of ingredients enumerated above, as required, followed by filtered sterilization. Generally, dispersions are prepared by incorporating an agent described herein into a sterile vehicle that contains a basic dispersion medium and the required other ingredients from those enumerated above. In the case of sterile powders for the preparation of sterile injectable solutions, the preferred methods of preparation are vacuum drying and freeze drying that yield a powder of an agent described herein plus any additional desired ingredient from a previously sterile-filtered solution thereof.

### Administration

The antibody described herein can be administered to a subject, e.g., a human subject in need thereof, for example, by a variety of methods. For many applications, the route of administration is one of: intravenous injection or infusion (IV), subcutaneous injection (SC), intraperitoneally (IP), or intramuscular injection. It is also possible to use intra-articular delivery. Other modes of parenteral administration can also be used. Examples of such modes include: intraarterial, intrathecal, intracapsular, intraorbital, intracardiac, intradermal, transtracheal, subcuticular, intraarticular, subcapsular, subarachnoid, intraspinal, and epidural and intrasternal injection. In some cases, administration can be oral.

The route and/or mode of administration of the antibody or antigen-binding fragment thereof can also be tailored for the individual case, e.g., by monitoring the subject, e.g., using tomographic imaging, e.g., to visualize a tumor.

If a subject is at risk for developing a disease or condition described herein, the antibody can be administered before the full onset of the disease or condition, e.g., as a preventative measure. The duration of such preventative treatment can be a single dosage of the antibody or the treatment may continue (e.g., multiple dosages). For example, a subject at risk for the disease or who has a predisposition for the disease may be treated with the antibody for days, weeks, months, or even years so as to prevent the disease from occurring or fulminating.

A pharmaceutical composition may include a “therapeutically effective amount” of an agent described herein. Such effective amounts can be determined based on the effect of the administered agent, or the combinatorial effect of agents if more than one agent is used. A therapeutically effective amount of an agent may also vary according to factors such as the disease state, age, sex, and weight of the individual, and the ability of the compound to elicit a desired response in the individual, e.g., amelioration of at least one disease or condition parameter or amelioration of at least one symptom of the disease or condition. A therapeutically effective amount is also one in which any toxic or detrimental effects of the composition are outweighed by the therapeutically beneficial effects.

### Devices and Kits for Therapy

Pharmaceutical compositions that include the antibody described herein can be administered with a medical device. The device can be designed with features such as portability, room temperature storage, and ease of use so that it can be used in emergency situations, e.g., by an untrained subject or by emergency personnel in the field, removed from medical facilities and other medical equipment. The device can include, e.g., one or more housings for storing pharmaceutical preparations that include the antibody, and can be configured to deliver one or more unit doses of the antibody. The device can be further configured to administer a second agent either as a single pharmaceutical composition that also includes the antibody described herein or as two separate pharmaceutical compositions.

The pharmaceutical composition may be administered with a syringe. The pharmaceutical composition can also be administered with a needleless hypodermic injection device, such as the devices disclosed in U.S. Pat. Nos. 5,399,163; 5,383,851; 5,312,335; 5,064,413; 4,941,880; 4,790,824; or 4,596,556. Examples of well-known implants and modules include: U.S. Pat. No. 4,487,603, which discloses an implantable micro-infusion pump for dispensing medication at a controlled rate; U.S. Pat. No. 4,486,194, which discloses a therapeutic device for administering medicaments through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. Many other devices, implants, delivery systems, and modules are also known.

An antibody described herein can be provided in a kit. In one embodiment, the kit includes (a) a container that contains a composition that includes an antibody described herein, and optionally (b) informational material. The informational material can be descriptive, instructional, marketing or other material that relates to the methods described herein and/or the use of the agents for therapeutic benefit.

In an embodiment, the kit also includes a second agent for treating a disease or condition described herein. For example, the kit includes a first container that contains a composition that includes the antibody described herein, and a second container that includes the second agent.

The informational material of the kits is not limited in its form. In one embodiment, the informational material can include information about production of the compound, molecular weight of the compound, concentration, date of expiration, batch or production site information, and so forth. In one embodiment, the informational material relates to methods of administering the antibody described herein, e.g., in a suitable mode of administration (e.g., a mode of administration described herein), to treat a subject who has had or who is at risk for a disease or condition described herein. The information can be provided in a variety of formats, include printed text, computer readable material, video recording, or audio recording, or information that provides a link or address to substantive material, e.g., on the internet.

In addition to the antibody, the composition in the kit can include other ingredients, such as a solvent or buffer, a stabilizer, or a preservative. The antibody can be provided in any form, e.g., liquid, dried or lyophilized form, preferably substantially pure and/or sterile. When the agents are provided in a liquid solution, the liquid solution preferably is an aqueous solution. When the agents are provided as a dried form, reconstitution generally is by the addition of a suitable solvent. The solvent, e.g., sterile water or buffer, can optionally be provided in the kit.

The kit can include one or more containers for the composition or compositions containing the agents. In some embodiments, the kit contains separate containers, dividers or compartments for the composition and informational material. For example, the composition can be contained in a bottle, vial, or syringe, and the informational material can be contained in a plastic sleeve or packet. In other embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of the agents. The containers can include a combination unit dosage, e.g., a unit that includes both the antibody described herein and the second agent, e.g., in a desired ratio. For example, the kit includes a plurality of syringes, ampules, foil packets, blister packs, or medical devices, e.g., each containing a single combination unit dose. The containers of the kits can be air tight, waterproof (e.g., impermeable to changes in moisture or evaporation), and/or light-tight. The kit optionally includes a device suitable for administration of the composition, e.g., a syringe or other suitable delivery device. The device can be provided pre-loaded with one or both of the agents or can be empty, but suitable for loading.

### Methods of Selecting an Anti-Integrin Antibody of Interest

Some aspects of the disclosure provide methods of selecting, discovering or isolating an antibody of interest using any of the anti-integrin antibodies described herein. The antibody of interest can be antibodies that bind to αvβ1 integrin but no other integrin (e.g., other αv- or β1-containing or RGD family integrins), those that bind to αvβ1 and αvβ6 integrins but no other integrins, or those that bind to αvβ1 and one or more integrins selected from the group consisting of αvβ3, αvβ5, αvβ6, αvβ8, α5β1, α8β1, and αIIBβ3.

By way of example, to select anti-integrin antibodies of interest, a guiding selection process can be carried out using guiding antibodies, which can be antibodies described herein that bind to αvβ1 integrin but no other integrin (e.g., other αv- or β1-containing or RGD family integrins), such as Exemplary antibodies 1-10. For example, a labeled recombinant or purified αvβ1 integrin (e.g., a polypeptide or polypeptides comprising the extracellular domains of αv and β1) and a prokaryotic or eukaryotic (e.g., yeast) antibody expression library can be provided. Clones in the antibody expression library that show reduced binding to the labeled antigen upon addition of the guiding antibody can be selected, which are enriched for the antibodies of interest. Additional descriptions of the guiding selection process can be found at e.g., Cherf and Cochran, Methods Mol Biol. 1319:155-75, 2015; Xu et al. Protein Eng Des Sel. 26(10):663-70, 2013; and Mann et al. ACS Chem Biol. 8(3):608-16, 2013. In some instances, any one or more of Exemplary Antibodies 11-14 and 15-20 can also be used as guiding antibodies to select, discover, or isolate an antibody of interest (e.g., antibodies that bind to αvβ1 integrin but no other integrin (e.g., other αv- or β1-containing or RGD family integrins), those that bind to αvβ1 and αvβ6 integrins but no other integrins, or those that bind to αvβ1 and one or more integrins selected from the group consisting of αvβ3, αvβ5, αvβ6, αvβ8, α5β1, α8β1, and αIIBβ3).

To select antibodies that bind to αvβ1 integrin but no other integrin (e.g., other αv- or β1-containing or RGD family integrins), for example, the methods can further include depletion of antibodies that bind to undesired integrins, e.g., αv- or β1-containing or RGD family integrins including αvβ3, αvβ5, αvβ6, αvβ8, α5β1, α8β1, and αIIBβ3. The methods can also include positive selection steps using the target integrin of interest, the αvβ1 integrin. Antibodies that bind to αvβ1 and αvβ6 integrins but no other integrins can be similarly selected by depleting antibodies that bind to integrins other than αvβ1 and αvβ6, and/or by performing positive selections using αvβ1 and αvβ6 integrins. Such principles also apply to the selection of antibodies that bind to αvβ1 and one or more integrins selected from the group consisting of αvβ3, αvβ5, αvβ6, αvβ8, α5β1, α8β1, and αIIBβ3.

Antibodies enriched through one or more of the above steps can also be subjected to affinity maturation to increase affinity and specificity for the target integrin, building libraries using methods known in the art for affinity optimization (e.g., light chain shuffling or H-CDR1/H-CDR-2 targeted mutagenesis).

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art can develop equivalent means or reactants without the exercise of inventive capacity and without departing from the scope of the invention.

### Example 1: Design of Antibody Selections and Antibody Production

The integrin αvβ1 is known to bind to several extracellular matrix proteins. The heterodimeric complex is a combination of the alpha subunit αv and beta subunit β1 (SEQ ID NOs: 1 and 2). The αv subunit is capable of functional pairing with four additional beta subunits: β3, β5, β6, and β8. The β1 subunit is capable of functional pairing with eleven additional alpha subunits: α1, α2, α3, α4, α5, α6, α7, α8, α9, α10, α11 (Hynes R O, Cell, 110(6):673-87 (2002)).

The integrin αvβ1 was recombinantly expressed and purified according to methods known in the art. Additionally, the integrins αvβ3, αvβ5, αvβ6, αvβ8, α4β1, α5β1, and α8β1 were recombinantly expressed and purified according to methods known in the art. This disclosure describes three different groups of antibodies: antibodies that bind to αvβ1 integrin but no other integrin (e.g., no other αv or β1-containing integrins); antibodies that bind to αvβ1 and αvβ6 integrins but no other integrins; and antibodies that bind to αvβ1 and one or more integrins selected from the group consisting of αvβ3, αvβ5, αvβ6, αvβ8, α5β1, α8β1, and αIIBβ3.

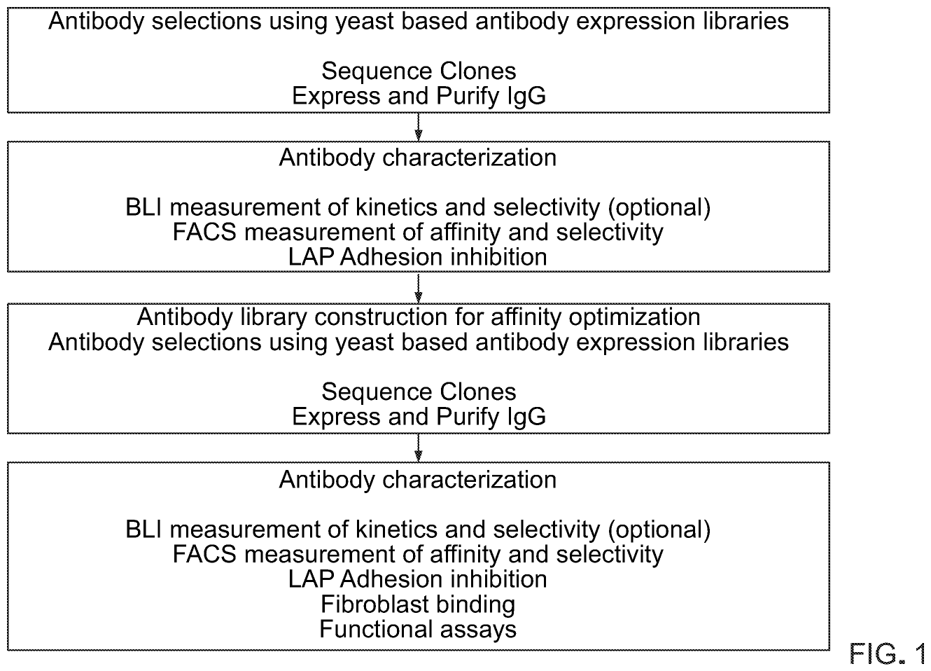

To generate these groups of antibodies, Adimab expression libraries were screened in accordance with the methods disclosed in US Patent Publications 20100056386 and 20090181855. Multiple iterative rounds of selective pressure towards the antigen of interest αvβ1 (SEQ ID NOs: 1 and 2) and selective pressure to diminish binding to undesired antigens αvβ3, αvβ5, αvβ6, αvβ8, α4β1, α5β1, and α8β1 were performed. In the selections where binding to αvβ1 and αvβ6 is desirable, iterative rounds of selective pressure towards αvβ6 were introduced and rounds to diminish binding to αvβ6 were eliminated. Selections can also be designed to guide to an epitope of interest using guiding proteins (i.e. mAbs, Fabs, or ligands). Selections were performed in the presence and absences of cations, including calcium, magnesium, and manganese. After selections were completed, colonies were sequenced to identify unique clones using technique known in the art. Following four campaigns, over 2500 antibodies were expressed and purified on protein A resin from yeast using methods known in the art. A general outline for the triage of αvβ1-specific, αvβ1l/αvβ6-specific, and αvβ1 plus one or more integrin-binding antibodies is depicted in FIG. 1.

Antibody optimization was also performed to increase the affinity of certain antibodies for αvβ1 as well as fine-tune integrin specificity. Antibody libraries were built using methods known in the art for affinity optimization (i.e., Light Chain shuffling and H-CDR1/H-CDR-2 targeted mutagenesis). Multiple iterative rounds of selection pressure towards the antigen of interest was applied using decreasing concentrations of αvβ1 recombinant protein. Selective pressure to diminish binding to undesired antigens αvβ3, αvβ5, αvβ6, αvβ8, α4β1, α5β1, and α8β1 was performed. After selections were completed, colonies were sequenced to identify unique clones using techniques known in the art. Following antibody optimization campaigns, over 500 antibodies were expressed and purified on protein A resin from yeast using methods known in the art.

This analysis led to the identification of twenty antibodies. The amino acid sequences of the CDRs and variable regions of these antibodies are provided herein.

### Example 2: Determination of Binding Kinetics and Integrin Specificity

After campaigns 1-3, antibodies were initially screened for positive binding to αvβ1 in the presence or absence of cations. Positive antibodies were subsequently screened for binding to other αv-containing and β1-containing integrins. This screening step was performed to focus future characterization on antibodies that recognize the combination of the αv and β1 subunits and to eliminate antibodies that recognized only the αv subunit or the β1 subunit. This step also allows stratification of antibodies based on a preference for binding to RGD binding integrins (i.e., αvβ1, αvβ3, αvβ5, αvβ6, αvβ8, α5β1, α8β1, αIIBβ3) and non-RGD binding 31 containing integrins (i.e., α4β1).

Antibodies were screened for binding to target antigen using Bio-Layer Interferometry (BLI). BLI was performed on the Octet RED384 and Octet HTX instruments manufactured by ForteBio according to standard procedures. A subset of antibodies was classified based on specificity for subsequent screening in additional assays.

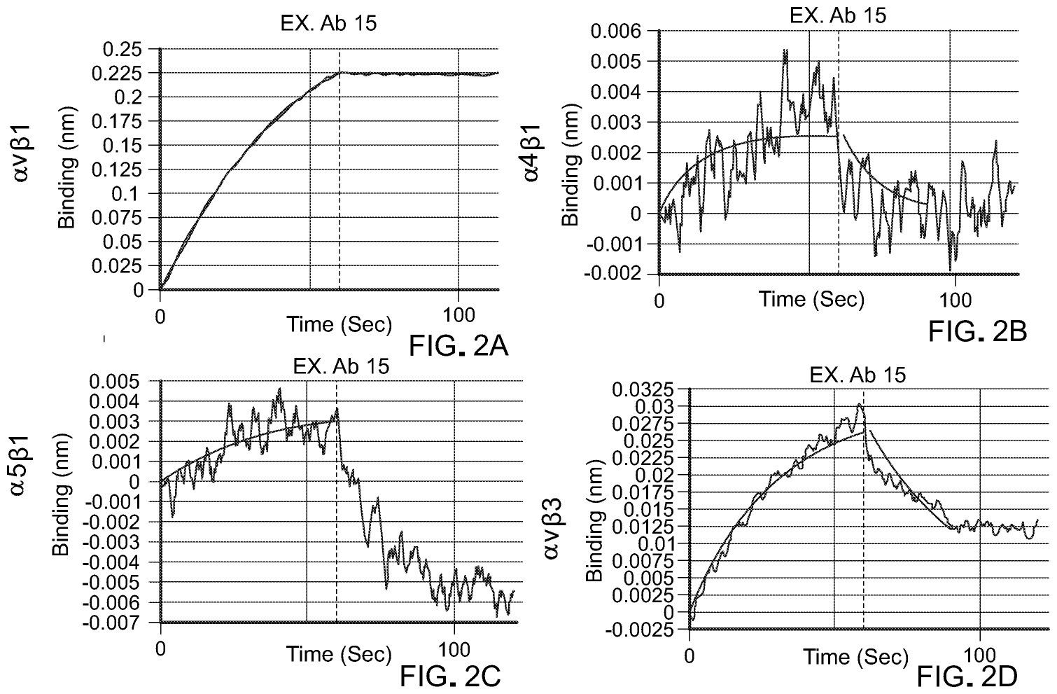

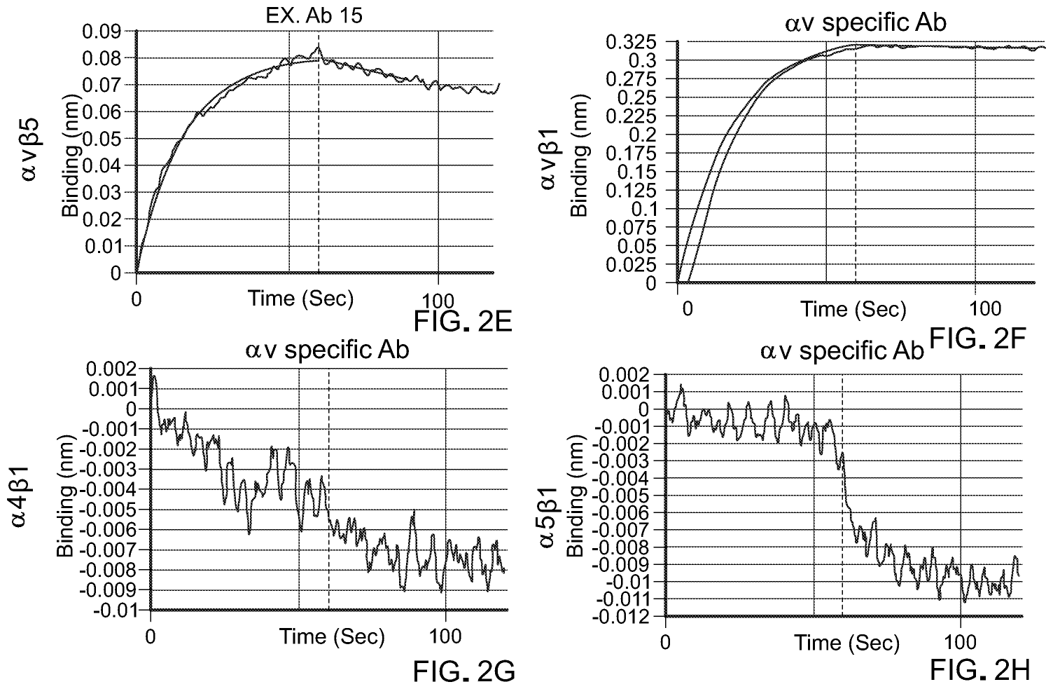

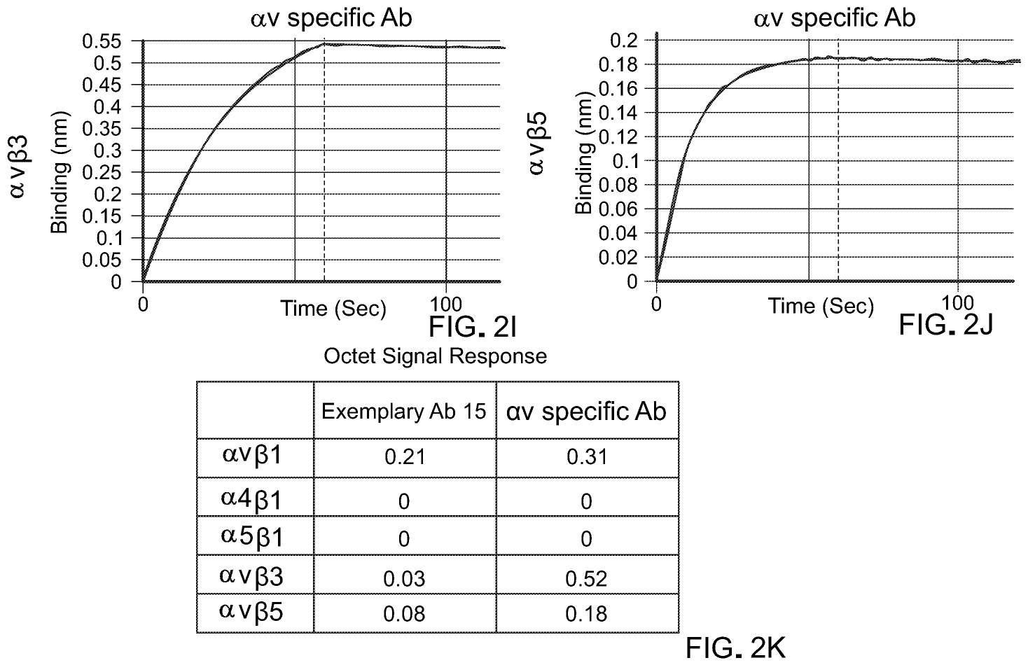

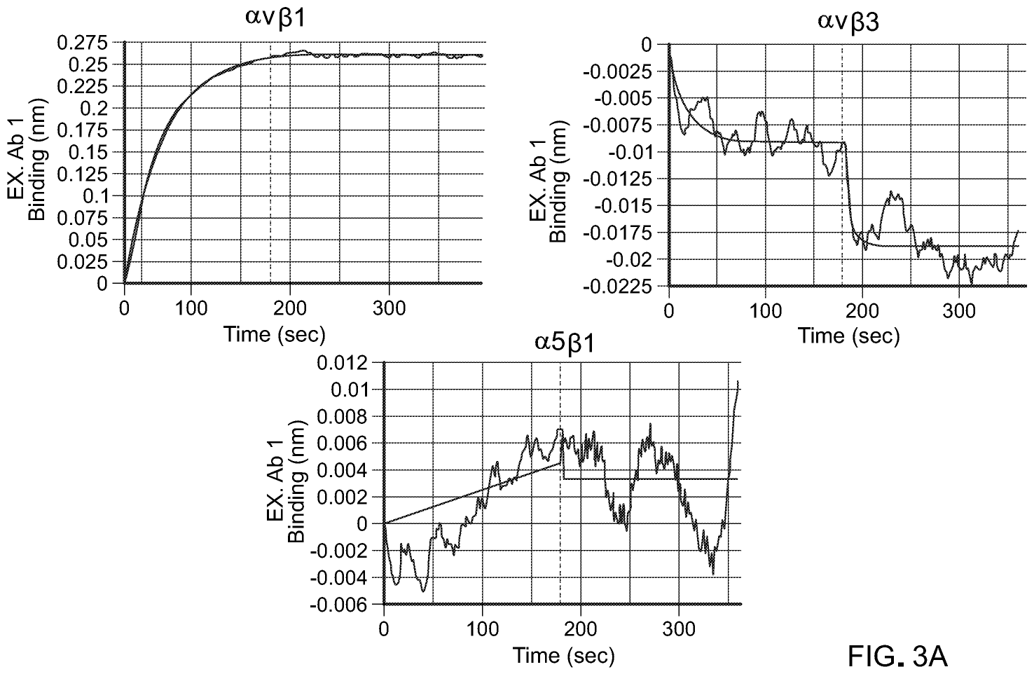

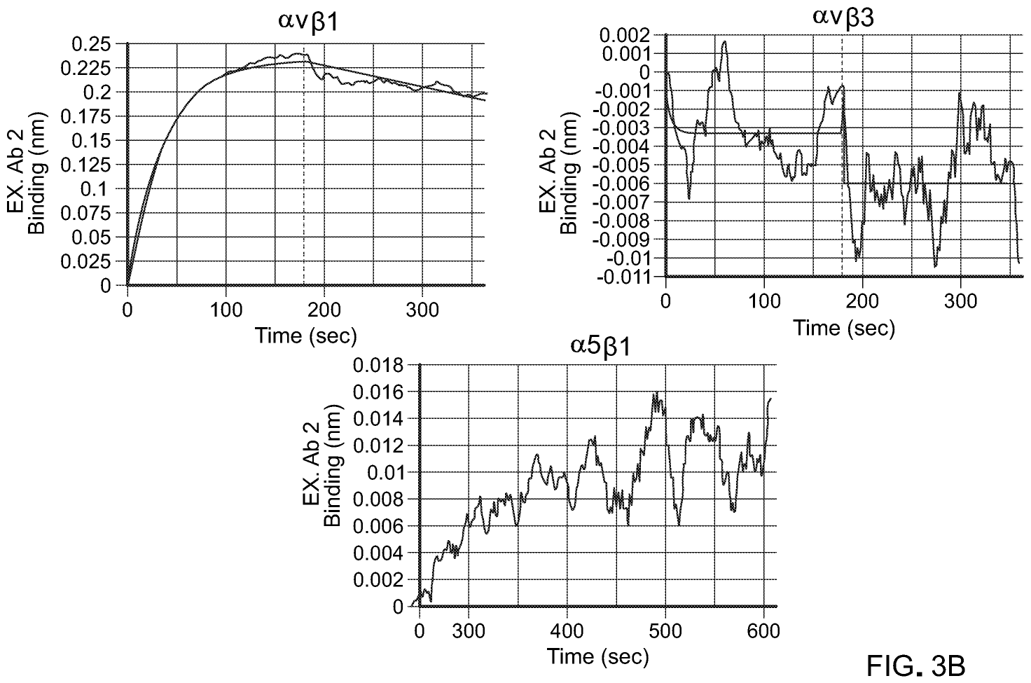

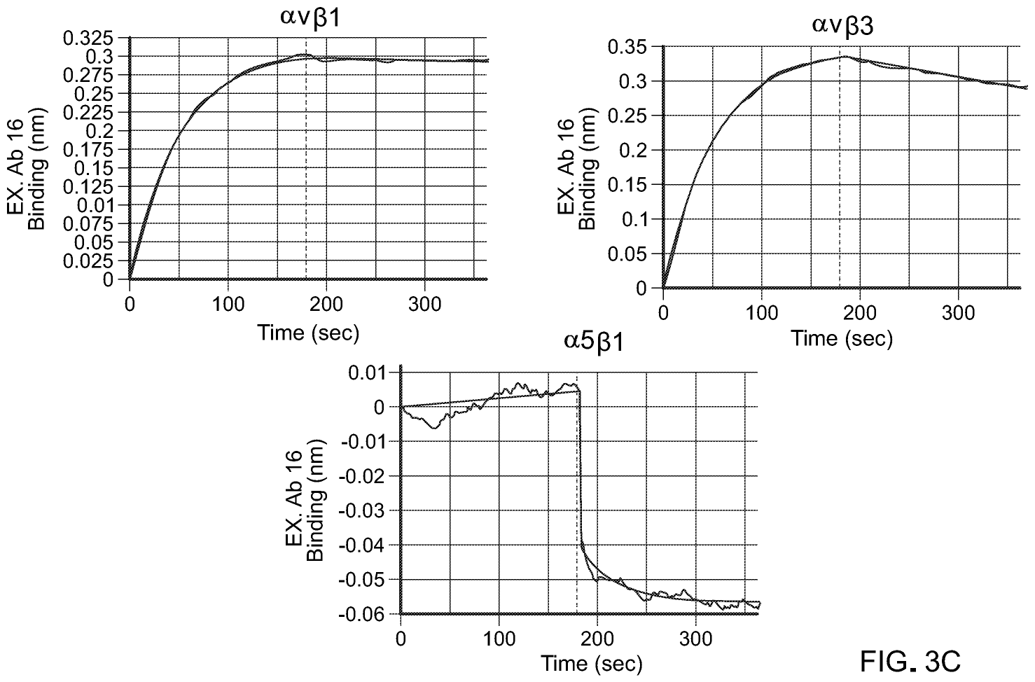

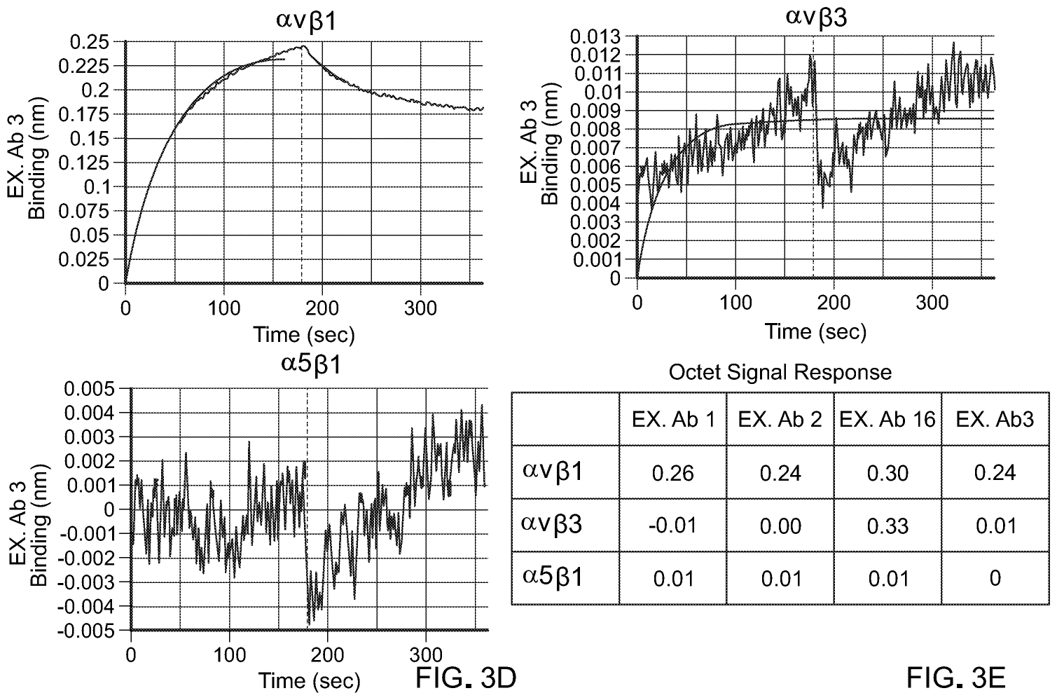

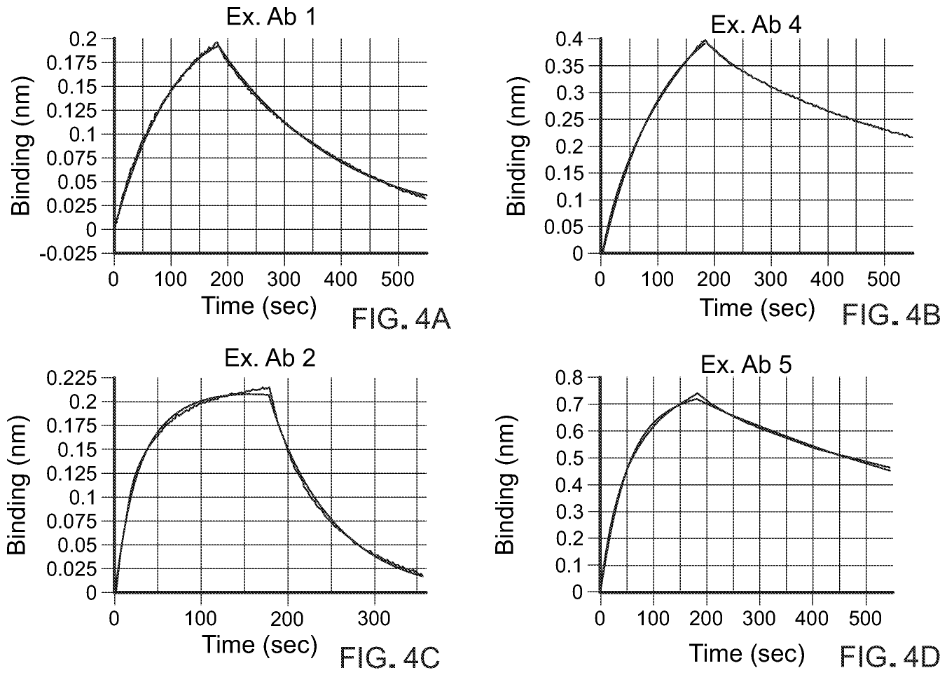

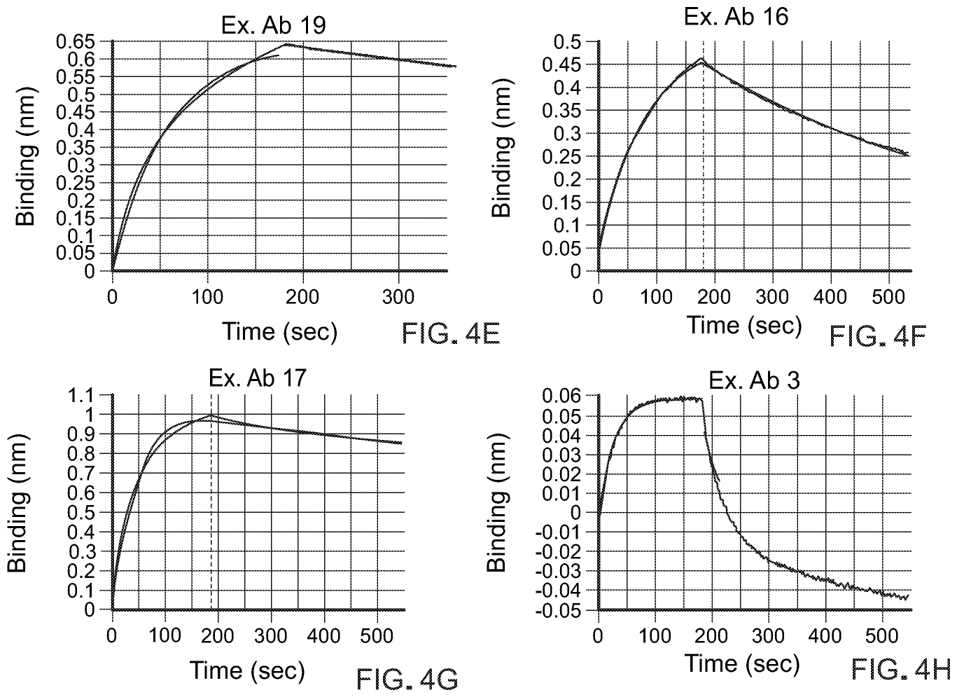

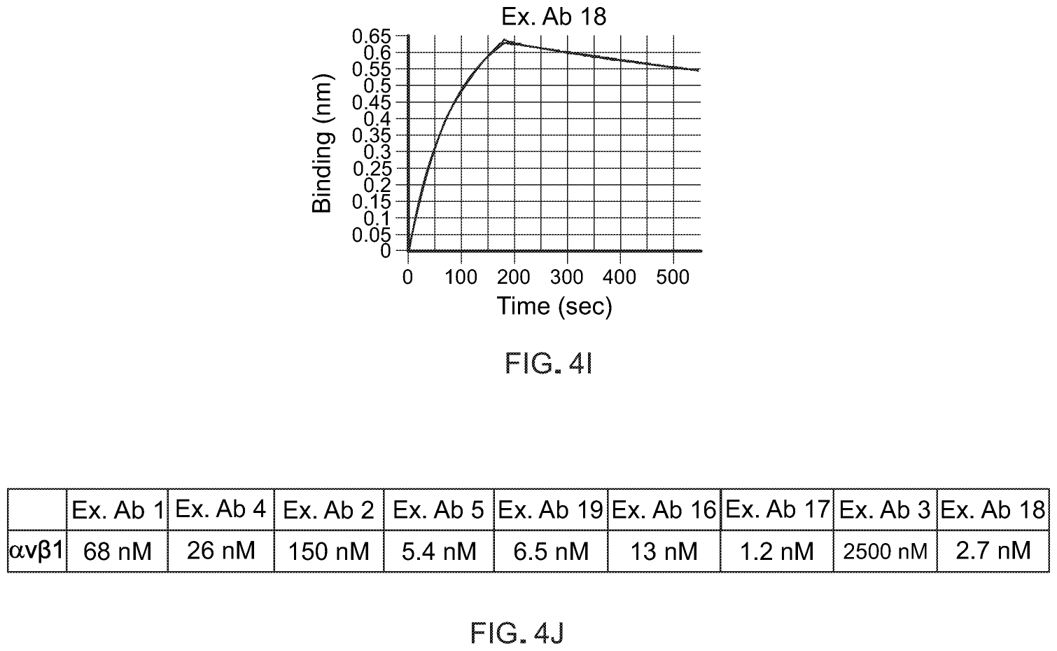

Examples of observed binding kinetics for αvβ1-specific, non-specific, and partially selective antibodies are shown in FIGS. 2A-K and FIGS. 3A-E. Additionally, monovalent binding affinity for recombinant αvβ1 is shown in FIGS. 4A-J. This includes antibodies from the original three campaigns and subsequent affinity optimization campaigns. Monovalent affinity screening after affinity maturation allowed selection of the highest affinity clones from optimization for further characterization.

Examples of antibodies that exhibit specificity for αvβ1 include Exemplary Antibody 1 and Exemplary Antibody 2. Examples of antibodies that are partially selective include Exemplary Antibody 15 and Exemplary Antibody 16. Higher affinity antibodies selected after affinity maturation include Exemplary Antibody 4 and Exemplary Antibody 5.

### Example 3: Determination of Cell-Surface Binding and Integrin Specificity

Stably transfected cells expressing αvβ1, αvβ3, αvβ5, αvβ6, αvβ8, α4β1, α5β1, and α8β1 were made by methods known in the art.

After campaigns 1-3, antibodies were initially screened by Octet RED384 and Octet HTX. A subset of antibodies was selected for additional specificity screening and affinity measurement against the transfected cells and untransfected cells. After campaign 4, antibodies were screened directly on stably transfected cells expressing αvβ1. Positive antibodies were subsequently screened for binding to other αv-containing and β1-containing stably transfected cells. This screening step was performed to focus future characterization on antibodies that recognize the combination of the αv and β1 subunits and to eliminate antibodies that recognized only the αv subunit or the β1 subunit. This step also allows stratification of antibodies based on a preference for binding to RGD binding integrins (i.e. αvβ1, αvβ3, αvβ5, αvβ6, αvβ8, α5β1, α8β1, αIIBβ3) and non-RGD binding β1 containing integrins (i.e. α4β1).

After initial cell binding screening at one to three concentrations, 6 or 11-pt titrations were performed on multiple αv-containing and β1-containing cell-lines.

Cells were harvested and viability was confirmed to be greater than 90%. Cells were washed in the appropriate assay buffer three times by pelleting at 1500 RPM for 3 minutes and then pouring out supernatant. Assay buffer for this experiment was TBS containing 1 mg/mL BSA and supplemented with either 1 mM Ca+1 mM Mg or with 1 mM Mn. Cells were then resuspended in assay buffer at a concentration of 1.0-1.5×10⁶ cells/mL and transferred to wells of a 96-well assay plate (Corning 3799) in 50 μL aliquots. Plates were then centrifuged at 2000 RPM for 3 minutes to pellet cells and supernatant was flicked out. Cells were resuspended in 100 μL of antibody sample and incubated on ice for 45 to 60 minutes. Lower antibody concentrations called for longer incubation times.

Plates were washed three times in 150 μL assay buffer by pelleting at 2000 RPM for 3 minutes and then flicking out the supernatant. Cells were then resuspended in 50 μL of phycoerythrin-conjugated goat anti-human Fab secondary reagent and incubated on ice in the dark for 30 minutes. Plates were washed three times in 150 μL assay buffer by pelleting at 2000 RPM for 3 minutes and then flicking out the supernatant. Cells were then resuspended in 200 μL of assay buffer+1% polyformaldehyde (PFA) for 30 minutes on ice to fix the cells. Cells were pelleted to remove the PFA and then resuspended in 150 μL assay buffer.

Cell populations were analyzed on a BD FACSCALIBUR flow cytometer.

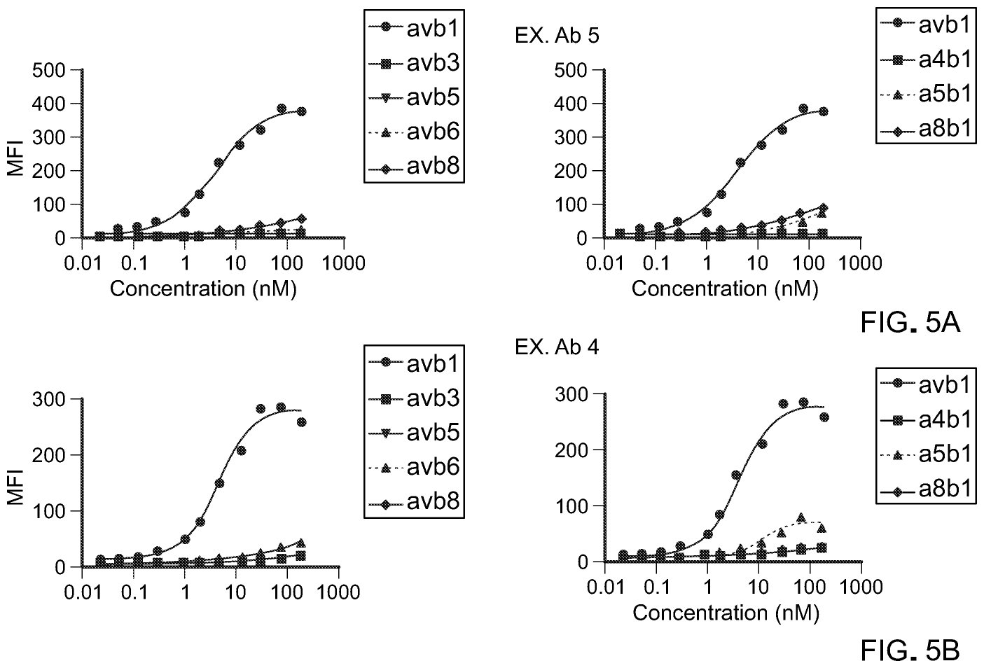

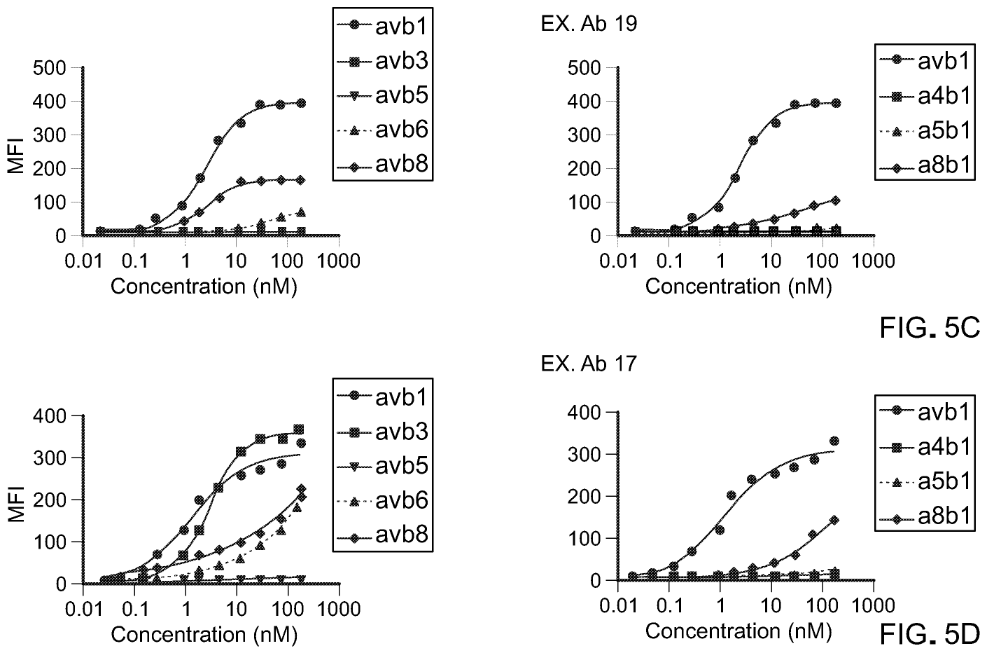

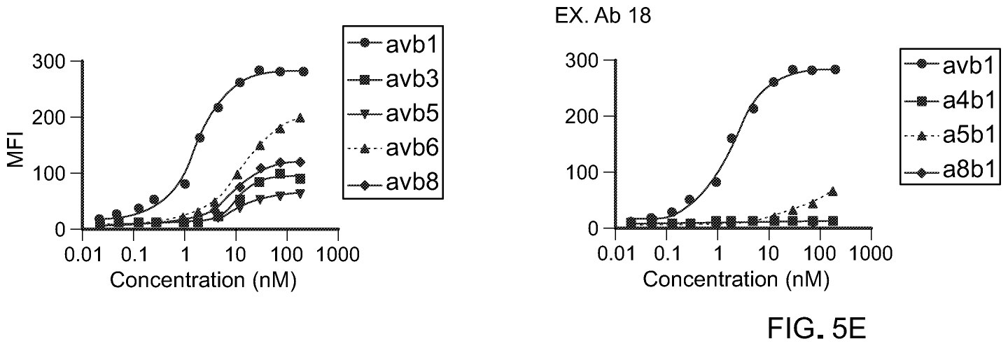

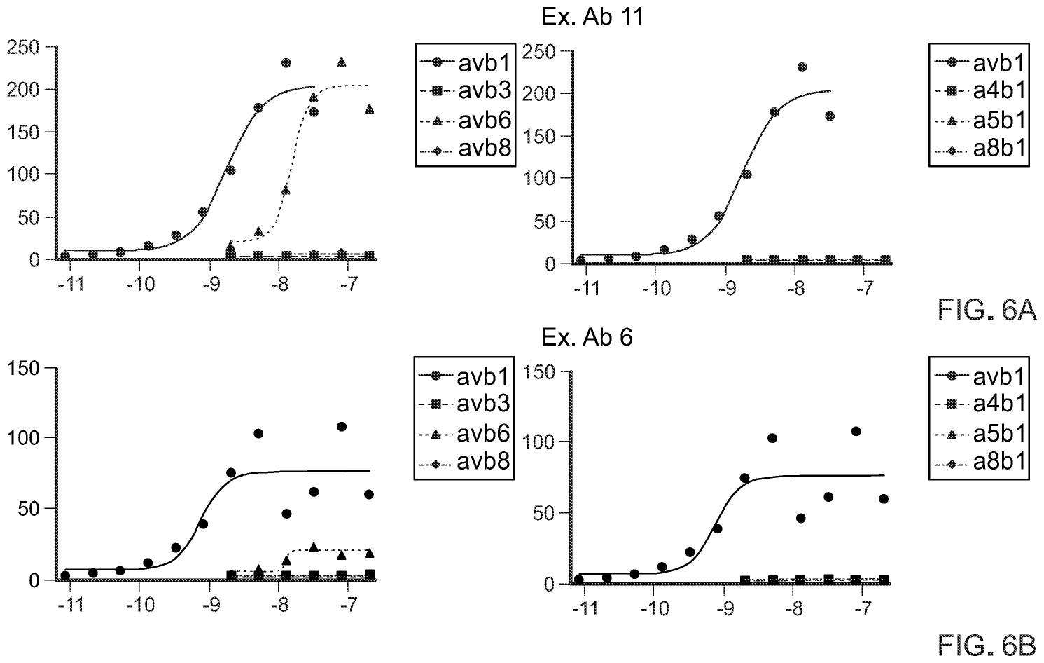

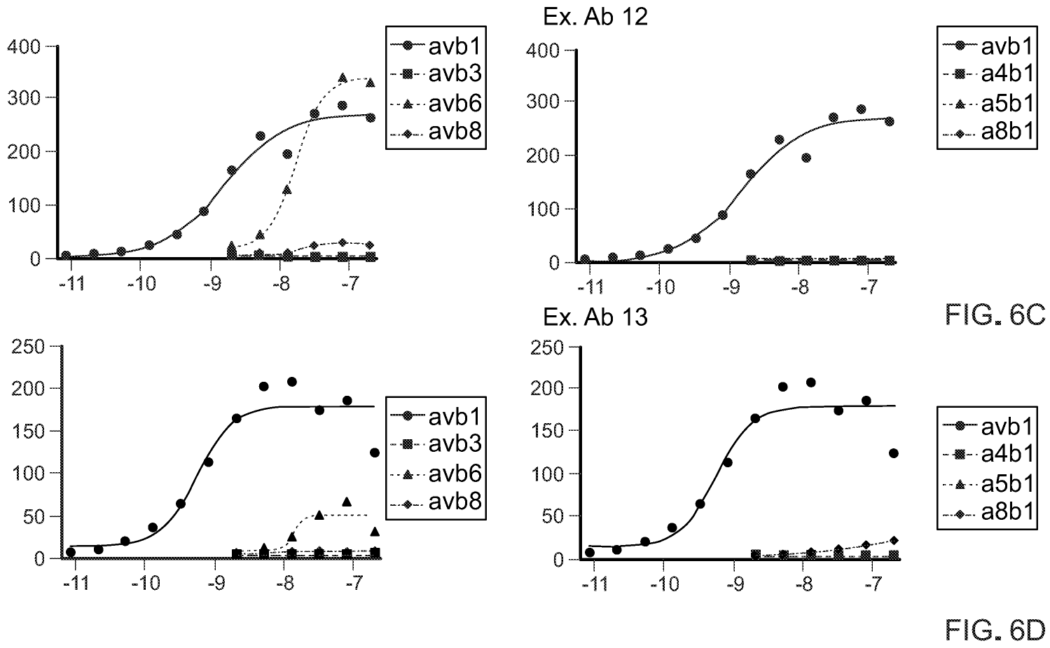

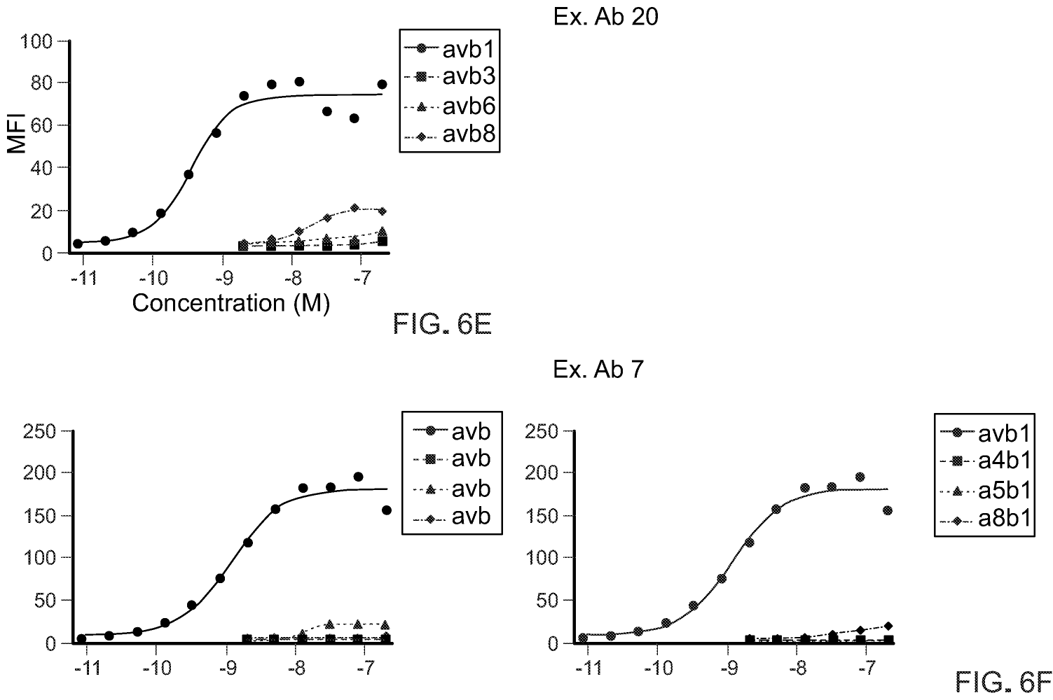

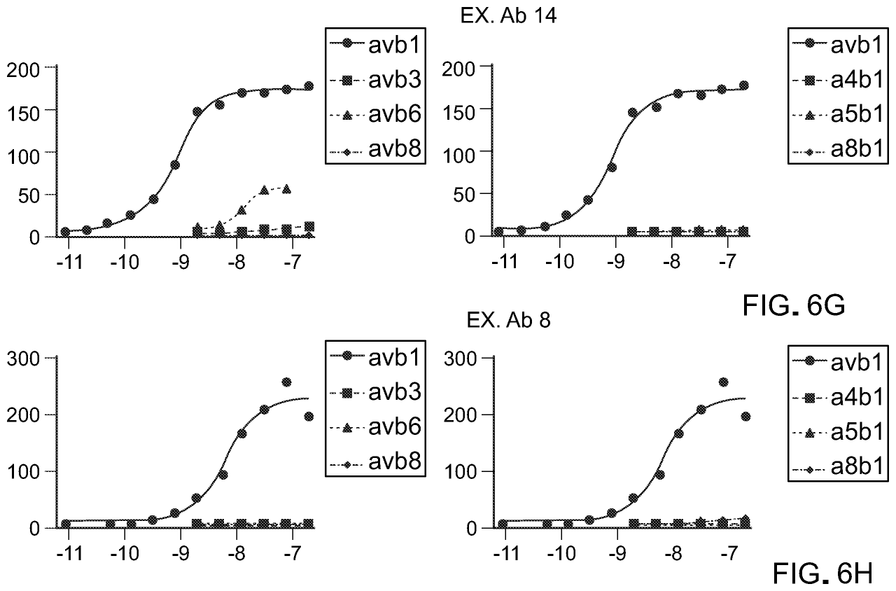

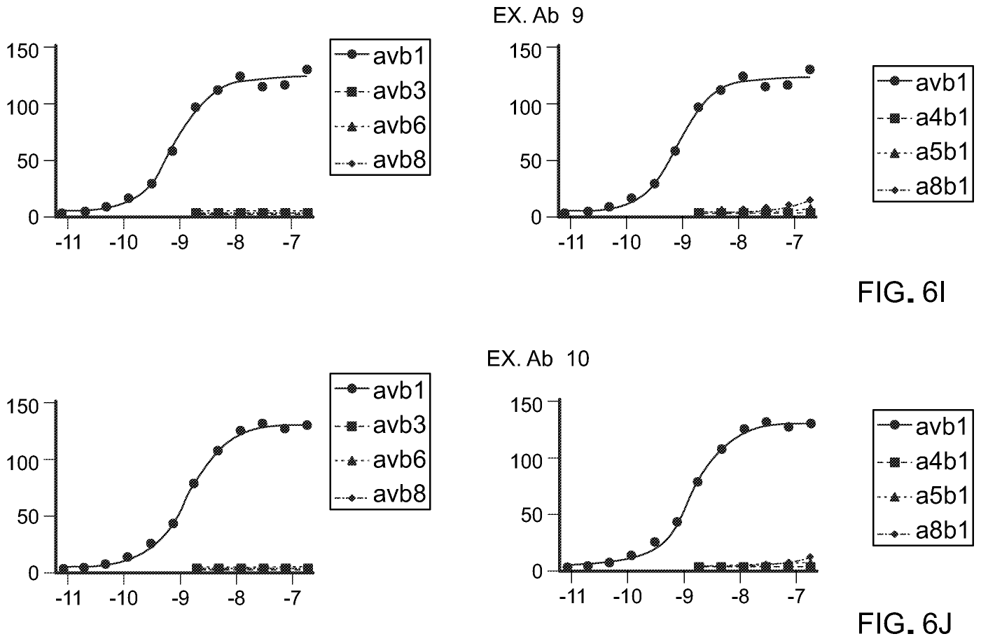

Examples of observed binding titrations for αvβ1-specific and partially selective antibodies are shown in FIGS. 5A-E and FIGS. 6A-J. This includes antibodies from the original four campaigns and subsequent affinity optimization campaigns. The bivalent affinity (i.e., EC50) for cell-surface binding is summarized for a subset of RGD binding integrins in TABLE 1.

TABLE 1

| | EC50 | EC50 | EC50 | EC50 | EC50 | EC50 |
|---|---|---|---|---|---|---|
| | αvβ1 | αvβ3 | αvβ5 | αvβ6 | αvβ8 | α8β1 |
| Ab | (nM) | (nM) | (nM) | (nM) | (nM) | (nM) |
| Ex. Ab 5 | 4.5 | — | — | — | — | — |
| Ex. Ab 4 | 4.9 | — | — | — | — | — |
| Ex. Ab 19 | 2.8 | — | — | — | 2.8 | — |
| Ex. Ab 17 | 1.4 | 3.3 | — | >100 | >100 | >100 |
| Ex. Ab 18 | 2.0 | 13 | 11 | 16 | 10 | — |
| Ex. Ab 11 | 1.9 | — | n.d. | 16 | — | — |
| Ex. Ab 6 | 0.8 | — | n.d. | binding | — | — |
| Ex. Ab 12 | 1.6 | — | n.d. | 17 | — | — |
| Ex. Ab 13 | 0.6 | — | n.d. | binding | — | — |
| Ex. Ab 20 | 0.4 | — | n.d. | — | binding | n.d. |
| Ex. Ab 7 | 1.2 | — | n.d. | binding | — | — |
| Ex. Ab 14 | 0.8 | — | n.d. | binding | — | — |
| Ex. Ab 8 | 6.3 | — | n.d. | — | — | — |
| Ex. Ab 9 | 0.9 | — | n.d. | — | — | — |
| Ex. Ab 10 | 1.5 | — | n.d. | — | — | — |

Key:
n.d. = not determined;
binding = weak binding observed but incomplete fit to calculate EC50

Examples of antibodies that exhibit specificity for αvβ1 include Exemplary Antibody 5, Exemplary Antibody 4, Exemplary Antibody 7, and Exemplary Antibody 8. Examples of antibodies that exhibit specificity for both αvβ1 and αvβ6 include Exemplary Antibody 11, Exemplary Antibody 12, and Exemplary Antibody 14. Examples of antibodies that are partially selective (i.e., bind to αvβ1 and one or more integrins selected from the group consisting of αvβ3, αvβ5, αvβ6, αvβ8, α5β1, α8β1, and αIIBβ3) include Exemplary Antibody 19 and Exemplary Antibody 17.

### Example 4: αvβ1 Latency-Associated Peptide Adhesion Inhibition

After specific and partially selective antibodies were determined, the antibodies were tested in a Latency-Associated Peptide (LAP) adhesion assay to confirm the ability to block integrin/ligand interaction to focus on antibodies that can disrupt functional biological activity. LAP is one of many ligands for the αvβ1 heterodimer, as well as other integrin heterodimers, including αvβ6 and αvβ8.

A 96-well microtiter plate (Costar 3369) was coated with 100 μl/well of 10 μg/ml LAP (R&D Systems, Cat. #246-LP) diluted in 50 mM sodium bicarbonate, pH 9.2, at 4° C. overnight. The plate was washed twice with PBS (200 μl/well), blocked with 1% BSA in PBS (200 μl/well) for 1 h at room temperature, and washed thrice with 200 μl/well of assay buffer (TBS, 2 mM Glucose, 0.1% BSA, 1 mM CaCl₂) and 1 mM MgCl₂, pH 7.4). Stably transfected human αvβ1 cells were removed from cell suspension and centrifuged at 1100 rpm for 5 mins. The pellet was resuspended in RPMI 1640, 1% BSA buffer and incubated with 2 μM fluorescent dye (BCECF, Molecular Probes, Eugene, Oreg.) in a 37° C. incubator for 30 min. The labeled cells were washed twice by centrifugation at 1100 rpm for 5 min and resuspended in assay buffer to 0.8×10⁶ cells/ml. To individual wells of the washed plate were added 50 μl of purified antibody and 50 μl of human αvβ1 cells labeled with BCECF, and the plate was incubated at room temperature in dark for 1 h. The plate was washed 3-4 times with assay buffer (200 μl/well), and the fluorescence due to adhered cells on the plate was recorded at 485 nm (Excitation) and 538 nm (Emission) wavelength. Percent binding was determined by comparing the fluorescence prior to the final wash step (i.e. total cells added) to that after washing (i.e. bound cells).

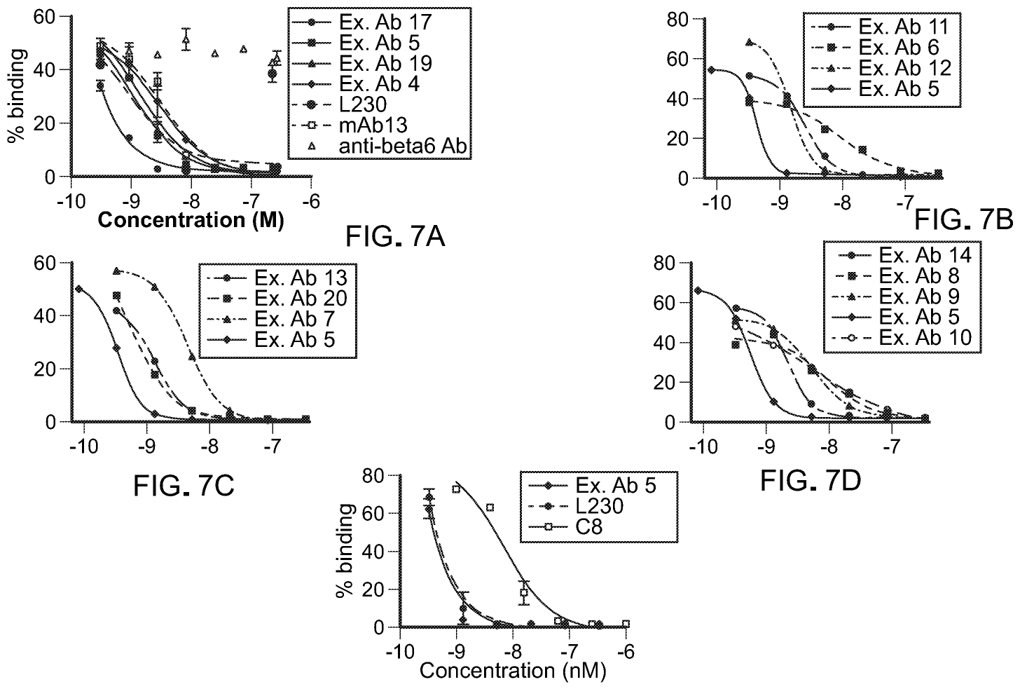

Examples of αvβ1 LAP adhesion inhibition are shown in FIGS. 7A-E. This includes examples for a pan-αv commercial antibody (L230), a pan-β1 commercial antibody (mAb13), a published β6-specific antibody, and the c8 small molecule compound previously published as αvβ1 specific (Reed N I et al, Sci Transl Med, 7(288): 288ra79 (2015)). The inhibition of cell-based adhesion (i.e., IC50) is summarized for all antibodies in TABLE 2.

TABLE 2

| Antibody | IC50 αvβ1 (nM) |
|---|---|
| Ex. Ab 5 | 1.0 |
| Ex. Ab 4 | 2.8 |
| Ex. Ab 19 | 1.4 |
| Ex. Ab 17 | <1.0 |
| Ex. Ab 18 | No blocking |
| Ex. Ab 5 | 0.4, 0.3, 0.5 |
| (triplicate) | |
| Ex. Ab 11 | 2.5 |
| Ex. Ab 6 | 9.0 |
| Ex. Ab 12 | 1.5 |
| Ex. Ab 13 | 1.3 |
| Ex. Ab 20 | 1.0 |
| Ex. Ab 7 | 4.3 |
| Ex. Ab 14 | 2.2 |
| Ex. Ab 8 | 9.0 |
| Ex. Ab 9 | 5.4 |
| Ex. Ab 10 | 5.0 |
| L230 (pan-αv) | 1.0 |

Examples of antibodies that inhibit LAP adhesion include Exemplary Antibody 5, Exemplary Antibody 17, Exemplary Antibody 11, Exemplary Antibody 14, and Exemplary Antibody 8. Examples of antibodies that do not inhibit LAP adhesion include Exemplary Antibody 18.

### Example 5: α4β1 Vascular Cell Adhesion Protein (VCAM) Adhesion Inhibition

Specificity for the αvβ1 integrin over other RGD binding integrins was established by Octet screening on recombinant protein and/or cell-surface binding to stably transfected cell-lines. Antibodies were also tested for binding to α4β1, a β1-containing integrin that does not bind RGD containing ligands. To confirm the absence of binding to α4β1 in Example 3, antibodies were also tested in a cell adhesion assay to determine if they inhibit the α4β1/ligand (i.e., VCAM) interaction. It is undesirable to exhibit additional cross-reactivity to β1-containing integrins.

A 96-well microtiter plate (Costar 3369) was coated with 100 μl/well of 10 μg/ml VCAM-Ig diluted in 50 mM sodium bicarbonate, pH 9.2, at 4° C. overnight. The plate was washed twice with PBS (200 μl/well), blocked with 1% BSA in PBS (200 μl/well) for 1 h at room temperature, and washed thrice with 200 μl/well of assay buffer (TBS, 2 mM Glucose, 0.1% BSA, 1 mM CaCl₂) and 1 mM MgCl₂, pH 7.4). Jurkat cells were removed from cell suspension and centrifuged at 1500 rpm for 5 mins. The pellet was resuspended in RPMI 1640, 1% BSA buffer and incubated with 2 μM fluorescent dye (BCECF, Molecular Probes, Eugene, Oreg.) in a 37° C. incubator for 30 min. The labeled cells were washed twice by centrifugation at 1100 rpm for 5 min and resuspended in assay buffer to 0.8×10⁶ cells/ml. To individual wells of the washed plate were added 50 μl of purified antibody and 50 μl of Jurkat cells labeled with BCECF, and the plate was incubated at room temperature in dark for 1 h. The plate was washed 3-4 times with assay buffer (200 μl/well), and the fluorescence due to adhered cells on the plate was recorded at 485 nm (Excitation) and 538 nm (Emission) wavelength. Percent binding was determined by comparing the fluorescence prior to the final wash step (i.e. total cells added) to that after washing (i.e. bound cells).

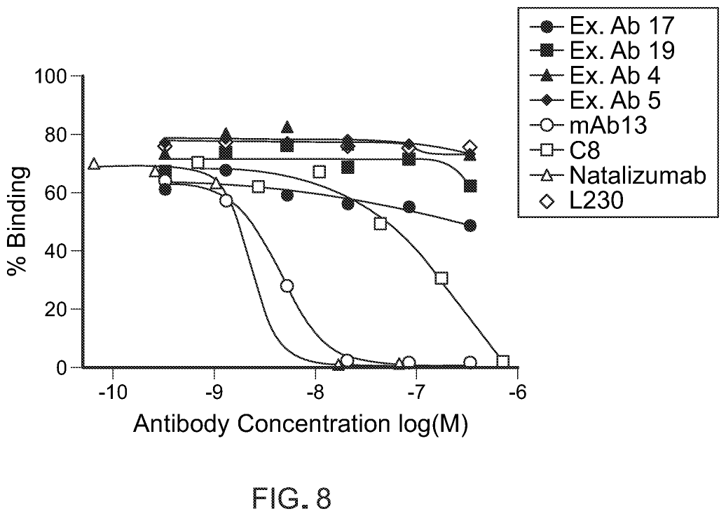

Examples of α4β1 VCAM adhesion inhibition are shown in FIG. 8. This includes examples for a pan-αv commercial antibody (L230), a pan-β1 commercial antibody (mAb13), a published α4-specific antibody (natalizumab), and the c8 small molecule compound previously published as αvβ1 specific (Reed N I et al, Sci Transl Med, 7(288): 288ra79 (2015)). This data confirms that the antibodies do not bind to α4β1 or disrupt cell adhesion. Examples include Exemplary Antibody 17, Exemplary Antibody 19, Exemplary Antibody 4, and Exemplary Antibody 5. The mAb13 (pan-β1) and natalizumab (α4) antibodies inhibit adhesion as expected. The c8 small molecule compound does inhibit α4β1/VCAM adhesion, although α4β1 binding was not specified in the original publication. Subsequent published studies have highlighted potential unknown and undesirable integrin cross-reactivity for the c8 small molecule compound (Wilkinson A L et al, Eur J Pharmacol, 842: 239-247 (2019)). The results from the α4β1 VCAM adhesion inhibition assay confirm c8 binds α4β1 with sufficient affinity to disrupt binding to VCAM.

### Example 6: Fibroblast Binding Assay

Binding to αvβ1 on endogenous cell lines (i.e., non-engineered) is also desirable. In order to directly target fibroblasts, binding of antibodies was tested on MRC9 cells, a human lung fibroblast line, and BLO-11, a mouse skeletal muscle fibroblast line.

MRC9 cells were obtained from Sigma (#85020202) and maintained in EMEM (ATCC, #30-2003) supplemented with 10% Fetal Bovine Serum (FBS from Gibco, #16000-077). BLO-11 cells were obtained from ATCC (#CCL-198) and maintained in DMEM (ATCC, #30-2002) supplemented with 10% FBS. Cells were harvested by incubating with cell dissociation buffer (Gibco, #13150-016) at 37° C. for 10 min and then washed with FACS buffer containing CaCl₂) and MgCl₂ (FACS⁺⁺ buffer; PBS+1% BSA+1 mM CaCl₂)+1 mM MgCl₂). All staining and wash steps were performed at 4° C. in FACS⁺⁺ buffer. In a 96 well U bottom plate, 0.75×10⁶ cells per well were plated and then spun down to remove supernatant. Cells were then resuspended in serially diluted primary antibody and incubated for 30 min. After incubation, cells were washed twice and then incubated with α-human IgG Alexa Fluor 647 (Invitrogen, #A21445) secondary antibody for another 30 min. Cells were then washed twice and fixed with 1% PFA diluted in PBS for 30 min, followed by a final wash. Fluorescence activated cell sorting (FACS) analysis was performed using a five-laser BD LSR-II flow cytometer (BD Biosciences, San Jose, Calif., USA), and data were analyzed using FlowJo software v9 (Treestar, Ashland, Oreg., USA) and transferred into analysis and graphic software including GraphPad Prism 7 (La Jolla, Calif., USA).

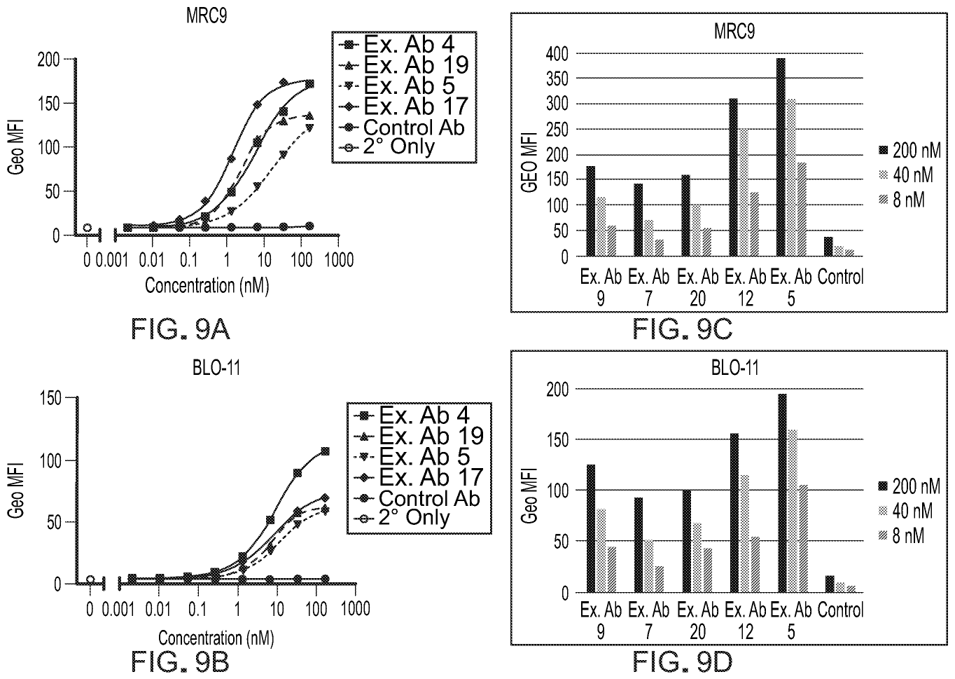

Examples of observed binding to MRC9 (human fibroblast cells) and BLO-11 (murine fibroblast cells) are shown in FIGS. 9A-D. Examples of antibodies that bind the human and mouse fibroblast cell lines include Exemplary Antibody 17, Exemplary Antibody 5, Exemplary Antibody 19, and Exemplary Antibody 4.

### Example 7: LPA-Induced PAI-1 Assay

To determine if blocking αvβ1 with the antibodies will inhibit TGFβ signaling, a cell-based assay was run, using Plasminogen activator inhibitor-1 (PAI-1) gene expression levels as the downstream read-out of TGFβ receptor signaling.

MRC9 cells were obtained from ATCC (#CCL-212) and maintained EMEM (ATCC, #30-2003) supplemented with 10% of heat inactivated Fetal Bovine Serum (FBS #10082, obtained from Gibco). Cells were seeded into laminin-coated 96-well plates (Corning, #354410) at 40,000 cells per well in complete medium (EMEM supplemented with 10% FBS) and incubated overnight at 37° C. with 5% CO₂. Cells were washed the next day with EMEM and serum-starved in EMEM medium for 3 h at 37° C. with 5% CO₂. Cells were then washed twice with EMEM and incubated in EMEM supplemented with 0.1% Bovine Serum Albumin (BSA, obtained from Millipore #126626) in presence or not of inhibitors. After 30 min incubation, cells were then simulated with 5 μM lysophosphatidic acid (LPA, obtained from Sigma-Aldrich #L7260) previously dissolved in EMEM+0.1% BSA. After 20-24 h incubation at 37° C. with 5% CO₂, cell culture medium was removed and plates were stored at −80° C. until qPCR analysis. Gene expression levels of PAI-1 (also known as SERPINE1) and GAPDH were assessed using Taqman Cells-to-CT kit (Ambion, #AM1729), Taqman Gene Expression Master (Ambion #4369016) with Human TaqMan probes (SERPINE 1 #4351368 and GAPDH #4448491 from Applied Biosystems) and qPCR run on Applied Biosystems Viia7 system with analysis completed on Quantstudio Real-Time PCR Software.

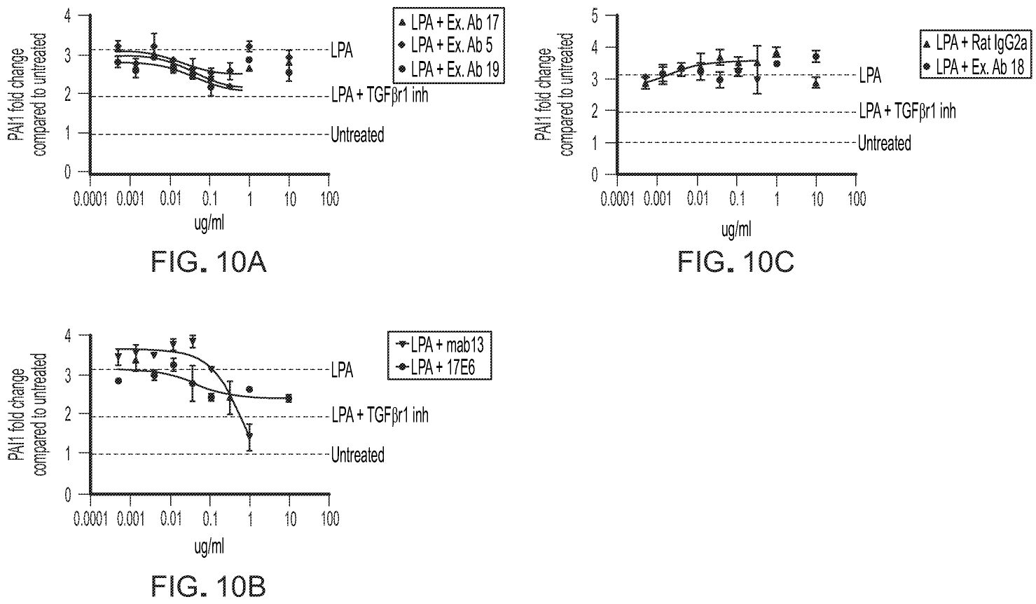

Examples of PAI-1 inhibition are shown in FIGS. 10A-C. This includes examples for a pan-αv commercial antibody (17E6), a pan-β1 commercial antibody (mAb13), and negative control antibodies. Exemplary Antibody 17, Exemplary Antibody 5, and Exemplary Antibody 19 exhibit inhibition of TGFβ signaling.

### Example 8: Methods for Antibody Selections

Recombinant secreted human αvβ1 was purified from the supernatant of transfected CHO cells by co-expression of the individual alpha and beta subunits by methods known in the art (Weinreb P H, J Biol Chem. 2004 Apr. 23; 279(17):17875-87; Chen L L, Cell Commun Adhes. 2008 November; 15(4):317-31; Zhu J, Mol Cell, 2008 Dec. 26; 32 (6), 849-61). Three recombinant versions of integrin αvβ1 were used for selections: (1) the full extracellular regions of αvβ1 with no additional tags, (2) the full extracellular regions of αvβ1 with the β1 fused to the hinge+Fc portion of hIgG1+Avitag (αvβ1-Fc), and (3) the full extracellular regions of αvβ1 with the αv subunit fused to a TEV-acidic coiled coil-StrepII tag and the β1 subunit fused to a TEV-basic coiled coil-6×HIS-G4S-Avitag (αvβ1-cc-AVI) (See, Weinreb et al. J. Biol. Chem. 279(17):17875-17887, 2004; Chen et al. Cell Communication and Adhesions, 15:317-331, 2008; and Zhu et al. Molecular Cell 32:849-861, 2008). In addition, recombinant secreted human αvβ3, αvβ5, αvβ6, αvβ8, α4β1, α5β1, and α8β1 were made as recombinant version #3 (with coiled coil tags) by similar methods. All recombinant integrin proteins were biotinylated for selections.

Selections were performed with the Adimab yeast expression libraries using purified and biotinylated recombinant αvβ1 version 1, 2, and 3. Selections were performed in buffer without cations, as well as buffer containing CaMg, or buffer containing Mn. The first two rounds of selections were performed using Magnetic Activated Cell Sorting (MACS) and all subsequent rounds performed with Fluorescence Activated Cell Sorting (FACS). Utilization of a FACS-based platform allows for visualization of selections of yeast or other cell (prokaryotic or eukaryotic) displayed antibody libraries. With this visualization, selections can be designed to guide to an epitope of interest using guiding proteins (e.g., mAbs, Fabs, ligands, or depletion proteins) (See e.g., Cherf and Cochran, Methods Mol Biol. 1319:155-75, 2015; Xu et al. Protein Eng Des Sel. 26(10):663-70, 2013; and Mann et al. ACS Chem Biol. 8(3):608-16, 2013). If robust enrichment was observed after round 3, subsequent rounds were guided to the epitope of interest using L230 (pan-αv commercial antibody) and/or depletions on αv containing integrins (αvβ3 or αvβ5) and β1 containing integrins (α5β1 or α4β1). Using these conditions, we were able to focus the output to antibodies that bind multiple RGD-binding integrins, as well as antibodies specific for αvβ1. Depending on stringency of depletion, selections led to identification of αvβ1 specific antibodies (e.g. Exemplary antibodies 1, 2, 3) or antibodies that bind multiple RGD-binding integrins (e.g. Exemplary antibodies 15, 16). Subsequent antibody optimization was performed to increase the affinity of certain antibodies for αvβ1 as well as fine-tune the integrin specificity. Antibodies libraries were built using methods known in the art for affinity optimization (e.g. Light Chain shuffling and H-CDR1/H-CDR-2 targeted mutagenesis). After affinity maturation, antibodies were specific for the αvβ1 integrin (e.g. Exemplary antibodies 4 and 5) or bound multiple RGD-binding integrins (e.g. Exemplary antibodies 17, 18, 19).

After αvβ1-specific antibodies were isolated, these antibodies could be used to perform future guiding selections. New selections were performed with the Adimab yeast expression libraries using purified and biotinylated recombinant αvβ1 version 3. Selections were performed in buffer containing CaMg. The first two rounds of selections were performed using MACS and all subsequent rounds performed with FACS. Robust enrichment was observed after round 3, and Exemplary antibody 5 was used to guide to a more refined epitope in round 4 than was achieved in previous selections using L230. Subsequent depletion rounds were performed on αv containing integrins (αvβ3, αvβ5, or αvβ6) and/or β1 containing integrins (α5β1 or α8β1), followed by a positive αvβ1 selection round in some cases. Output from these selections also went through affinity maturation to increase affinity and specificity for αvβ1 or αvβ1l/αvβ6, building libraries using methods known in the art for affinity optimization (i.e. Light Chain shuffling and H-CDR1/H-CDR-2 targeted mutagenesis). After enrichment on αvβ1, depletion rounds were performed with αvβ8 and α5β1 or α8β1, followed by a positive αvβ1 selection round in some cases. Depending on stringency of depletion, affinity optimization selections led to identification of αvβ1 specific antibodies (i.e. Exemplary antibodies 6, 7, 8, 9, 10), αvβ1/αvβ6 specific antibodies (i.e. Exemplary antibodies 11, 12, 13, 14) or antibodies that bind multiple RGD-binding integrins (i.e. Exemplary antibody 20).

Sequences Referenced in this Example:

The amino acid sequence of the human integrin αv protein in recombinant integrin αvβ1 version #1 is shown below.

(SEQ ID NO: 117)
FNLDVDSPAEYSGPEGSYFGFAVDFFVPSASSRMFLLVGAPKANTTQPGI
VEGGQVLKCDWSSTRRCQPIEFDATGNRDYAKDDPLEFKSHQWFGASVRS
KQDKILACAPLYHWRTEMKQEREPVGTCFLQDGTKTVEYAPCRSQDIDAD
GQGFCQGGFSIDFTKADRVLLGGPGSFYWQGQLISDQVAEIVSKYDPNVY
SIKYNNQLATRTAQAIFDDSYLGYSVAVGDFNGDGIDDFVSGVPRAARTL
GMVYIYDGKNMSSLYNFTGEQMAAYFGFSVAATDINGDDYADVFIGAPLF
MDRGSDGKLQEVGQVSVSLQRASGDFQTTKLNGFEVFARFGSAIAPLGDL
DQDGFNDIAIAAPYGGEDKKGIVYIFNGRSTGLNAVPSQILEGQWAARSM
PPSFGYSMKGATDIDKNGYPDLIVGAFGVDRAILYRARPVITVNAGLEVY
PSILNQDNKTCSLPGTALKVSCFNVRFCLKADGKGVLPRKLNFQVELLLD
KLKQKGAIRRALFLYSRSPSHSKNMTISRGGLMQCEELIAYLRDESEFRD
KLTPITIFMEYRLDYRTAADTTGLQPILNQFTPANISRQAHILLDCGEDN
VCKPKLEVSVDSDQKKIYIGDDNPLTLIVKAQNQGEGAYEAELIVSIPLQ
ADFIGVVRNNEALARLSCAFKTENQTRQVVCDLGNPMKAGTQLLAGLRFS
VHQQSEMDTSVKFDLQIQSSNLFDKVSPVVSHKVDLAVLAAVEIRGVSSP
DHVFLPIPNWEHKENPETEEDVGPVVQHIYELRNNGPSSFSKAMLHLQWP
YKYNNNTLLYILHYDIDGPMNCTSDMEINPLRIKISSLQTTEKNDTVAGQ
GERDHLITKRDLALSEGDIHTLGCGVAQCLKIVCQVGRLDRGKSAILYVK
SLLWTETFMNKENQNHSYSLKSSASFNVIEFPYKNLPIEDITNSTLVTTN
VTWGIQPAPMPVP

The amino acid sequence of the human integrin β1 protein in recombinant integrin αvβ1 version #1 is shown below.

(SEQ ID NO: 118)
QTDENRCLKANAKSCGECIQAGPNCGWCTNSTFLQEGMPTSARCDDLEA
LKKKGCPPDDIENPRGSKDIKKNKNVTNRSKGTAEKLKPEDITQIQPQQ
LVLRLRSGEPQTFTLKFKRAEDYPIDLYYLMDLSYSMKDDLENVKSLGT
DLMNEMRRITSDFRIGFGSFVEKTVMPYISTTPAKLRNPCTSEQNCTSP
FSYKNVLSLTNKGEVFNELVGKQRISGNLDSPEGGFDAIMQVAVCGSLI
GWRNVTRLLVFSTDAGFHFAGDGKLGGIVLPNDGQCHLENNMYTMSHYY
DYPSIAHLVQKLSENNIQTIFAVTEEFQPVYKELKNLIPKSAVGTLSAN
SSNVIQLIIDAYNSLSSEVILENGKLSEGVTISYKSYCKNGVNGTGENG
RKCSNISIGDEVQFEISITSNKCPKKDSDSFKIRPLGFTEEVEVILQYI
CECECQSEGIPESPKCHEGNGTFECGACRCNEGRVGRHCECSTDEVNSE
DMDAYCRKENSSEICSNNGECVCGQCVCRKRDNTNEIYSGKFCECDNFN
CDRSNGLICGGNGVCKCRVCECNPNYTGSACDCSLDTSTCEASNGQICN
GRGICECGVCKCTDPKFQGQTCEMCQTCLGVCAEHKECVQCRAFNKGEK
KDTCTQECSYFNITKVESRDKLPQPVQPDPVSHCKEKDVDDCWFYFTYS
VNGNNEVMVHVVENPECPTGPD

The amino acid sequence of the human integrin αv protein in recombinant integrin αvβ1 version #2 is shown below.

(SEQ ID NO: 117)
FNLDVDSPAEYSGPEGSYFGFAVDFFVPSASSRMFLLVGAPKANTTQPG
IVEGGQVLKCDWSSTRRCQPIEFDATGNRDYAKDDPLEFKSHQWFGASV
RSKQDKILACAPLYHWRTEMKQEREPVGTCFLQDGTKTVEYAPCRSQDI
DADGQGFCQGGFSIDFTKADRVLLGGPGSFYWQGQLISDQVAEIVSKYD
PNVYSIKYNNQLATRTAQAIFDDSYLGYSVAVGDFNGDGIDDFVSGVPR
AARTLGMVYIYDGKNMSSLYNFTGEQMAAYFGFSVAATDINGDDYADVF
IGAPLFMDRGSDGKLQEVGQVSVSLQRASGDFQTTKLNGFEVFARFGSA
IAPLGDLDQDGFNDIAIAAPYGGEDKKGIVYIFNGRSTGLNAVPSQILE
GQWAARSMPPSFGYSMKGATDIDKNGYPDLIVGAFGVDRAILYRARPVI
TVNAGLEVYPSILNQDNKTCSLPGTALKVSCFNVRFCLKADGKGVLPRK
LNFQVELLLDKLKQKGAIRRALFLYSRSPSHSKNMTISRGGLMQCEELI
AYLRDESEFRDKLTPITIFMEYRLDYRTAADTTGLQPILNQFTPANISR
QAHILLDCGEDNVCKPKLEVSVDSDQKKIYIGDDNPLTLIVKAQNQGEG
AYEAELIVSIPLQADFIGVVRNNEALARLSCAFKTENQTRQVVCDLGNP
MKAGTQLLAGLRFSVHQQSEMDTSVKFDLQIQSSNLFDKVSPVVSHKVD
LAVLAAVEIRGVSSPDHVFLPIPNWEHKENPETEEDVGPVVQHIYELRN
NGPSSFSKAMLHLQWPYKYNNNTLLYILHYDIDGPMNCTSDMEINPLRI
KISSLQTTEKNDTVAGQGERDHLITKRDLALSEGDIHTLGCGVAQCLKI
VCQVGRLDRGKSAILYVKSLLWTETFMNKENQNHSYSLKSSASFNVIEF
PYKNLPIEDITNSTLVTTNVTWGIQPAPMPVP

The amino acid sequence of the human integrin β1 protein in recombinant integrin αvβ1 version #2 is shown below. The underline denotes the sequence including the hinge, the hIgG1 Fc region and the Avitag.

(SEQ ID NO: 119)
QTDENRCLKANAKSCGECIQAGPNCGWCTNSTFLQEGMPTSARCDDLEA
LKKKGCPPDDIENPRGSKDIKKNKNVTNRSKGTAEKLKPEDITQIQPQQ
LVLRLRSGEPQTFTLKFKRAEDYPIDLYYLMDLSYSMKDDLENVKSLGT
DLMNEMRRITSDFRIGFGSFVEKTVMPYISTTPAKLRNPCTSEQNCTSP
FSYKNVLSLTNKGEVFNELVGKQRISGNLDSPEGGFDAIMQVAVCGSLI
GWRNVTRLLVFSTDAGFHFAGDGKLGGIVLPNDGQCHLENNMYTMSHYY
DYPSIAHLVQKLSENNIQTIFAVTEEFQPVYKELKNLIPKSAVGTLSAN
SSNVIQLIIDAYNSLSSEVILENGKLSEGVTISYKSYCKNGVNGTGENG
RKCSNISIGDEVQFEISITSNKCPKKDSDSFKIRPLGFTEEVEVILQYI
CECECQSEGIPESPKCHEGNGTFECGACRCNEGRVGRHCECSTDEVNSE
DMDAYCRKENSSEICSNNGECVCGQCVCRKRDNTNEIYSGKFCECDNFN
CDRSNGLICGGNGVCKCRVCECNPNYTGSACDCSLDTSTCEASNGQICN
GRGICECGVCKCTDPKFQGQTCEMCQTCLGVCAEHKECVQCRAFNKGEK
KDTCTQECSYFNITKVESRDKLPQPVQPDPVSHCKEKDVDDCWFYFTYS
VNGNNEVMVHVVENPECPTGPDDKTHTCPPCPAPELLGGPSVFLFPPKP
KDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPRE
PQVYTLPPSRDELTKNQVSLTCLVKGFYPSDIAVEWESNGQPENNYKTT
PPVLDSDGSFFLYSKLTVDKSRWQQGNVFSCSVMHEALHNHYTQKSLSL
SPGGGGSGLNDIFEAQKIEWHE

The amino acid sequence of the human integrin αv protein in recombinant integrin αvβ1 version #3 is shown below. The underline denotes the TEV-acidic coiled coil-StrepII tag.

(SEQ ID NO: 120)
FNLDVDSPAEYSGPEGSYFGFAVDFFVPSASSRMFLLVGAPKANTTQPG
IVEGGQVLKCDWSSTRRCQPIEFDATGNRDYAKDDPLEFKSHQWFGASV
RSKQDKILACAPLYHWRTEMKQEREPVGTCFLQDGTKTVEYAPCRSQDI
DADGQGFCQGGFSIDFTKADRVLLGGPGSFYWQGQLISDQVAEIVSKYD
PNVYSIKYNNQLATRTAQAIFDDSYLGYSVAVGDFNGDGIDDFVSGVPR
AARTLGMVYIYDGKNMSSLYNFTGEQMAAYFGFSVAATDINGDDYADVF
IGAPLFMDRGSDGKLQEVGQVSVSLQRASGDFQTTKLNGFEVFARFGSA
IAPLGDLDQDGFNDIAIAAPYGGEDKKGIVYIFNGRSTGLNAVPSQILE
GQWAARSMPPSFGYSMKGATDIDKNGYPDLIVGAFGVDRAILYRARPVI
TVNAGLEVYPSILNQDNKTCSLPGTALKVSCFNVRFCLKADGKGVLPRK
LNFQVELLLDKLKQKGAIRRALFLYSRSPSHSKNMTISRGGLMQCEELI
AYLRDESEFRDKLTPITIFMEYRLDYRTAADTTGLQPILNQFTPANISR
QAHILLDCGEDNVCKPKLEVSVDSDQKKIYIGDDNPLTLIVKAQNQGEG
AYEAELIVSIPLQADFIGVVRNNEALARLSCAFKTENQTRQVVCDLGNP
MKAGTQLLAGLRFSVHQQSEMDTSVKFDLQIQSSNLFDKVSPVVSHKVD
LAVLAAVEIRGVSSPDHVFLPIPNWEHKENPETEEDVGPVVQHIYELRN
NGPSSFSKAMLHLQWPYKYNNNTLLYILHYDIDGPMNCTSDMEINPLRI
KISSLQTTEKNDTVAGQGERDHLITKRDLALSEGDIHTLGCGVAQCLKI
VCQVGRLDRGKSAILYVKSLLWTETFMNKENQNHSYSLKSSASFNVIEF
PYKNLPIEDITNSTLVTTNVTWGIQPAPMPVPTGGLENLYFQGGENAQC
EKELQALEKENAQLEWELQALEKELAQWSHPQFEK

The amino acid sequence of the human integrin β1 protein in recombinant integrin αvβ1 version #3 is shown below. The underline denotes the TEV-basic coiled col-6×HIS-Avitag.

(SEQ ID NO: 121)
QTDENRCLKANAKSCGECIQAGPNCGWCTNSTFLQEGMPTSARCDDLEA
LKKKGCPPDDIENPRGSKDIKKNKNVTNRSKGTAEKLKPEDITQIQPQQ
LVLRLRSGEPQTFTLKFKRAEDYPIDLYYLMDLSYSMKDDLENVKSLGT
DLMNEMRRITSDFRIGFGSFVEKTVMPYISTTPAKLRNPCTSEQNCTSP
FSYKNVLSLTNKGEVFNELVGKQRISGNLDSPEGGFDAIMQVAVCGSLI
GWRNVTRLLVFSTDAGFHFAGDGKLGGIVLPNDGQCHLENNMYTMSHYY
DYPSIAHLVQKLSENNIQTIFAVTEEFQPVYKELKNLIPKSAVGTLSAN
SSNVIQLIIDAYNSLSSEVILENGKLSEGVTISYKSYCKNGVNGTGENG
RKCSNISIGDEVQFEISITSNKCPKKDSDSFKIRPLGFTEEVEVILQYI
CECECQSEGIPESPKCHEGNGTFECGACRCNEGRVGRHCECSTDEVNSE
DMDAYCRKENSSEICSNNGECVCGQCVCRKRDNTNEIYSGKFCECDNFN
CDRSNGLICGGNGVCKCRVCECNPNYTGSACDCSLDTSTCEASNGQICN
GRGICECGVCKCTDPKFQGQTCEMCQTCLGVCAEHKECVQCRAFNKGEK
KDTCTQECSYFNITKVESRDKLPQPVQPDPVSHCKEKDVDDCWFYFTYS
VNGNNEVMVHVVENPECPTGPDTSGLENLYFQGGKNAQCKKKLQALKKK
NAQLKWKLQALKKKLAQGGHHHHHHGGGGSGGGGSGGGGSGGGGSLNDI
FEAQKIEWHE

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An antibody that specifically binds to αvβ1 integrin but not to other integrins, and optionally wherein the antibody has one or more of the following properties: (i) binds with high affinity of KD≤20 nM (bivalent affinity) to human αvβ1; (ii) blocks αvβ1 interaction with its ligand; (iii) is cation-dependent for binding to human αvβ1; (iv) is cation-independent for binding to human αvβ1; (v) binds to αvβ1 on fibroblasts; and (vi) inhibits fibroblast TGFβ response.

2. The antibody of claim 1, wherein the antibody competes with and/or binds the same epitope as a reference anti-αvβ1 integrin antibody comprising a heavy chain variable region (VH) and a light chain variable region (VL), wherein the VH and VL of the reference antibody comprise:
(i) the amino acid sequence set forth in SEQ ID NO:35 and the amino acid sequence set forth in SEQ ID NO:22, respectively;
(ii) the amino acid sequence set forth in SEQ ID NO:61 and the amino acid sequence set forth in SEQ ID NO:58, respectively;
(iii) the amino acid sequence set forth in SEQ ID NO:11 and the amino acid sequence set forth in SEQ ID NO:12, respectively;
(iv) the amino acid sequence set forth in SEQ ID NO:21 and the amino acid sequence set forth in SEQ ID NO:22, respectively;
(v) the amino acid sequence set forth in SEQ ID NO:27 and the amino acid sequence set forth in SEQ ID NO:28, respectively;
(vi) the amino acid sequence set forth in SEQ ID NO:30 and the amino acid sequence set forth in SEQ ID NO:12, respectively;
(vii) the amino acid sequence set forth in SEQ ID NO:44 and the amino acid sequence set forth in SEQ ID NO:45, respectively;
(viii) the amino acid sequence set forth in SEQ ID NO:49 and the amino acid sequence set forth in SEQ ID NO:50, respectively;
(ix) the amino acid sequence set forth in SEQ ID NO:57 and the amino acid sequence set forth in SEQ ID NO:58, respectively; or
(x) the amino acid sequence set forth in SEQ ID NO:64 and the amino acid sequence set forth in SEQ ID NO:58, respectively.

3. An antibody that specifically binds to both αvβ1 and αvβ6 integrins but not to other integrins, and optionally wherein the antibody has one or more of the following properties: (i) bind with high affinity of KD≤20 nM (bivalent affinity) to human αvβ1, and with affinity of 100 nM (bivalent affinity) to human αvβ6; (ii) blocks αvβ1 and/or αvβ6 interaction with its ligand; (iii) is cation-dependent for binding to human αvβ1 and/or αvβ6; (iv) binds to αvβ1 on fibroblasts; and (v) inhibits fibroblast TGFβ response.

4. The antibody of claim 3, wherein the antibody competes with and/or binds the same epitope as a reference antibody that binds both αvβ1 and αvβ6 integrins and comprises a heavy chain variable region (VH) and a light chain variable region (VL), wherein the VH and VL of the reference antibody comprise:
(i) the amino acid sequence set forth in SEQ ID NO: 44 and the amino acid sequence set forth in SEQ ID NO:68, respectively;
(ii) the amino acid sequence set forth in SEQ ID NO:44 and the amino acid sequence set forth in SEQ ID NO:70, respectively;
(iii) the amino acid sequence set forth in SEQ ID NO:49 and the amino acid sequence set forth in SEQ ID NO:72, respectively; or
(iv) the amino acid sequence set forth in SEQ ID NO:76 and the amino acid sequence set forth in SEQ ID NO:77, respectively.

5. An antibody that specifically binds to αvβ1 and one or more integrins selected from the group consisting of αvβ3, αvβ5, αvβ6, αvβ8, α5β1, α8β1, and αIIBβ3, and optionally wherein the antibody has one or more of the following properties: (i) which bind with high affinity of KD≤20 nM (bivalent affinity) to human αvβ1, and with affinity of 100 nM (bivalent affinity) to other RGD binding integrins; (ii) blocks αvβ1 and/or RGD family integrin interaction with its ligand; (iii) is cation-dependent for binding to human αvβ1 and/or RGD binding integrins; (iv) is cation-independent for binding to human αvβ1 and/or RGD binding integrins; (v) binds to αvβ1 and/or RGD binding integrins on fibroblasts; and (vi) inhibits fibroblast TGFβ response.

6. The antibody of claim 5, wherein the antibody competes with and/or binds the same epitope as a reference antibody comprising a heavy chain variable region (VH) and a light chain variable region (VL), wherein the VH and VL of the reference antibody comprise:
(i) the amino acid sequence set forth in SEQ ID NO:82 and the amino acid sequence set forth in SEQ ID NO:83, respectively;
(ii) the amino acid sequence set forth in SEQ ID NO:92 and the amino acid sequence set forth in SEQ ID NO:93, respectively;
(iii) the amino acid sequence set forth in SEQ ID NO:92 and the amino acid sequence set forth in SEQ ID NO:95, respectively;
(iv) the amino acid sequence set forth in SEQ ID NO:100 and the amino acid sequence set forth in SEQ ID NO:28, respectively;
(v) the amino acid sequence set forth in SEQ ID NO:21 and the amino acid sequence set forth in SEQ ID NO:104, respectively; or
(vi) the amino acid sequence set forth in SEQ ID NO:49 and the amino acid sequence set forth in SEQ ID NO:107, respectively.

7. An antibody that binds to αvβ1 integrin but not to other integrins, wherein the antibody comprises a VH comprising VHCDR1, VHCDR2, and VHCDR3, and a VL comprising VLCDR1, VLCDR2, and VLCDR3, wherein VHCDR1, VHCDR2, VHCDR3, VLCDR1, VLCDR2, and VLCDR3 comprise:
(i) SEQ ID NOs:32, 34, 17, 18, 19, and 20, respectively;
(ii) SEQ ID NOs:60, 39, 55, 18, 19, and 56, respectively;
(iii) SEQ ID NOs:4, 6, 7, 8, 9, and 10, respectively;
(iv) SEQ ID NOs:14, 16, 17, 18, 19, and 20, respectively;
(v) SEQ ID NOs:4, 6, 23, 24, 25, and 26, respectively;
(vi) SEQ ID NOs:29, 6, 7, 8, 9, and 10, respectively;
(vii) SEQ ID NOs:37, 39, 40, 41, 42, and 43, respectively;
(viii) SEQ ID NOs:37, 39, 46, 18, 47, and 48, respectively;
(ix) SEQ ID NOs:52, 54, 55, 18, 19, and 56, respectively; or
(x) SEQ ID NOs:63, 54, 55, 18, 19, and 56, respectively.

8. The antibody of claim 7, wherein:
(i) the VH and the VL comprise an amino acid sequence that is at least 75%, 80%, 85%, 90%, 95%, or 100% identical to the amino acid sequences set forth in SEQ ID NOs:35 and 22, respectively;
(ii) the VH and the VL comprise an amino acid sequence that is at least 75%, 80%, 85%, 90%, 95%, or 100% identical to the amino acid sequences set forth in SEQ ID NOs:61 and 58, respectively;
(iii) the VH and the VL comprise an amino acid sequence that is at least 75%, 80%, 85%, 90%, 95%, or 100% identical to the amino acid sequences set forth in SEQ ID NOs:11 and 12, respectively;
(iv) the VH and the VL comprise an amino acid sequence that is at least 75%, 80%, 85%, 90%, 95%, or 100% identical to the amino acid sequences set forth in SEQ ID NOs:21 and 22, respectively;
(v) the VH and the VL comprise an amino acid sequence that is at least 75%, 80%, 85%, 90%, 95%, or 100% identical to the amino acid sequences set forth in SEQ ID NOs:27 and 28, respectively;
(vi) the VH and the VL comprise an amino acid sequence that is at least 75%, 80%, 85%, 90%, 95%, or 100% identical to the amino acid sequences set forth in SEQ ID NOs:30 and 12, respectively;
(vii) the VH and the VL comprise an amino acid sequence that is at least 75%, 80%, 85%, 90%, 95%, or 100% identical to the amino acid sequences set forth in SEQ ID NOs:44 and 45, respectively;
(viii) the VH and the VL comprise an amino acid sequence that is at least 75%, 80%, 85%, 90%, 95%, or 100% identical to the amino acid sequences set forth in SEQ ID NOs:49 and 50, respectively;
(ix) the VH and the VL comprise an amino acid sequence that is at least 75%, 80%, 85%, 90%, 95%, or 100% identical to the amino acid sequences set forth in SEQ ID NOs:57 and 58, respectively; or
(x) the VH and the VL comprise an amino acid sequence that is at least 75%, 80%, 85%, 90%, 95%, or 100% identical to the amino acid sequences set forth in SEQ ID NOs:64 and 58, respectively.

9. An antibody that binds to both αvβ1 and αvβ6 integrins but not to other integrins, wherein the antibody comprises a VH comprising VHCDR1, VHCDR2, and VHCDR3, and a VL comprising VLCDR1, VLCDR2, and VLCDR3, wherein VHCDR1, VHCDR2, VHCDR3, VLCDR1, VLCDR2, and VLCDR3 comprise:
(i) SEQ ID NOs:37, 39, 40, 65, 66, and 67, respectively;
(ii) SEQ ID NOs: 37, 39, 40, 65, 66, and 69, respectively;
(iii) SEQ ID NOs: 37, 39, 46, 18, 47, and 71, respectively; or
(iv) SEQ ID NOs:37, 39, 73, 74, 42, and 75, respectively.

10. The antibody of claim 9, wherein:
(i) the VH and the VL comprise an amino acid sequence that is at least 75%, 80%, 85%, 90%, 95%, or 100% identical to the amino acid sequences set forth in SEQ ID NOs:44 and 68, respectively;
(ii) the VH and the VL comprise an amino acid sequence that is at least 75%, 80%, 85%, 90%, 95%, or 100% identical to the amino acid sequences set forth in SEQ ID NOs:44 and 70, respectively;
(iii) the VH and the VL comprise an amino acid sequence that is at least 75%, 80%, 85%, 90%, 95%, or 100% identical to the amino acid sequences set forth in SEQ ID NOs:49 and 72, respectively; or
(iv) the VH and the VL comprise an amino acid sequence that is at least 75%, 80%, 85%, 90%, 95%, or 100% identical to the amino acid sequences set forth in SEQ ID NOs:76 and 77, respectively.

11. An antibody that binds to αvβ1 and one or more integrins selected from the group consisting of αvβ6, αvβ3, αvβ5, αvβ8, α5β1, α8β1, and αIIBβ3, wherein the antibody comprises a VH comprising VHCDR1, VHCDR2, and VHCDR3, and a VL comprising VLCDR1, VLCDR2, and VLCDR3, wherein VHCDR1, VHCDR2, VHCDR3, VLCDR1, VLCDR2, and VLCDR3 comprise:
(i) SEQ ID NOs:4, 6, 78, 79, 80, and 81, respectively;
(ii) SEQ ID NOs: 85, 87, 88, 89, 90, and 91, respectively;
(iii) SEQ ID NOs: 85, 87, 88, 89, 90, and 94, respectively;
(iv) SEQ ID NOs:97, 99, 23, 24, 25, and 26, respectively;
(v) SEQ ID NOs:14, 16, 17, 101, 102, and 103, respectively; or
(vi) SEQ ID NOs:37, 39, 46, 105, 80, and 106, respectively;

12. The antibody of claim 11, wherein:
(i) the VH and the VL comprise an amino acid sequence that is at least 75%, 80%, 85%, 90%, 95%, or 100% identical to the amino acid sequences set forth in SEQ ID NOs:82 and 83, respectively;
(ii) the VH and the VL comprise an amino acid sequence that is at least 75%, 80%, 85%, 90%, 95%, or 100% identical to the amino acid sequences set forth in SEQ ID NOs:92 and 93, respectively;
(iii) the VH and the VL comprise an amino acid sequence that is at least 75%, 80%, 85%, 90%, 95%, or 100% identical to the amino acid sequences set forth in SEQ ID NOs:92 and 95, respectively;
(iv) the VH and the VL comprise an amino acid sequence that is at least 75%, 80%, 85%, 90%, 95%, or 100% identical to the amino acid sequences set forth in SEQ ID NOs:100 and 28, respectively;
(v) the VH and the VL comprise an amino acid sequence that is at least 75%, 80%, 85%, 90%, 95%, or 100% identical to the amino acid sequences set forth in SEQ ID NOs:21 and 104, respectively; or
(vi) the VH and the VL comprise an amino acid sequence that is at least 75%, 80%, 85%, 90%, 95%, or 100% identical to the amino acid sequences set forth in SEQ ID NOs:49 and 107, respectively.

13. The antibody of any one of claims 1 to 12, wherein the antibody comprises a human IgG1, IgG2, IgG3, or IgG4 heavy chain constant region.

14. The antibody of any one of claims 1 to 12, wherein the antibody comprises an aglycosylated human constant region.

15. The antibody of any one of claims 1 to 12, wherein the antibody comprises an a hIgG1agly Fc, a hIgG2 SAA Fc, a hIgG4(S228P) Fc, or a hIgG4(S228P)/G1 agly Fc.

16. The antibody of any one of claims 1 to 15, wherein the antibody comprises a human kappa or human lambda light chain constant region.

17. The antibody of any one of claims 1 to 12, wherein the antibody is a whole antibody, a single domain antibody, a humanized antibody, a chimeric antibody, a bispecific antibody, a Fv, a scFv, an sc(Fv)2, a diabody, a nanobody, an Fab, and a F(ab′)2.

18. The antibody of any one of claims 1 to 17, further comprising a half-life extending moiety.

19. The antibody of any one of claims 1 to 18, further comprising a detectable label.

20. The antibody of any one of claims 1 to 19, further comprising a therapeutic agent.

21. The antibody of any one of claims 1 to 17, further comprising a radioisotope.

22. The antibody of any one of claims 1 to 17, further comprising a chemotherapeutic or radiotherapeutic agent.

23. A pharmaceutical composition comprising the antibody of any one of claims 1 to 22.

24. A polynucleotide or polynucleotides encoding the antibody of any one of claims 1 to 17.

25. A vector or vectors comprising the polynucleotide or polynucleotides of claim 24.

26. A host cell comprising the polynucleotide or polynucleotides of claim 24, or the vector or vectors of claim 25.

27. A method of making an anti-integrin antibody, the method comprising:
(a) culturing the host cell of claim 26 under conditions that permit expression of the antibody; and
(b) isolating the antibody.

28. The method of claim 27, further comprising formulating the antibody as a sterile formulation suitable for administration to a human.

29. A method of treating or preventing fibrosis in a human subject in need thereof, the method comprising administering to the human subject a therapeutically effective amount of the antibody of any one of claims 1 to 20.

30. The method of claim 29, wherein the fibrosis is selected from the group consisting of liver fibrosis, lung fibrosis, kidney fibrosis, cardiac fibrosis, arthrofibrosis, mediastinal fibrosis, myelofibrosis, nephrogenic systemic fibrosis, Peyronie's disease, progressive massive fibrosis, small airway fibrosis, fibrosis associated with chronic obstructive pulmonary disease, and retroperitoneal fibrosis.

31. The method of claim 30, wherein the fibrosis is liver fibrosis.

32. The method of claim 29, wherein the fibrosis is idiopathic pulmonary fibrosis.

33. The method of claim 29, wherein the fibrosis is scleroderma/systemic sclerosis.

34. A method of treating or preventing chronic kidney disease in a human subject in need thereof, the method comprising administering to the human subject a therapeutically effective amount of the antibody of any one of claims 1 to 20.

35. A method of treating or preventing cancer in a human subject in need thereof, the method comprising administering to the human subject a therapeutically effective amount of the antibody of any one of claims 1 to 22.

36. The method of claim 35, wherein the cancer is of epithelial origin, and optionally wherein the cancer of epithelial origin is a squamous cell carcinoma, an adenocarcinoma, a transitional cell carcinoma, or a basal cell carcinoma.

37. The method of claim 35, wherein the cancer is selected from the group consisting of pancreatic cancer, breast cancer, melanoma, prostate cancer, ovarian cancer, cervical cancer, brain and central nervous system tumors, and glioblastoma.

38. A method of inhibiting platelet aggregation in a human subject in need thereof, the method comprising administering to the human subject a therapeutically effective amount of the antibody of any one of claims 1 to 20.

39. The method of claim 38, wherein the inhibition is for treatment of acute coronary syndrome.

40. A method of treating or preventing an ophthalmology disease or disorder in a human subject in need thereof, the method comprising administering to the human subject a therapeutically effective amount of the antibody of any one of claims 1 to 20.

41. The method of claim 40, wherein the ophthalmology disease or disorder is selected from the group consisting of age-related macular degeneration (AMD), wet AMD, macular edema, and diabetic retinopathy.

42. A method of treating or preventing acute kidney injury, acute lung injury, or acute liver injury in a human subject in need thereof, the method comprising administering to the human subject a therapeutically effective amount of the antibody of any one of claims 1 to 20.

43. A method of treating or preventing Nonalcoholic fatty liver disease (NAFLD) in a human subject in need thereof, the method comprising administering to the human subject a therapeutically effective amount of the antibody of any one of claims 1 to 20.

44. The method of claim 43, wherein the NAFLD is nonalcoholic steatohepatitis (NASH).

45. A method of identifying an antibody that specifically binds to αvβ1 integrin but not to other integrins from a population of antibodies, the method comprising selecting the antibody using guided selection with a guide antibody that is any one of the antibodies of claims 1, 2, 4, or 6-12.

46. The method of claim 45, wherein the population of antibodies comprises an antibody library expressed on the surface of prokaryotic cells.

47. The method of claim 45, wherein the population of antibodies comprises an antibody library expressed on the surface of eukaryotic cells

48. The method of claim 45, wherein the population of antibodies comprises an antibody library expressed on the surface of yeast cells.

49. The method of any one of claims 45 to 48, comprising a step of selecting an antibody that binds to a polypeptide or polypeptides comprising the extracellular domains of αv and β1, optionally wherein the step is performed in the absence of cations, in the presence of calcium and magnesium, or in the presence of manganese, and also optionally, wherein the selection is performed by MACS and/or FACS.

50. The method of any one of claims 45 to 49, further comprising depleting antibodies that bind to one or more integrins selected from the group consisting of αvβ3, αvβ5, αvβ6, αvβ8, α5β1, α8β1, and α4β1.

51. The method of any one of claims 45 to 50, further comprising enriching for antibodies that specifically bind to αvβ1 integrin by selecting for antibodies that bind to αvβ1 integrin.

52. The method of any one of claims 45 to 51, further comprising affinity maturing the selected antibodies.

53. A method of identifying an antibody from a population of antibodies, wherein the antibody specifically binds to both αvβ1 and αvβ6 integrins, the method comprising selecting the antibody using guided selection with a guide antibody that is any one of the antibodies of claims 1, 2, 4, or 6-12.

54. The method of claim 53, wherein the population of antibodies comprises an antibody library expressed on the surface of prokaryotic cells.

55. The method of claim 53, wherein the population of antibodies comprises an antibody library expressed on the surface of eukaryotic cells

56. The method of claim 53, wherein the population of antibodies comprises an antibody library expressed on the surface of yeast cells.

57. The method of any one of claims 53 to 56, comprising a step of selecting an antibody that binds to a polypeptide or polypeptides comprising the extracellular domains of αv and β1 and/or the extracellular domains of αv and β6, optionally wherein the step is performed in the absence of cations, in the presence of calcium and magnesium, or in the presence of manganese, and also optionally, wherein the selection is performed by MACS and/or FACS.

58. The method of any one of claims 53 to 57, further comprising depleting antibodies that bind to one or more integrins selected from the group consisting of αvβ3, αvβ5, αvβ8, α5β1, α8β1, and α4β1.

59. The method of any one of claims 53 to 58, further comprising enriching for antibodies that specifically bind to αvβ1 and αvβ6 integrin by selecting for antibodies that bind to αvβ1 and αvβ6 integrins.

60. The method of any one of claims 53 to 59, further comprising affinity maturing the selected antibodies.
